SEQUENCE LISTING (9 sequences)

SEQ ID NO: 1 - protein, 563 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 2 - DNA, 1693 bp, Homo sapiens
atgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccctgac

SEQ ID NO: 3 - DNA, 1693 bp, Artificial Sequence
  constructed sequence
atgggactgcaggcctgtctgctgggactgttcgccctgatcctgagcggcaagtgcagctacagccccgagcccgaccagagaagaacactgcctccaggctgggtgtccctgggcagagctgaccctgaagaggaactgagcctgaccttcgccctgcggcagcagaacgtggaaagactgagcgagctggtgcaggccgtgtccgatcctagcagccctcagtacggcaagtacctgaccctggaaaacgtggccgacctcgtgcggcctagccctctgacactgcacaccgtgcagaagtggctgctggctgccggcgctcagaaatgccactccgtgatcacccaggactttctgacctgttggctgagcatccggcaggccgaactgctgctgcctggggccgagtttcaccactatgtgggcggacccaccgagacacatgtcgtgcgcagcccacacccttaccagctgccacaggctctggcccctcacgtggactttgtgggaggcctgcacagattccccccaaccagcagcctgagacagaggcctgagccacaagtgaccggcacagtgggcctgcatctgggcgtgacacctagcgtgatccggaagcggtacaacctgaccagccaggatgtgggcagcggcaccagcaacaatagccaggcctgcgcccagttcctggaacagtacttccacgacagcgatctggcccagttcatgcggctgttcggcggcaacttcgcacatcaggctagcgtggccagagtcgtgggccagcagggaagaggcagagccggaattgaggcctccctggacgtgcagtacctgatgagcgctggcgccaacatcagcacctgggtgtacagcagccccggcagacacgagggccaggaaccttttctgcagtggctgatgctgctgagcaacgagagcgccctgcctcatgtgcacacagtgtcctacggcgacgacgaggacagcctgagcagcgcctacatccagagagtgaacaccgagctgatgaaggccgctgccaggggactgaccctgctgtttgcctctggcgatagcggagccggctgttggagtgtgtcaggccggcaccagttcagacccacctttcctgccagctccccctacgtgacaaccgtgggcggcacctcctttcaggaacccttcctgatcaccaacgagatcgtggactacatcagcggcggaggcttcagcaacgtgttccccagacccagctaccaggaagaggccgtgaccaagttcctgtcctccagccctcatctgccccccagctcctacttcaacgccagcggcagagcctacccagatgtggccgctctgtccgacggctactgggtggtgtccaacagagtgcccatcccttgggtgtccggcacaagcgccagcacccctgtgtttggcggcatcctgtccctgatcaacgagcacagaatcctgtccggcagaccccccctgggcttcctgaaccctagactgtaccagcagcacggcgctggcctgttcgatgtgaccagaggctgccacgagagctgcctggacgaggaagtggaaggccagggcttctgttctggccctggctgggatcctgtgaccggatggggcacccctaacttccccgccctgctgaaaacactgctgaacccctgat

SEQ ID NO: 4 - protein, 320 aa, Homo sapiens
MRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 5 - DNA, 6841 bp, Artificial Sequence
  constructed sequence
tcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgccaccatgggactgcaggcctgtctgctgggactgttcgccctgatcctgagcggcaagtgcagctacagccccgagcccgaccagagaagaacactgcctccaggctgggtgtccctgggcagagctgaccctgaagaggaactgagcctgaccttcgccctgcggcagcagaacgtggaaagactgagcgagctggtgcaggccgtgtccgatcctagcagccctcagtacggcaagtacctgaccctggaaaacgtggccgacctcgtgcggcctagccctctgacactgcacaccgtgcagaagtggctgctggctgccggcgctcagaaatgccactccgtgatcacccaggactttctgacctgttggctgagcatccggcaggccgaactgctgctgcctggggccgagtttcaccactatgtgggcggacccaccgagacacatgtcgtgcgcagcccacacccttaccagctgccacaggctctggcccctcacgtggactttgtgggaggcctgcacagattccccccaaccagcagcctgagacagaggcctgagccacaagtgaccggcacagtgggcctgcatctgggcgtgacacctagcgtgatccggaagcggtacaacctgaccagccaggatgtgggcagcggcaccagcaacaatagccaggcctgcgcccagttcctggaacagtacttccacgacagcgatctggcccagttcatgcggctgttcggcggcaacttcgcacatcaggctagcgtggccagagtcgtgggccagcagggaagaggcagagccggaattgaggcctccctggacgtgcagtacctgatgagcgctggcgccaacatcagcacctgggtgtacagcagccccggcagacacgagggccaggaaccttttctgcagtggctgatgctgctgagcaacgagagcgccctgcctcatgtgcacacagtgtcctacggcgacgacgaggacagcctgagcagcgcctacatccagagagtgaacaccgagctgatgaaggccgctgccaggggactgaccctgctgtttgcctctggcgatagcggagccggctgttggagtgtgtcaggccggcaccagttcagacccacctttcctgccagctccccctacgtgacaaccgtgggcggcacctcctttcaggaacccttcctgatcaccaacgagatcgtggactacatcagcggcggaggcttcagcaacgtgttccccagacccagctaccaggaagaggccgtgaccaagttcctgtcctccagccctcatctgccccccagctcctacttcaacgccagcggcagagcctacccagatgtggccgctctgtccgacggctactgggtggtgtccaacagagtgcccatcccttgggtgtccggcacaagcgccagcacccctgtgtttggcggcatcctgtccctgatcaacgagcacagaatcctgtccggcagaccccccctgggcttcctgaaccctagactgtaccagcagcacggcgctggcctgttcgatgtgaccagaggctgccacgagagctgcctggacgaggaagtggaaggccagggcttctgttctggccctggctgggatcctgtgaccggatggggcacccctaacttccccgccctgctgaaaacactgctgaacccctgatgac

SEQ ID NO: 6 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - DNA, 2211 bp, adeno-associated virus 9
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 8 - DNA, 4020 bp, Artificial Sequence
  constructed sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgccaccatgggactgcaggcctgtctgctgggactgttcgccctgatcctgagcggcaagtgcagctacagccccgagcccgaccagagaagaacactgcctccaggctgggtgtccctgggcagagctgaccctgaagaggaactgagcctgaccttcgccctgcggcagcagaacgtggaaagactgagcgagctggtgcaggccgtgtccgatcctagcagccctcagtacggcaagtacctgaccctggaaaacgtggccgacctcgtgcggcctagccctctgacactgcacaccgtgcagaagtggctgctggctgccggcgctcagaaatgccactccgtgatcacccaggactttctgacctgttggctgagcatccggcaggccgaactgctgctgcctggggccgagtttcaccactatgtgggcggacccaccgagacacatgtcgtgcgcagcccacacccttaccagctgccacaggctctggcccctcacgtggactttgtgggaggcctgcacagattccccccaaccagcagcctgagacagaggcctgagccacaagtgaccggcacagtgggcctgcatctgggcgtgacacctagcgtgatccggaagcggtacaacctgaccagccaggatgtgggcagcggcaccagcaacaatagccaggcctgcgcccagttcctggaacagtacttccacgacagcgatctggcccagttcatgcggctgttcggcggcaacttcgcacatcaggctagcgtggccagagtcgtgggccagcagggaagaggcagagccggaattgaggcctccctggacgtgcagtacctgatgagcgctggcgccaacatcagcacctgggtgtacagcagccccggcagacacgagggccaggaaccttttctgcagtggctgatgctgctgagcaacgagagcgccctgcctcatgtgcacacagtgtcctacggcgacgacgaggacagcctgagcagcgcctacatccagagagtgaacaccgagctgatgaaggccgctgccaggggactgaccctgctgtttgcctctggcgatagcggagccggctgttggagtgtgtcaggccggcaccagttcagacccacctttcctgccagctccccctacgtgacaaccgtgggcggcacctcctttcaggaacccttcctgatcaccaacgagatcgtggactacatcagcggcggaggcttcagcaacgtgttccccagacccagctaccaggaagaggccgtgaccaagttcctgtcctccagccctcatctgccccccagctcctacttcaacgccagcggcagagcctacccagatgtggccgctctgtccgacggctactgggtggtgtccaacagagtgcccatcccttgggtgtccggcacaagcgccagcacccctgtgtttggcggcatcctgtccctgatcaacgagcacagaatcctgtccggcagaccccccctgggcttcctgaaccctagactgtaccagcagcacggcgctggcctgttcgatgtgaccagaggctgccacgagagctgcctggacgaggaagtggaaggccagggcttctgttctggccctggctgggatcctgtgaccggatggggcacccctaacttccccgccctgctgaaaacactgctgaacccctgatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 9 - protein, 736 aa, adeno-associated virus 9; identical to SEQ ID NO: 6

(19) United States
(12) Patent Application Publication
(10) Pub. No.: US 2023/0364206 A1
(43) Pub. Date: Nov. 16, 2023

(54) GENE THERAPY FOR OCULAR MANIFESTATIONS OF CLN2 DISEASE

(71) Applicant: REGENXBIO Inc., Rockville, MD (US)

(72) Inventors: Christina Maria Ohnsman, Reading, PA (US); Stephen Joseph Pakola, Irvington, NY (US); Sherri Van Everen, Menlo Park, CA (US); Paulo Falabella, Potomac, MD (US); Alexander Marston Bailey, Washington, DC (US); Nicholas Alexander Piers Sascha Buss, Rockville, MD (US); Kwi Hye Kim, Montgomery Village, MD (US)

(21) Appl. No.: 18/030,613

(22) PCT Filed: Oct. 6, 2021
(86) PCT No.: PCT/US2021/053802

§ 371 Date: Apr. 6, 2023

Related U.S. Application Data
(60) Provisional application No. 63/146,134, filed on Feb. 5, 2021.
(60) Provisional application No. 63/088,911, filed on Oct. 7, 2020.

(51) Int. Cl. A61K 38/48; C12N 9/48; A61K 48/00; C12N 15/86; A61P 27/02

(52) U.S. Cl. CPC A61K 38/4813; C12N 9/485; A61K 48/0066; C12N 15/86; C12Y 304/14009; A61P 27/02; A61K 48/0075; C12N 2750/14143

(57) ABSTRACT

Compositions and methods are described for the delivery of therapeutic products (such as therapeutic proteins (for example, antibodies), therapeutic RNAs (for example, shRNAs, siRNAs, and miRNAs), and therapeutic aptamers) to the retina/vitreal humour in the eyes of human subjects to treat pathologies of the eye, involving, for example, recombinant viral vectors such as recombinant adeno-associated virus (rAAV) vectors.

### PRIORITY

This application claims the benefit of priority to U.S. Ser. No. 63/088,911, filed Oct. 7, 2020, and U.S. Ser. No. 63/146,134, filed Feb. 5, 2021, each of which is incorporated herein by reference in its entirety.

### REFERENCE TO SEQUENCE LISTING SUBMITTED ELECTRONICALLY

This application incorporates by reference a Sequence Listing submitted with this application as text file entitled “12656-144-228_Sequence_Listing.txt” created on Sep. 30, 2021 and having a size of 42,746 bytes.

### 1. BACKGROUND OF THE INVENTION

The neuronal ceroid lipofuscinoses (NCLs) are a group of rare and inherited neurodegenerative disorders. They are considered the most common of the neurogenetic storage diseases, with the accumulation of autofluorescent lipopigments resembling ceroid and lipofuscin seen in patients. NCLs are associated with variable, yet progressive, symptoms, including abnormally increased muscle tone or spasm, blindness or vision problems, dementia, lack of muscle coordination, intellectual disability, movement disorder, seizures and unsteady walk. The frequency of this disease is approximately 1 per 12,500 individuals. There are three main types of NCL: adult (Kufs or Parry disease); juvenile and late infantile (Jansky-Bielschowsky disease). The neuronal ceroid lipofuscinoses (NCLs) originally were defined by their age of onset and clinical symptoms (as noted herein). However, they have since been reclassified on the basis of newer molecular findings, which have provided evidence of far more overlap for the different genetic variants than had previously been suggested by the clinical phenotypes.

At least twenty genes have been identified in association with NCL. NCL patients with CLN2 mutations are deficient in a pepstatin-insensitive lysosomal peptidase called tripeptidyl peptidase 1 (TPP1). TPP1 removes tripeptides from the N-terminal of polypeptides. Mutations have been reported in all 13 exons of the CLN2 gene. Some mutations result in a more protracted course. Although onset is usually in late infancy, later onset has been described. More than 58 mutations have been described in CLN2.

CLN2 disease, a form of Batten disease, is a rare lysosomal storage disorder (LSD) with an estimated incidence of 0.07-0.51 per 100,000 live births (Augestad et al., 2006; Claussen et al., 1992; Mole et al., 2013; National Batten Disease Registry; Poupetova et al., 2010; Santorelli et al., 2013; Teixeira et al., 2003). CLN2 disease is a fatal autosomal recessive neurodegenerative LSD caused by mutations in the CLN2 gene, located on chromosome 11q15 and encoding for the soluble lysosomal enzyme tripeptidyl-peptidase-1 (TPP1). Mutations in the CLN2 gene, and subsequent deficiency in TPP1 enzymatic activity, result in lysosomal accumulation of storage material and neurodegeneration of the brain and retina (Liu et al., 1998; Wlodawer et al., 2003). CLN2 disease is characterized by early onset at 2-4 years of age with initial features usually including recurrent seizures (epilepsy) and difficulty coordinating movements (ataxia). The disease also results in the loss of previously acquired skills (developmental regression). Epilepsy is often refractory to medical therapy, and the general decay of psychomotor functions is rapid and uniform between the third and fifth birthday (Schulz et al., 2013) before premature death by mid-childhood (Nickel M et al., 2016; Worgall et al., 2007).

Retinal degeneration associated with classic neuronal ceroid lipofuscinosis Type 2 (CLN2) disease manifests as a bilateral, progressive, symmetrical decline, beginning at the central macula in a bull's-eye pattern and expanding in late-stage disease to the peripheral retina (Orlin et al, 2013 PLoS One. 2013 Aug. 28; 8 (8):e73128). The retinal degeneration is characterized by rapid loss of central retinal thickness (CRT) on optical coherence tomography (OCT) over a critical 2-year period (Kovacs et al, 2020; Ophthalmol Retina. 2020; 4(7):728-36). However, a less common, attenuated phenotype in which vision loss is delayed may present as late as adolescence (Kohlschûtter et al, 2019).

Enzyme replacement therapy (ERT) with recombinant TPP1 (Brineura® cerliponase alfa, BioMarin Pharmaceuticals) was recently approved in the United States (US) and European Union (EU) for the treatment of CLN2 disease and is administered as a biweekly infusion into the lateral ventricles via a permanently implanted device. The clinical benefit of Brineura® was designated to be limited to stabilization of motor function by the FDA, while the European Medicines Agency (EMA) determined that there was a positive impact on language skills as well (Brineura®, FDA Summary Basis of Approval; Brineura® European Public Assessment Report [EPAR]; Schulz et al., 2016). Brineura® requires specialized expertise for the implantation of a port directly into the brain and must be administered during a 4-hour infusion every two weeks in a healthcare setting by a trained professional knowledgeable in intracerebroventricular (ICV) administration. Repeat infusions are necessary in part due to the short CSF and lysosomal half-lives of Brineura® which are estimated to be 7 hours and 11.5 days, respectively (Brineura®, EPAR). Thus, there remains a significant unmet need for new therapies that can provide durable and long-term TPP1 enzymatic activity in the central nervous system (CNS) of patients with CLN2 disease, without the high patient burden and morbidities associated with repeat administration of ERT. Therefore, compositions useful for delivering and expressing TPP1 in subjects in need for treating CLN2 disease are needed. A one-time administration of recombinant adeno-associated virus (rAAV) expressing canine TPP1 (rAAV2.caTPP1) was shown to result in high expression of TPP1 predominantly in ependymal cells and secretion of the enzyme into the cerebrospinal fluid leading to clinical benefit. See Katz et al, Sci Transl Med. 2015 Nov. 11; 7(313): 313ra180; and KATZ, et al, Gene therapy 2017 Feb. 24(4): 215-223, which are incorporated herein by reference. However AAV2 does not penetrate the brain parenchyma and does not target neurons, thus limiting the expected benefits compared to what can be achieved with novel neurotropic AAVs.

In late infantile neuronal ceroid lipofuscinosis (CLN2) disease, mutations in the CLN2 gene lead to deficient TPP1 enzymatic activity, resulting in profound neurodegeneration within the central nervous system and retina. An extensive number of ocular diseases and diseases with pathological manifestations in the eye can be traced to genetic alterations or protein dysregulations (Stone et al., 2017, Ophthalmology 124(9): 1314-1331). Recent advances in genomics and proteomics have made a huge impact in our understanding of disease mechanisms and/or genetic basis underlying such ocular diseases or manifestations. Gene therapy has been employed in treating certain eye diseases (see, e.g. International Patent Application No. PCT/US2017/027650 (International Publication No. WO 2017/181021 A1)).

There is no currently approved therapy to treat the ocular manifestations of CLN2 disease and visual loss presents a critical unmet need. With the lack of any available treatment options, all children with CLN2 disease will experience macular degeneration associated with vision loss and progression to blindness (Kovacs et al, 2020; Williams et al, 2017; Nickel et al, 2018). Before and since the launch of ERT for treatment of CLN2 disease, leading research institutions have been collecting natural history data to understand the course of retinal degeneration leading to vision loss in the CLN2 population. It has been found that 1) ERT does not alter the trajectory of retinal degeneration associated with CLN2 disease, 2) the onset of retinal degeneration is bilateral, with both eyes affected simultaneously and to the same extent, and 3) retinal degeneration progresses symmetrically between the eyes over time.

### 2. SUMMARY OF THE INVENTION

In one aspect, provided herein is a method of treating ocular manifestations associated with CLN2 Batten disease in a subject in need thereof, said method comprising administering to the eye of said subject a recombinant adeno-associated virus (rAAV) comprising a capsid and a vector genome, wherein the rAAV is AAV9, and wherein the vector genome comprises (a) an AAV 5′ inverted terminal repeat (ITR); (b) a promoter; (c) a CLN2 coding sequence encoding a human tripeptidyl peptidase 1 (TPP1) protein; and (d) an AAV 3′ ITR. In some embodiments, the AAV 5′ ITR and/or AAV3′ ITR is from AAV2. In some embodiments the coding sequence of (c) is a codon optimized human CLN2, which is at least 70% identical to the native human coding sequence of SEQ ID NO: 2. In some embodiments the coding sequence of (c) is SEQ ID NO: 3.

In some embodiments the promoter is a chicken beta actin promoter. In some embodiments the promoter is a hybrid promoter comprising a CBA promoter sequence and cytomegalovirus enhancer elements.

In some embodiments the vector genome further comprises a polyA. In some embodiments the polyA is a synthetic polyA or from bovine growth hormone (bGH), human growth hormone (hGH), SV40, rabbit β-globin (RGB), or modified RGB (mRGB).

In some embodiments, the vector genome further comprises an intron. In some embodiments the intron is from CBA, human beta globin, IVS2, SV40, bGH, alpha-globulin, beta-globulin, collagen, ovalbumin, or p53.

In some embodiments, the vector genome further comprises an enhancer. In some embodiments, the enhancer is a CMV enhancer, an RSV enhancer, an APB enhancer, ABPS enhancer, an alpha mic/bik enhancer, TTR enhancer, en34, ApoE.

In some embodiments, the vector genome is about 3 kilobases to about 5.5 kilobases in size. In some embodiments, the vector genome is about 4 kilobases in size.

In some embodiments, 2×10¹⁰ genome copies per eye of the rAAV are administered. In some embodiments, 6×10¹⁰ genome copies per eye of the rAAV are administered.

In some embodiments, the subject has a change from baseline in central retinal thickness (CRT) as measured by SD-OCT. In some embodiments, the subject has a change from baseline in pupillary light reflex as measured by pupillometry. In some embodiments, the subject has a change from baseline in macular thickness/volume as measured by SD-OCT. In some embodiments, the subject has a change from baseline in outer nuclear layer (ONL) thickness as measured by SD-OCT. In some embodiments, the subject has a change from baseline in full retinal thickness as measured by SD-OCT. In some embodiments, the subject has a change from baseline in the inner nuclear layer as measured by SD-OCT. In some embodiments, the subject has a change from baseline in the photoreceptor (PR) plus in the retinal pigment epithelium (RPE) as measured by SD-OCT. In some embodiments, the subject has a change from baseline in the outer segment plus the RPE (OS+RPE) as measured by SD-OCT. In some embodiments, the subject has a change from baseline in the ellipsoid zone (EZ) as measured by SD-OCT.

In some embodiments, the subject has a delay in the onset of retinal degradation compared to a comparable clinical progression with standard of care. In some embodiments, the subject has a delay in the onset of visual loss compared to a comparable clinical progression with standard of care.

In some embodiments, the method results in detectable TPP1 expression levels in the vitreous humour and/or the aqueous humour of the eye of the subject within 3 months of administration of the rAAV to the subject. In some embodiments, the levels of TPP1 expression in vitreous humour and/or the aqueous humour were undetectable prior to administration of the rAAV. In some embodiments, the leves of TPP1 expression in the serum of the subject remain undetectable.

In some embodiments, the subject is concurrently receiving intracerebroventricular Brineura® enzyme replacement therapy.

In some embodiments, the subject is human.

### 3. BRIEF DESCRIPTION OF DRAWINGS

FIG. 1A is a schematic representation of the AAV.CB7.CI.hTPP1co.RBG vector genome. ITR represents an AAV2 inverted terminal repeat. CB7 represents a chicken beta actin promoter with cytomegalovirus enhancer. RBG PolyA represents a rabbit beta globin polyadenylation signal.

FIG. 1B provides a map of the production plasmid of the AAV.hTPP1co vector.

FIG. 1C provides a map of the AAV cis plasmid construct. ITR: inverted terminal repeat; CMV IE promoter: cytomegalovirus immediate-early promoter; CB promoter: chicken f3-actin promoter Chicken β-actin intron; hCLN2: Human CLN2 cDNA; Rabbit globin poly A: Rabbit beta-globin polyadenylation signal; Kan-r: kanamycin resistance gene.

FIG. 1D provides a map of the AAV trans packaging plasmid construct.

FIG. 1E provides a map of the adenovirus helper plasmid.

FIG. 2 provides a CLN2 CRS-MX scoring flowchart.

FIG. 3 provides a CLN2 CRS-LX scoring flowchart for 2 to <3 Year old subjects.

FIGS. 4A and 4B show TPP1 concentration in both the aqueous and vitreous humor of cynomolgus monkeys after subretinal injection of Construct I. Doses are GC/eye; data presented as Mean +/− SEM.

FIG. 5 shows serum TPP1 concentrations in cynomolgus monkeys after subretinal injection of Construct I. Doses are GC/eye; data presented as Mean +/− SEM.

FIGS. 6A and 6B show vector DNA detected in the eye following subretinal injection of Construct I to cynomolgus monkeys. Data presented as means and standard deviation.

FIGS. 7A and 7B show peripheral biodistribution of Construct I vector DNA after 4 weeks (FIG. 7A) and 13 weeks (FIG. 7B) with subretinal injection. Beneath Limit of Quantification (BLQ): 50 copies/μg DNA. Data presented as means and standard deviation.

FIG. 8 shows Immunofluorescence staining for TPP1 in cynomolgus monkey retina after Construct I administration. Immunofluorescence staining for TPP1; strip of Nissl counterstaining showing the retinal cell layers.

FIG. 9. Thresholding image analysis of TPP1 immunoreactivity. Data shown are compared to vehicle injected animals, a subretinal dose of 1×10¹² GC/eye resulted in significantly greater area of TPP1 immunostaining (** p=0.0013 vs either vehicle, one way ANOVA, post hoc Bonferroni), indicative of a greater level of transduction at this higher dose.

FIG. 10: Transduction of CLN2 Patient RPEs with RGX-381. CLN2 RPEs shows absence of TPP1 expression compared to control RPEs. Transduced CLN2 RPEs in three different doses express TPP1 after 21 days of treatment in dose dependent manner.

FIG. 11: Retinal Organoids Characterisation. Retinal organoids were characterised at day 80 and day 200 of differentiation. TPP1 and Recoverin expressed in day 200, but not in day 80 (FIG. 11A). SCMAS accumulations are detected at day 200 of CLN2 ROs in the absence of TPP1 (FIG. 11B).

FIG. 12: Treatment of ROs with RGX-381. Day 88 ROs were treated with RGX-381 prevented SCMAS accumulation, which was detected in untreated CLN2 ROs. MOI3: Mode of Infection was 3E+06 GC/cells.

### 4. DETAILED DESCRIPTION OF THE INVENTION

Provided herein are methods and compositions for treatment of Batten disease, in particular the treatment of ocular manifestations associated with Batten disease. Ocular manifestations associated with Batten disease include but are not limited to vision loss, retinal atrophy and/or retinal degeneration. Such compositions include a recombinant adeno-associated virus (rAAV), said rAAV comprising an AAV capsid, and a vector genome packaged therein, said vector genome comprising (a) an AAV 5′ inverted terminal repeat (ITR) sequence; (b) a promoter; (c) a CLN2 coding sequence encoding a human TPP1; (d) an AAV 3′ ITR, such as set forth in section 4.1. Also provided herein are methods of manufacturing the rAAV described herein using suspension cell culture, such as described in section 4.1.8. Also provided are compositions comprising recombinant AAV, e.g., compositions described in section 4.2. Also provided herein are methods for treating ocular manifestations of Batten disease caused by a defect in the CLN2 gene comprising delivering to a subject in need thereof a vector (such as rAAV) which encodes TPP1, as described herein, e.g., in section 4.3.

4.1 Constructs and Formulations

For use in the methods provided herein are recombinant viral vectors encoding a therapeutic product (e.g., TPP1). In some embodiments, the vector is a targeted vector, e.g., a vector targeted to retinal pigment epithelial cells.

In some aspects, the disclosure provides for a nucleic acid for use, wherein the nucleic acid encodes a therapeutic product operatively linked to a promoter or enhancer-promoter described herein.

In certain embodiments, provided herein are recombinant vectors that comprise one or more nucleic acids (e.g. polynucleotides). The nucleic acids may comprise DNA, RNA, or a combination of DNA and RNA. In certain embodiments, the DNA comprises one or more of the sequences selected from the group consisting of promoter sequences, the sequence encoding the therapeutic product of interest, untranslated regions, and termination sequences. In certain embodiments, recombinant vectors provided herein comprise a promoter operably linked to the sequence encoding the therapeutic product of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161).

4.1.1 Recombinant Viral Vectors

Recombinant viral vectors include recombinant adenovirus, adeno-associated virus (AAV, e.g., AAV1, AAV2, AAV2tYF, AAV3, AAV4, AAV5, AAV6, AAV7, AAV8, AAV9, AAV10, AAV11, and AAVrh10), lentivirus, helper-dependent adenovirus, herpes simplex virus, poxvirus, hemagglutinin virus of Japan (HVJ), alphavirus, vaccinia virus, and retrovirus vectors. Retroviral vectors include murine leukemia virus (MLV)- and human immunodeficiency virus (HIV)-based vectors. Alphavirus vectors include semliki forest virus (SFV) and sindbis virus (SIN). In certain embodiments, the recombinant viral vectors provided herein are altered such that they are replication-deficient in humans. In certain embodiments, the recombinant viral vectors are hybrid vectors, e.g., an AAV vector placed into a “helpless” adenoviral vector. In certain embodiments, provided herein are recombinant viral vectors comprising a viral capsid from a first virus and viral envelope proteins from a second virus. In specific embodiments, the second virus is vesicular stomatitis virus (VSV). In more specific embodiments, the envelope protein is VSV-G protein.

In certain embodiments, the recombinant viral vectors provided herein are AAV based viral vectors. In preferred embodiments, the recombinant viral vectors provided herein are AAV9-based viral vectors. In certain embodiments, the AAV9-based viral vectors provided herein retain tropism for retinal cells. In certain embodiments, the AAV-based vectors provided herein encode the AAV rep gene (required for replication) and/or the AAV cap gene (required for synthesis of the capsid proteins). Multiple AAV serotypes have been identified. In certain embodiments, AAV-based vectors provided herein comprise components from one or more serotypes of AAV. In certain embodiments, AAV based vectors provided herein comprise capsid components from one or more of AAV1, AAV2, AAV2tYF, AAV3, AAV4, AAV5, AAV6, AAV7, AAV8, AAV9, AAV10, AAV11, or AAVrh10. In preferred embodiments, AAV based vectors provided herein comprise components from one or more of AAV8, AAV9, AAV10, AAV11, or AAVrh10 serotypes.

Provided in particular embodiments are AAV9 vectors comprising a viral genome comprising an expression cassette for expression of the therapeutic product, under the control of regulatory elements and flanked by ITRs and a viral capsid that has the amino acid sequence of the AAV9 capsid protein or is at least 95%, 96%, 97%, 98%, 99% or 99.9% identical to the amino acid sequence of the AAV9 capsid protein (SEQ ID NO: 9) while retaining the biological function of the AAV9 capsid. In certain embodiments, the encoded AAV9 capsid has the sequence of SEQ ID NO: 9 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV9 capsid.

AAV9-based viral vectors are used in certain embodiments of the methods described herein. Nucleic acid sequences of AAV based viral vectors and methods of making recombinant AAV and AAV capsids are taught, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety. In one aspect, provided herein are AAV (e.g., AAV9)-based viral vectors encoding a therapeutic product.

In certain embodiments, a single-stranded AAV (ssAAV) may be used supra. In certain embodiments, a self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

In certain embodiments, the recombinant viral vectors used in the methods described herein is a recombinant adenovirus vector. The recombinant adenovirus can be a first generation vector, with an E1 deletion, with or without an E3 deletion, and with the expression cassette inserted into either deleted region. The recombinant adenovirus can be a second generation vector, which contains full or partial deletions of the E2 and E4 regions. A helper-dependent adenovirus retains only the adenovirus inverted terminal repeats and the packaging signal (phi). The therapeutic product is inserted between the packaging signal and the 3′ITR, with or without stuffer sequences to keep the genome close to wild-type size of approx. 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

4.1.2 Promoters and Modifiers of Gene Expression

In certain embodiments, the recombinant vectors provided herein comprise components that modulate delivery or expression of the therapeutic product (e.g., “expression control elements”). In certain embodiments, the recombinant vectors provided herein comprise components that modulate expression of the therapeutic product. In certain embodiments, the recombinant vectors provided herein comprise components that influence binding or targeting to cells. In certain embodiments, the recombinant vectors provided herein comprise components that influence the localization of the polynucleotide encoding the therapeutic product within the cell after uptake. In certain embodiments, the recombinant vectors provided herein comprise components that can be used as detectable or selectable markers, e.g., to detect or select for cells that have taken up the polynucleotide encoding the therapeutic product.

In certain embodiments, the recombinant vectors provided herein comprise one or more promoters. In certain embodiments, the promoter is a constitutive promoter. In certain embodiments, the promoter is an inducible promoter. Inducible promoters may be preferred so that expression of the therapeutic product may be turned on and off as desired for therapeutic efficacy. Such promoters include, for example, hypoxia-induced promoters and drug inducible promoters, such as promoters induced by rapamycin and related agents. Hypoxia-inducible promoters include promoters with HIF binding sites, see, for example, Schôdel, et al., 2011, Blood 117(23):e207-e217 and Kenneth and Rocha, 2008, Biochem J. 414:19-29, each of which is incorporated by reference for teachings of hypoxia-inducible promoters. In addition, hypoxia-inducible promoters that may be used in the constructs include the erythropoietin promoter and N-WASP promoter (see, Tsuchiya, 1993, J. Biochem. 113:395 for disclosure of the erythropoietin promoter and Salvi, 2017, Biochemistry and Biophysics Reports 9:13-21 for disclosure of N-WASP promoter, both of which are incorporated by reference for the teachings of hypoxia-induced promoters). Alternatively, the recombinant vectors may contain drug inducible promoters, for example promoters inducible by administration of rapamycin and related analogs (see, for example, International Patent Application Publication Nos. WO94/18317, WO 96/20951, WO 96/41865, WO 99/10508, WO 99/10510, WO 99/36553, and WO 99/41258, and U.S. Pat. No. 7,067,526 (disclosing rapamycin analogs), which are incorporated by reference herein for their disclosure of drug inducible promoters). The inducible promoter may also be selected from known promoters including the ecdysone promoter, the estrogen-responsive promoter, and the tetracycline-responsive promoter, or heterodimeric repressor switch. See, Sochor et al, An Autogenously Regulated Expression System for Gene Therapeutic Ocular Applications. Scientific Reports, 2015 Nov. 24; 5:17105 and Daber R, Lewis M., A novel molecular switch. J Mol Biol. 2009 Aug. 28; 391(4):661-70, Epub 2009 Jun. 21 which are both incorporated herein by reference in their entirety.

In certain embodiments the promoter is a hypoxia-inducible promoter. In certain embodiments, the promoter comprises a hypoxia-inducible factor (HIF) binding site. In certain embodiments, the promoter comprises a HIF-1α binding site. In certain embodiments, the promoter comprises a HIF-2α binding site. In certain embodiments, the HIF binding site comprises an RCGTG motif. For details regarding the location and sequence of HIF binding sites, see, e.g., Schôdel, et al., Blood, 2011, 117(23):e207-e217, which is incorporated by reference herein in its entirety. In certain embodiments, the promoter comprises a binding site for a hypoxia induced transcription factor other than a HIF transcription factor. In certain embodiments, the recombinant vectors provided herein comprise one or more IRES sites that is preferentially translated in hypoxia. For teachings regarding hypoxia-inducible gene expression and the factors involved therein, see, e.g., Kenneth and Rocha, Biochem J., 2008, 414:19-29, which is incorporated by reference herein in its entirety.

In another embodiment, the promoter is cell-specific. The term “cell-specific” means that the particular promoter selected for the recombinant vector can direct expression of the optimized TPP1 coding sequence in a particular cell or tissue type.

In another embodiment, the promoter is a ubiquitous or constitutive promoter.

In certain embodiments, the promoter is a CB7 promoter (see Dinculescu et al., 2005, Hum Gene Ther 16: 649-663, incorporated by reference herein in its entirety). In certain embodiments, the CB7 promoter includes other expression control elements that enhance expression of the therapeutic product driven by the vector, e.g. (1) a CAG promoter; (2) a CBA promoter; (3) a CMV promoter; (4) a 1.7-kb red cone opsin promoter (PR1.7 promoter); (5) a Rhodopsin Kinase (GRK1) photoreceptor-specific enhancer-promoter (Young et al., 2003, Retinal Cell Biology; 44:4076-4085); (6) an hCARp promoter, which is a human cone arrestin promoter; (7) an hRKp, which is a rhodopsin kinase promoter; (8) a cone photoreceptor specific human arrestin 3 (ARR3) promoter; (9) a rhodopsin promoter; and (10) a U6 promoter (in particular when the therapeutic product is a small RNA such as shRNA or siRNA).

In certain embodiments, the promoter is a hybrid chicken β-actin (CBA) promoter with cytomegalovirus (CMV) enhancer elements, such as the sequence shown in SEQ ID NO: 5 at nt 3396 to 4061. In another embodiment, the promoter is the CB7 promoter. Other suitable promoters include the human β-actin promoter, the human elongation factor-1a promoter, the cytomegalovirus (CMV) promoter, the simian virus 40 promoter, and the herpes simplex virus thymidine kinase promoter. See, e.g., Damdindorj et al, (August 2014) A Comparative Analysis of Constitutive Promoters Located in Adeno-Associated Viral Vectors. PLoS ONE 9(8): e106472. Still other suitable promoters include viral promoters, constitutive promoters, regulatable promoters (see, e.g., WO 2011/126808 and WO 2013/04943). Alternatively a promoter responsive to physiologic cues may be utilized in the expression cassette, rAAV genomes, vectors, plasmids and viruses described herein. In one embodiment, the promoter is of a small size, under 1000 bp, due to the size limitations of the AAV vector. In another embodiment, the promoter is under 400 bp. Other promoters may be selected by one of skill in the art.

In a further embodiment, the promoter is selected from SV40 promoter, the dihydrofolate reductase promoter, a phage lambda (PL) promoter, a herpes simplex viral (HSV) promoter, a tetracycline-controlled trans-activator-responsive promoter (tet) system, a long terminal repeat (LTR) promoter, such as a RSV LTR, MoMLV LTR, BIV LTR or an HIV LTR, a U3 region promoter of Moloney murine sarcoma virus, a Granzyme A promoter, a regulatory sequence(s) of the metallothionein gene, a CD34 promoter, a CD8 promoter, a thymidine kinase (TK) promoter, a B19 parvovirus promoter, a PGK promoter, a glucocorticoid promoter, a heat shock protein (HSP) promoter, such as HSP65 and HSP70 promoters, an immunoglobulin promoter, an MMTV promoter, a Rous sarcoma virus (RSV) promoter, a lac promoter, a CaMV 35S promoter, a nopaline synthetase promoter, an MND promoter, or an MNC promoter. The promoter sequences thereof are known to one of skill in the art or available publically, such as in the literature or in databases, e.g., GenBank, PubMed, or the like.

In certain embodiments, the other expression control elements include chicken β-actin intron and/or rabbit β-globin polA signal. In certain embodiments, the promoter comprises a TATA box. In certain embodiments, the promoter comprises one or more elements. In certain embodiments, the one or more promoter elements may be inverted or moved relative to one another. In certain embodiments, the elements of the promoter are positioned to function cooperatively. In certain embodiments, the elements of the promoter are positioned to function independently. In certain embodiments, the recombinant vectors provided herein comprise one or more promoters selected from the group consisting of the human CMV immediate early gene promoter, the SV40 early promoter, the Rous sarcoma virus (RS) long terminal repeat, and rat insulin promoter. In certain embodiments, the recombinant vectors provided herein comprise one or more long terminal repeat (LTR) promoters selected from the group consisting of AAV, MLV, MMTV, SV40, RSV, HIV-1, and HIV-2 LTRs. In certain embodiments, the recombinant vectors provided herein comprise one or more tissue specific promoters (e.g., a retinal pigment epithelial cell-specific promoter). In certain embodiments, the recombinant vectors provided herein comprise a RPE65 promoter. In certain embodiments, the recombinant vectors provided herein comprise a VMD2 promoter.

In certain embodiments, the recombinant vectors provided herein comprise one or more regulatory elements other than a promoter. In certain embodiments, the recombinant vectors provided herein comprise an enhancer. In certain embodiments, the recombinant vectors provided herein comprise a repressor. In certain embodiments, the recombinant vectors provided herein comprise an intron or a chimeric intron. In certain embodiments, the recombinant vectors provided herein comprise a polyadenylation sequence.

4.1.3 Signal Peptides

In certain embodiments wherein the therapeutic product is a therapeutic protein, the recombinant vectors provided herein comprise components that modulate protein delivery. In certain embodiments, the recombinant vectors provided herein comprise one or more signal peptides. Signal peptides may also be referred to herein as “leader sequences” or “leader peptides”. In certain embodiments, the signal peptides allow for the therapeutic product to achieve the proper packaging (e.g. glycosylation) in the cell. In certain embodiments, the signal peptides allow for the therapeutic product to achieve the proper localization in the cell. In certain embodiments, the signal peptides allow for the therapeutic product to achieve secretion from the cell. Examples of signal peptides to be used in connection with the recombinant vectors and therapeutic products provided herein may be found in Table 1.

TABLE 1
SIGNAL PEPTIDES FOR USE WITH THE VECTORS PROVIDED HEREIN

| SEQ ID | | |
|---|---|---|
| NO. | Signal Peptide | Sequence |
| 10 | VEGF-A signal peptide | MNFLLSWVHW SLALLLYLHH |
| | | AKWSQA |
| 11 | Fibulin-1 signal peptide | MERAAPSRRV PLPLLLLGGL |
| | | ALLAAGVDA |
| 12 | Vitronectin signal peptide | MAPLRPLLIL ALLAWVALA |
| 13 | Complement Factor H signal | MRLLAKIICLMLWAICVA |
| | peptide | |
| 14 | Opticin signal peptide | MRLLAFLSLL ALVLQETGT |
| 15 | Albumin signal peptide | MKWVTFISLLFLFSSAYS |
| 16 | Chymotrypsinogen signal peptide | MAFLWLLSCWALLGTTFG |
| 17 | Interleukin-2 signal peptide | MYRMQLLSCIALILALVTNS |
| 18 | Trypsinogen-2 signal peptide | MNLLLILTFVAAAVA |
| 19 | mutant Interleukin-2 signal | MYRMQLLLLIALSLALVTNS |
| | peptide | |

4.1.4 Untranslated Regions

In certain embodiments wherein the therapeutic product is a therapeutic protein, the recombinant vectors provided herein comprise one or more untranslated regions (UTRs), e.g., 3′ and/or 5′ UTRs. In certain embodiments, the UTRs are optimized for the desired level of protein expression. In certain embodiments, the UTRs are optimized for the half-life of the mRNA encoding the therapeutic protein. In certain embodiments, the UTRs are optimized for the stability of the mRNA encoding the therapeutic protein. In certain embodiments, the UTRs are optimized for the secondary structure of the mRNA encoding the therapeutic protein.

4.1.5 Inverted Terminal Repeats

In certain embodiments, the recombinant viral vectors provided herein comprise one or more inverted terminal repeat (ITR) sequences. ITR sequences may be used for packaging the recombinant therapeutic product expression cassette into the virion of the recombinant viral vector. In certain embodiments, the ITR is from an AAV, e.g., AAV8 or AAV2 (see, e.g., Yan et al., 2005, J. Virol., 79(1):364-379; U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety).

4.1.6 Therapeutic Product

In another aspect, provided herein is a method of delivering a therapeutic product to a human subject. In some embodiments, the therapeutic product is TPP1. In some embodiments, the patient is experiencing ocular manifestations associated with Batten disease including, without limitation, vision loss, retinal atrophy and/or retinal degeneration. In specific embodiments, provided herein is a method of delivering an effective amount of TPP1 therapeutic product to a subject, wherein the subject experiences ocular manifestations associated with Batten disease.

In certain embodiments of the methods described herein, the therapeutic product is: Tripeptidyl-Peptidase 1 (TPP1). The TPP1 gene, also known as CLN2, encodes Tripeptidyl-peptidase 1, a lysosomal serine protease with tripeptidyl-peptidase I activity. It is also thought to act as a non-specific lysosomal peptidase which generates tripeptides from the breakdown products produced by lysosomal proteinases and requires substrates with an unsubstituted N-terminus. As used herein, the terms “TPP1”, “CLN2”, and “Tripeptidyl-peptidase 1” are used interchangeably when referring to the coding sequence. The native nucleic acid sequence encoding human Tripeptidyl-peptidase 1 is reported at NCBI Reference Sequence NM 000391.3 and reproduced here in SEQ ID NO: 2. Two isoforms of human Tripeptidyl-peptidase 1 has been reported as UniProtKB/Swiss-Prot Accessions 014773-1 and 014773-2 (reproduced here as SEQ ID NOs: 1 and 4). Mutations in the CLN2 gene are associated with late-infantile NCL (LINCL) disease.

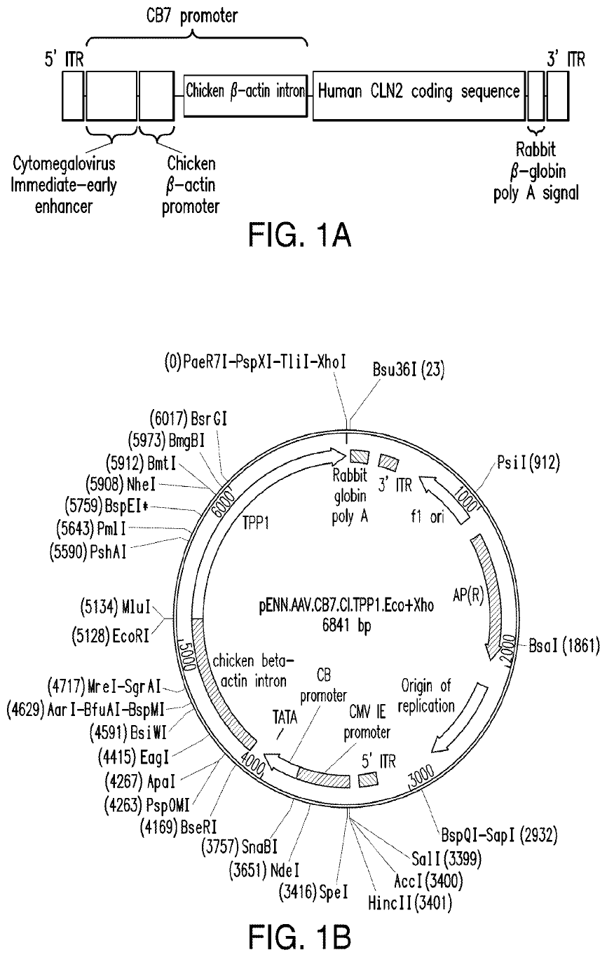

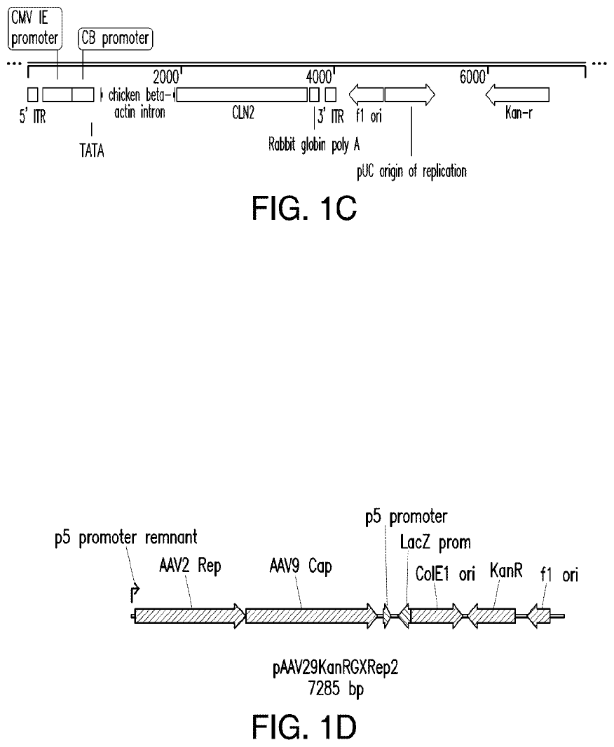

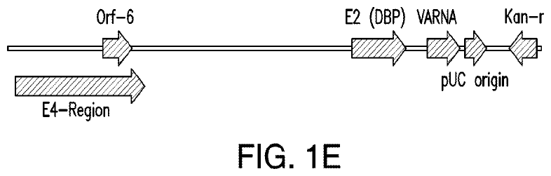

In certain embodiments, the human (h) TPP1-endcoding optimized cDNA may be custom-designed for optimal codon usage and synthesized. In certain embodiments, the hTPP1co cDNA as reproduced as SEQ ID NO: 3 may be then placed in a transgene expression cassette which is driven by a CB7 promoter, a hybrid between a cytomegalovirus (CMV) immediate early enhancer (C4) and the chicken beta actin promoter, while transcription from this promoter is enhanced by the presence of the chicken beta actin intron (CI) (FIGS. 1A and 1B). In certain embodiments, the polyA signal for the expression cassette is the rabbit beta-globin (RBG) polyA.

In one aspect, a codon optimized, engineered nucleic acid sequence encoding human (h) TPP1 is provided. In certain embodiments, an engineered human (h) TPP1 cDNA is provided herein (as SEQ ID NO: 3), which was designed to maximize translation as compared to the native TPP1 sequence (SEQ ID NO: 2). Preferably, the codon optimized TPP1 coding sequence has less than about 80% identity, preferably about 75% identity or less to the full-length native TPP1 coding sequence (SEQ ID NO: 2). In one embodiment, the codon optimized TPP1 coding sequence has about 74% identity with the native TPP1 coding sequence of SEQ ID NO: 2. In one embodiment, the codon optimized TPP1 coding sequence is characterized by improved translation rate as compared to native TPP1 following AAV-mediated delivery (e.g., rAAV). In one embodiment, the codon optimized TPP1 coding sequence shares less than about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61% or less identity to the full length native TPP1 coding sequence of SEQ ID NO: 2. In one embodiment, the codon optimized nucleic acid sequence is a variant of SEQ ID NO: 3. In another embodiment, the codon optimized nucleic acid sequence a sequence sharing about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61% or greater identity with SEQ ID NO: 3. In one embodiment, the codon optimized nucleic acid sequence is SEQ ID NO: 3. In another embodiment, the nucleic acid sequence is codon optimized for expression in humans. In other embodiments, a different TPP1 coding sequence is selected.

The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Identity may be determined by preparing an alignment of the sequences and through the use of a variety of algorithms and/or computer programs known in the art or commercially available [e.g., BLAST, ExPASy; ClustalO; FASTA; using, e.g., Needleman-Wunsch algorithm, Smith-Waterman algorithm]. Alignments are performed using any of a variety of publicly or commercially available Multiple Sequence Alignment Programs. Sequence alignment programs are available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

Multiple sequence alignment programs are also available for nucleic acid sequences. Examples of such programs include, “Clustal Omega”, “Clustal W”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™ a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

Codon-optimized coding regions can be designed by various different methods. This optimization may be performed using methods which are available on-line (e.g., GeneArt), published methods, or a company which provides codon optimizing services, e.g., DNA2.0 (Menlo Park, CA). One codon optimizing method is described, e.g., in US International Patent Publication No. WO 2015/012924, which is incorporated by reference herein in its entirety. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

In one embodiment, the nucleic acid sequence encoding TPP1 further comprises a nucleic acid encoding a tag polypeptide covalently linked thereto. The tag polypeptide may be selected from known “epitope tags” including, without limitation, a myc tag polypeptide, a glutathione-S-transferase tag polypeptide, a green fluorescent protein tag polypeptide, a myc-pyruvate kinase tag polypeptide, a His6 tag polypeptide, an influenza virus hemagglutinin tag polypeptide, a flag tag polypeptide, and a maltose binding protein tag polypeptide.

In another aspect, an expression cassette comprising a nucleic acid sequence that encodes TPP1 is provided. In one embodiment, the sequence is a codon optimized sequence. In another embodiment, the codon optimized nucleic acid sequence is SEQ ID NO: 3 encoding human TPP1.

In some embodiments, the nucleotide sequence encoding TPP1 may be operably linked to a regulatory sequences (including, without limitation, initiator sequences, enhancer sequences, and promoter sequences), which induce, repress, or otherwise control the transcription of protein encoding nucleic acid sequences to which they are operably linked. In some embodiments, the nucleotide sequence encoding TPP1 is a nucleotide sequence described in International Patent Application Publication No. WO 2020/102369, which is incorporated herein by reference herein its entirety.

4.1.7 Constructs

In certain embodiments of the methods described herein, the recombinant vectors provided herein comprise the following elements in the following order: a) a constitutive or a hypoxia-inducible promoter sequence, and b) a sequence encoding the therapeutic product. In certain embodiments, the sequence encoding the therapeutic product comprises multiple ORFs separated by IRES elements. In certain embodiments, the sequence encoding the therapeutic product comprises multiple subunits in one ORF separated by F/F2A sequences.

In certain embodiments of the methods described herein, the recombinant vectors provided herein comprise the following elements in the following order: a) a first ITR sequence, b) a first linker sequence, c) a constitutive or a hypoxia-inducible promoter sequence, d) a second linker sequence, e) an intron sequence, f) a third linker sequence, g) a first UTR sequence, h) a sequence encoding the therapeutic product, i) a second UTR sequence, j) a fourth linker sequence, k) a poly A sequence, 1) a fifth linker sequence, and m) a second ITR sequence.

In certain embodiments, a 6841 bp production plasmid of AAV.hTPP1co vector (AAV.CB7.CI.hTPP1co.RBG) may be constructed with the hTPP1co expression cassette described herein flanked by AAV2 derived ITRs as well as resistance to Ampicillin as a selective marker (FIG. 1B). In certain embodiments, a similar AAV.hTPP1co production plasmid with resistance to Kanamycin may also be constructed. In certain embodiments, the vectors derived from both plasmids may be single-stranded DNA genome with AAV2 derived ITRs flanking the hTPP1co expression cassette described herein.

In certain embodiments, the AAV.hTPP1co vectors may be made by triple transfection and formulated in excipient consisting of phosphate-buffered saline (PBS) containing and 0.001% Pluronic F68 (PF68). See, e.g. Mizukami, Hiroaki, et al., A Protocol for AAV vector production and purification, Diss. Di-vision of Genetic Therapeutics, Center for Molecular Medicine, 1998. In certain embodiments, the genome titers of the vector produced may be determined via droplet digital PCR (ddPCR). See, e.g., M. Lock et al, Hu Gene Therapy Methods, Hum Gene Ther Methods. 2014 April; 25(2):115-25. doi: 10.1089/hgtb.2013.131. Epub 2014 Feb. 14. Described herein is an exemplary AAV.hTPP1co vector, which is sometimes referred to herein as AAV9.CB7.hCLN2.

In some embodiments, the recombinant vector is a recombinant vector described in International Patent Application Publication No. WO 2020/102369, which is incorporated herein by reference herein its entirety.

As used herein, an “expression cassette” refers to a nucleic acid molecule which comprises the coding sequences for TPP1 protein, promoter, and may include other regulatory sequences therefor, which cassette may be packaged into the capsid of a viral vector (e.g., a viral particle). Typically, such an expression cassette for generating a viral vector contains the CLN2 sequences described herein flanked by packaging signals of the viral genome and other expression control sequences such as those described herein. For example, for an AAV viral vector, the packaging signals are the 5′ inverted terminal repeat (ITR) and the 3′ ITR. When packaged into the AAV capsid, the ITRs in conjunction with the expression cassette may be referred to herein as the “recombinant AAV (rAAV) genome” or “vector genome”. In one embodiment, an expression cassette comprises a codon optimized nucleic acid sequence that encodes TPP1 protein. In one embodiment, the cassette provides the codon optimized CLN2 operatively associated with expression control sequences that direct expression of the codon optimized nucleic acid sequence that encodes TPP1 in a host cell.

In another embodiment, an expression cassette for use in an AAV vector is provided. In that embodiment, the AAV expression cassette includes at least one AAV inverted terminal repeat (ITR) sequence. In another embodiment, the expression cassette comprises 5′ ITR sequences and 3′ ITR sequences. In one embodiment, the 5′ and 3′ ITRs flank the codon optimized nucleic acid sequence that encodes TPP1, optionally with additional sequences which direct expression of the codon optimized nucleic acid sequence that encodes TPP1 in a host cell. Thus, as described herein, a AAV expression cassette is meant to describe an expression cassette as described above flanked on its 5′ end by a 5′AAV inverted terminal repeat sequence (ITR) and on its 3′ end by a 3′ AAV ITR. Thus, this rAAV genome contains the minimal sequences required to package the expression cassette into an AAV viral particle, i.e., the AAV 5′ and 3′ ITRs. The AAV ITRs may be obtained from the ITR sequences of any AAV, such as described herein. These ITRs may be of the same AAV origin as the capsid employed in the resulting recombinant AAV, or of a different AAV origin (to produce an AAV pseudotype). In one embodiment, the ITR sequences from AAV2 are used. However, ITRs from other AAV sources may be selected. Where the source of the ITRs is from AAV2 and the AAV capsid is from another AAV source, the resulting vector may be termed pseudotyped. Typically, the AAV vector genome comprises an AAV 5′ ITR, the TPP1 coding sequences and any regulatory sequences, and an AAV 3′ ITR. However, other configurations of these elements may be suitable. A shortened version of the 5′ ITR, termed ΔITR, has been described in which the D-sequence and terminal resolution site (trs) are deleted. In other embodiments, the full-length AAV 5′ and 3′ ITRs are used. Each rAAV genome can be then introduced into a production plasmid.

In one aspect, a vector comprising any of the expression cassettes described herein is provided. As described herein, such vectors can be plasmids of variety of origins and are useful in certain embodiments for the generation of recombinant replication defective viruses as described further herein.

In one embodiment, the vector is a non-viral plasmid that comprises an expression cassette described thereof, e.g., “naked DNA”, “naked plasmid DNA”, RNA, and mRNA; coupled with various compositions and nano particles, including, e.g., micelles, liposomes, cationic lipid—nucleic acid compositions, poly-glycan compositions and other polymers, lipid and/or cholesterol-based—nucleic acid conjugates, and other constructs such as are described herein. See, e.g., X. Su et al, Mol. Pharmaceutics, 2011, 8 (3), pp 774-787; web publication: Mar. 21, 2011; WO2013/182683, WO 2010/053572 and WO 2012/170930, all of which are incorporated herein by reference. Such non-viral TPP1 vector may be administered by the routes described herein. The viral vectors, or non-viral vectors, can be formulated with a physiologically acceptable carrier for use in gene transfer and gene therapy applications.

In another embodiment, the vector is a viral vector that comprises an expression cassette described therein. In some embodiments, the viral vector comprises the exogenous or a heterologous CLN2 nucleic acid transgene. In one embodiment, an expression cassette as described herein may be engineered onto a plasmid which is used for drug delivery or for production of a viral vector. Suitable viral vectors are preferably replication defective and selected from amongst those which target brain cells. Viral vectors may include any virus suitable for gene therapy, including but not limited to adenovirus; herpes virus; lentivirus; retrovirus; parvovirus, etc.

In some embodiments, a viral vector may be replication-deficient. A “replication-defective virus” or “viral vector” refers to a synthetic or recombinant viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

In another embodiment, a recombinant adeno-associated virus (rAAV) vector is provided. The rAAV compromises an AAV capsid, and a vector genome packaged therein.

The vector genome comprises, in one embodiment: (a) an AAV 5′ inverted terminal repeat (ITR) sequence; (b) a promoter; (c) a coding sequence encoding a human TPP1; and (d) an AAV 3′ ITR. In another embodiment, the vector genome is the expression cassette described herein. In one embodiment, the CLN2 sequence encodes a full length TPP1 protein. In one embodiment, the TPP1 sequence is the protein sequence of SEQ ID NO: 1. In another embodiment, the coding sequence is SEQ ID NO: 3 or a variant thereof.

The ITRs or other AAV components may be readily isolated or engineered using techniques available to those of skill in the art from an AAV. Such AAV may be isolated, engineered, or obtained from academic, commercial, or public sources (e.g., the American Type Culture Collection, Manassas, VA). Alternatively, the AAV sequences may be engineered through synthetic or other suitable means by reference to published sequences such as are available in the literature or in databases such as, e.g., GenBank, PubMed, or the like. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

Fragments of AAV may be readily utilized in a variety of vector systems and host cells. Among desirable AAV fragments are the cap proteins, including the vp1, vp2, vp3 and hypervariable regions, the rep proteins, including rep 78, rep 68, rep 52, and rep 40, and the sequences encoding these proteins. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a novel AAV sequence of the invention (e.g., a fragment of a vp1 capsid protein) in combination with heterologous sequences which may be obtained from another AAV serotype (known or novel), non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid. In one embodiment, a vector contains AAV9 cap and/or rep sequences. See, U.S. Pat. No. 7,906,111, which is incorporated by reference herein.

In one embodiment, an AAV vector having AAV9 capsid characterized by the amino acid sequence of SEQ ID NO: 6, is provided herein, in which a nucleic acid encoding a classic late infantile neuronal ceroid lipofuscinosis 2 (CLN2) gene under control of regulatory sequences directing expression thereof in patients in need thereof.

As used herein, an “AAV9 capsid” is characterized by DNAse-resistant particle which is an assembly of about 60 variable proteins (vp) which are typically expressed as alternative splice variants resulting in proteins of different length of SEQ ID NO: 6. See also Genbank Accession No. AAS99264.1, which is incorporated herein by reference. See, also U.S. Pat. No. 7,906,111 and WO 2005/033321. As used herein “AAV9 variants” include those described in, e.g., WO2016/049230, U.S. Pat. No. 8,927,514, US 2015/0344911, and U.S. Pat. No. 8,734,809. The amino acid sequence is reproduced in SEQ ID NO: 6 and the coding sequence is reproduced in SEQ ID NO: 7. In one embodiment, the AAV9 capsid includes a capsid encoded by SEQ ID NO: 7, or a sequence sharing at least about 90%, 95%, 95%, 98% or 99% identity therewith.

The largest protein, vp1, is generally the full-length of the amino acid sequence of SEQ ID NO: 6 (aa 1-736 of SEQ ID NO: 6). In certain embodiments, the AAV9 vp2 protein has the amino acid sequence of 138 to 736 of SEQ ID NO: 6. In certain embodiments, the AAV9 vp3 has the amino acid sequence of 203 to 736 of SEQ ID NO: 6. In certain embodiments, the vp 1, 2 or 3 proteins may be have truncations (e.g., 1 or more amino acids at the N-terminus or C-terminus). An AAV9 capsid is composed of about 60 vp proteins, in which vp1, vp2 and vp3 are present in a ratio of about 1 vp, to about 1 vp2, to about 10 to 20 vp3 proteins within the assembled capsid. This ratio may vary depending upon the production system used. In certain embodiments, an engineered AAV9 capsid may be generated in which vp2 is absent.

It is within the skill in the art to design nucleic acid sequences encoding this AAV9 capsid, including DNA (genomic or cDNA), or RNA (e.g., mRNA). In certain embodiments, the nucleic acid sequence encoding the AAV9 vp1 capsid protein is provided in SEQ ID NO: 7. In other embodiments, a nucleic acid sequence of 70% to 99.9% identity to SEQ ID NO: 7 may be selected to express the AAV9 capsid. In certain other embodiments, the nucleic acid sequence is at least about 75% identical, at least 80% identical, at least 85%, at least 90%, at least 95%, at least 97% identical, or at least 99% to 99.9% identical to SEQ ID NO: 7.

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics, Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another Glade, or is outside these clades. See, e.g., G Gao, et al, J Virol, 2004 June; 78(10): 6381-6388, which identifies Clades A, B, C, D, E and F, and provides nucleic acid sequences of novel AAV, GenBank Accession Numbers AY530553 to AY530629. See, also, WO 2005/033321. AAV9 is further characterized by being within Clade F. Other Clade F AAV include AAVhu31 and AAVhu32.

As used herein, relating to AAV, the term variant means any AAV sequence which is derived from a known AAV sequence, including those sharing at least 70%, at least 75%, at least 80%, at least 85%, at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV9 capsid. When determining the percent identity of an AAV capsid, the comparison may be made over any of the variable proteins (e.g., vp1, vp2, or vp3). In one embodiment, the AAV capsid shares at least 95% identity with the AAV9 over the vp1, vp2 or vp3.

As used herein, “artificial AAV” means, without limitation, an AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the invention. In one embodiment, AAV2/9 and AAV2/rh.10 are exemplary pseudotyped vectors.

In another embodiment, a self-complementary AAV is used. “Self-complementary AAV” refers a plasmid or vector having an expression cassette in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

In still another embodiment, the expression cassette, including any of those described herein is employed to generate a recombinant AAV genome.

In one embodiment, the expression cassette described herein is engineered into a suitable genetic element (vector) useful for generating viral vectors and/or for delivery to a host cell, e.g., naked DNA, phage, transposon, cosmid, episome, etc., which transfers the CLN2 sequences carried thereon. The selected vector may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, NY.

For packaging an expression cassette or rAAV genome or production plasmid into virions, the ITRs are the only AAV components required in cis in the same construct as the expression cassette. In one embodiment, the coding sequences for the replication (rep) and/or capsid (cap) are removed from the AAV genome and supplied in trans or by a packaging cell line in order to generate the AAV vector.

Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2. In a one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In a specific embodiment, the producer cell line or packaging cell line is a suspension cell line such that the AAV viral vectors described herein can be manufactured by growing the producer cell line or packaging cell line in suspension culture. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level.

In yet another system, the expression cassette flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of which is incorporated herein by reference in its entirety. Methods of making and using these and other AAV production systems are also described in the following U.S. patents, the contents of each of which is incorporated herein by reference in its entirety: 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety.

In one aspect, provided herein is a method of manufacturing an rAAV described herein, comprising growing in suspension culture a suspension cell line that is capable of producing the rAAV.

In certain embodiments, the suspension cell line is derived from an adherent cell line by adaptation of cells into suspension culture using serum-free and animal component-free culture medium. In a specific embodiment, the suspension cell line is HEK293 suspension cell line.

The methods used to construct any embodiment of this invention are known to those with skill in nucleic acid manipulation and include genetic engineering, recombinant engineering, and synthetic techniques. See, e.g., Green and Sambrook et al, Molecular Cloning: A Laboratory Manual, Cold Spring Harbor Press, Cold Spring Harbor, NY (2012). Similarly, methods of generating rAAV virions are well known and the selection of a suitable method is not a limitation on the present invention. See, e.g., K. Fisher et al, (1993) J. Virol., 70:520-532 and U.S. Pat. No. 5,478,745.

“Plasmids” generally are designated herein by a lower case p preceded and/or followed by capital letters and/or numbers, in accordance with standard naming conventions that are familiar to those of skill in the art. Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

In one embodiment, the production plasmid is that described herein, or as described in WO2012/158757, which is incorporated herein by reference. Various plasmids are known in the art for use in producing rAAV vectors, and are useful herein. The production plasmids are cultured in the host cells which express the AAV cap and/or rep proteins. In the host cells, each rAAV genome is rescued and packaged into the capsid protein or envelope protein to form an infectious viral particle.

In one aspect, a production plasmid comprising an expression cassette described above is provided. In one embodiment, the production plasmid is that shown in FIG. 1B. This plasmid is used in the examples for generation of the rAAV-human codon optimized TPP1 vector. Such a plasmid is one that contains a 5′ AAV ITR sequence; a selected promoter; a polyA sequence; and a 3′ ITR; additionally, it also contains an intron sequence, such as the chicken beta-actin intron. An exemplary schematic is shown in FIG. 1A. In a further embodiment, the intron sequence keeps the rAAV vector genome with a size between about 3 kilobases (kb) to about 6 kb, about 4.7 kb to about 6 kb, about 3 kb to about 5.5 kb, or about 4.7 kb to 5.5 kb. An example of a production plasmid which includes the TPP1 encoding sequence can be found in SEQ ID NO: 5. In another embodiment, the production plasmid is modified to optimized vector plasmid production efficiency. Such modifications include addition of other neutral sequences, or inclusion of a lambda stuffer sequence to modulate the level of supercoil of the vector plasmid. Such modifications are contemplated herein. In other embodiments, terminator and other sequences are included in the plasmid.

In certain embodiments, the rAAV expression cassette, the vector (such as rAAV vector), the virus (such as rAAV), and/or the production plasmid comprises AAV inverted terminal repeat sequences, a codon optimized nucleic acid sequence that encodes TPP1, and expression control sequences that direct expression of the encoded proteins in a host cell. In other embodiments, the rAAV expression cassette, the virus, the vector (such as rAAV vector), and/or the production plasmid further comprise one or more of an intron, a Kozak sequence, a polyA, post-transcriptional regulatory elements and others. In one embodiment, the post-transcriptional regulatory element is Woodchuck Hepatitis Virus (WHP) Posttranscriptional Regulatory Element (WPRE).

The expression cassettes, vectors and plasmids include other components that can be optimized for a specific species using techniques known in the art including, e.g., codon optimization, as described herein.

In other embodiments, the expression cassette, vector, plasmid and virus described herein contain other appropriate transcription initiation, termination, enhancer sequences, efficient RNA processing signals such as splicing and polyadenylation (polyA) signals; TATA sequences; sequences that stabilize cytoplasmic mRNA; sequences that enhance translation efficiency (i.e., Kozak consensus sequence); introns; sequences that enhance protein stability; and when desired, sequences that enhance secretion of the encoded product. The expression cassette or vector may contain none, one or more of any of the elements described herein.

Examples of suitable polyA sequences include, e.g., a synthetic polyA or from bovine growth hormone (bGH), human growth hormone (hGH), SV40, rabbit β-globin (RGB), or modified RGB (mRGB). In a further embodiment, the poly A has a nucleic acid sequence from nt 33 to 159 of SEQ ID NO: 5.

Examples of suitable enhancers include, e.g., the CMV enhancer, the RSV enhancer, the alpha fetoprotein enhancer, the TTR minimal promoter/enhancer, LSP (TH-binding globulin promoter/alpha1-microglobulin/bikunin enhancer), an APB enhancer, ABPS enhancer, an alpha mic/bik enhancer, TTR enhancer, en34, ApoE amongst others.

In one embodiment, a Kozak sequence is included upstream of the TPP1 coding sequence to enhance translation from the correct initiation codon. In another embodiment, CBA exon 1 and intron are included in the expression cassette. In one embodiment, the TPP1 coding sequence is placed under the control of a hybrid chicken β actin (CBA) promoter. This promoter consists of the cytomegalovirus (CMV) immediate early enhancer, the proximal chicken β actin promoter, and CBA exon 1 flanked by intron 1 sequences.

In another embodiment, the intron is selected from CBA, human beta globin, IVS2, SV40, bGH, alpha-globulin, beta-globulin, collagen, ovalbumin, p53, or a fragment thereof.

In one embodiment, the expression cassette, the vector, the plasmid and the virus contain a 5′ ITR, chicken beta-actin (CBA) promoter, CMV enhancer, CBA exon 1 and intron, human codon optimized CLN2 sequence, rabbit globin poly A and 3′ ITR. In a further embodiment, the expression cassette includes nt 1 to 4020 of SEQ ID NO: 8. In yet a further embodiment, the 5′ ITR has a nucleic acid sequence from nt 3199 to nt 3328 of SEQ ID NO: 5 and the 3′ITR has a nucleic acid sequence from nt 248 to nt 377 of SEQ ID NO: 5. In a further embodiment, the production plasmid has a sequence of SEQ ID NO: 5, also shown in FIGS. 1C-1E.

4.1.8 Manufacture and Testing of Vectors

The recombinant vectors (for example, recombinant viral vectors) provided herein may be manufactured using host cells. The recombinant vectors provided herein may be manufactured using mammalian host cells, for example, A549, WEHI, 10T1/2, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. The recombinant vectors provided herein may be manufactured using host cells from human, monkey, mouse, rat, rabbit, or hamster. In other embodiments, the term “host cell” is intended to reference the brain cells of the subject being treated in vivo for Batten disease. Such host cells include epithelial cells of the CNS including ependyma, the epithelial lining of the brain ventricular system. Other host cells include neurons, astrocytes, oligoedendrocytes, and microglia.

For recombinant viral vectors, the host cells are stably transformed with the sequences encoding the therapeutic product and associated elements (i.e., the vector genome), and the means of producing viruses in the host cells, for example, the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation.

In vitro assays, e.g., cell culture assays, can be used to measure therapeutic product expression from a vector described herein, thus indicating, e.g., potency of the vector. For example, the PER.C6® Cell Line (Lonza), a cell line derived from human embryonic retinal cells, or retinal pigment epithelial cells, e.g., the retinal pigment epithelial cell line hTERT RPE-1 (available from ATCC®), can be used to assess therapeutic product expression. Once expressed, characteristics of the expressed therapeutic product can be determined, including determination of the post-translational modification patterns. In addition, benefits resulting from post-translational modification of the cell-expressed therapeutic product can be determined using assays known in the art.

4.2 Compositions

Compositions are described comprising a recombinant vector encoding a therapeutic product described herein and a suitable carrier. A suitable carrier (e.g., for suprachoroidal, subretinal, juxtascleral, intravitreal, subconjunctival, and/or intraretinal administration) would be readily selected by one of skill in the art.

The disclosure provides a pharmaceutical composition comprising a recombinant adeno-associated virus (AAV), potassium phosphate monobasic, sodium chloride, sodium phosphate dibasic anhydrous, sucrose, and surfactant.

In some embodiments, the disclosure provides a pharmaceutical composition comprising a recombinant adeno-associated virus (AAV), ionic salt excipient or buffering agent, sucrose, and poloxamer 188. In some embodiments, the ionic salt excipient or buffering agent can be one or more components from the group consisting of potassium phosphate monobasic, potassium phosphate, sodium chloride, sodium phosphate dibasic anhydrous, sodium phosphate hexahydrate, sodium phosphate monobasic monohydrate, tromethamine, tris(hydroxymethyl)aminomethane hydrochloride (Tris-HCl), amino acid, histidine, histidine hydrochloride (histidine-HCl), sodium succinate, sodium citrate, sodium acetate, and (4-(2-hydroxyethyl)-1-piperazineethanesulfonic acid) (HEPES), sodium sulfate, magnesium sulfate, magnesium chloride 6-hydrate, calcium sulfate, potassium chloride, calcium chloride, calcium citrate.

In certain embodiments, the pharmaceutical composition has a ionic strength about 60 mM to 115 mM. In certain embodiments, the pharmaceutical composition has a ionic strength about 60 mM to 100 mM. In certain embodiments, the pharmaceutical composition has a ionic strength about 65 mM to 95 mM. In certain embodiments, the pharmaceutical composition has a ionic strength about 70 mM to 90 mM. In certain embodiments, the pharmaceutical composition has a ionic strength about 75 mM to 85 mM.

In certain embodiments, the pharmaceutical composition has a ionic strength about 30 mM to 100 mM. In certain embodiments, the pharmaceutical composition has a ionic strength about 35 mM to 95 mM. In certain embodiments, the pharmaceutical composition has a ionic strength about 40 mM to 90 mM. In certain embodiments, the pharmaceutical composition has a ionic strength about 45 mM to 85 mM. In certain embodiments, the pharmaceutical composition has a ionic strength about 50 mM to 80 mM. In certain embodiments, the pharmaceutical composition has a ionic strength about 55 mM to 75 mM. In certain embodiments, the pharmaceutical composition has a ionic strength about 60 mM to 70 mM.

In certain embodiments, the pharmaceutical composition has a ionic strength ranging from 60 mM to 115 mM. In certain embodiments, the pharmaceutical composition has a ionic strength ranging from 60 mM to 100 mM. In certain embodiments, the pharmaceutical composition has a ionic strength ranging from 65 mM to 95 mM. In certain embodiments, the pharmaceutical composition has a ionic strength ranging from 70 mM to 90 mM. In certain embodiments, the pharmaceutical composition has a ionic strength ranging from 75 mM to 85 mM.

In certain embodiments, the pharmaceutical composition has a ionic strength range from 30 mM to 100 mM. In certain embodiments, the pharmaceutical composition has a ionic strength range from 35 mM to 95 mM. In certain embodiments, the pharmaceutical composition has a ionic strength range from 40 mM to 90 mM. In certain embodiments, the pharmaceutical composition has a ionic strength range from 45 mM to 85 mM. In certain embodiments, the pharmaceutical composition has a ionic strength range from 50 mM to 80 mM. In certain embodiments, the pharmaceutical composition has a ionic strength range from 55 mM to 75 mM. In certain embodiments, the pharmaceutical composition has a ionic strength range from 60 mM to 70 mM.

In certain embodiments, the pharmaceutical composition comprises potassium chloride at a concentration of 0.2 g/L.

In certain embodiments, the pharmaceutical composition comprises potassium phosphate monobasic at a concentration of 0.2 g/L.

In certain embodiments, the pharmaceutical composition comprises sodium chloride at a concentration of 5.84 g/L, and

In certain embodiments, the pharmaceutical composition comprises sodium phosphate dibasic anhydrous at a concentration of 1.15 g/L.

In certain embodiments, the pharmaceutical composition comprises sucrose at a concentration of 3% (weight/volume, 30 g/L) to 18% (weight/volume, 180 g/L). In certain embodiments, the pharmaceutical composition comprises sucrose at a concentration of 4% (weight/volume, 40 g/L).

In certain embodiments, the pharmaceutical composition comprises poloxamer 188 at a concentration of 0.001% (weight/volume, 0.01 g/L).

In certain embodiments, the pharmaceutical composition comprises poloxamer 188 at a concentration of 0.0005% (weight/volume, 0.005 g/L) to 0.05% (weight/volume, 0.5 g/L). In certain embodiments, the pharmaceutical composition comprises poloxamer 188 at a concentration of 0.001% (weight/volume, 0.01 g/L).

In some embodiments, the disclosure provides a pharmaceutical composition comprises a recombinant adeno-associated virus (AAV), ionic salt excipient or buffering agent, sucrose, and surfactant. In some embodiments, the ionic salt excipient or buffering agent can be one or more components from the group consisting of potassium phosphate monobasic, potassium phosphate, sodium chloride, sodium phosphate dibasic anhydrous, sodium phosphate hexahydrate, sodium phosphate monobasic monohydrate, tromethamine, tris(hydroxymethyl)aminomethane hydrochloride (Tris-HCl), amino acid, histidine, histidine hydrochloride (histidine-HCl), sodium succinate, sodium citrate, sodium acetate, and (4-(2-hydroxyethyl)-1-piperazineethanesulfonic acid) (HEPES), sodium sulfate, magnesium sulfate, magnesium chloride 6-hydrate, calcium sulfate, potassium chloride, calcium chloride, calcium citrate. In some embodiments, the surfactant can be one or more components from the group consisting of poloxamer 188, polysorbate 20, and polysorbate 80.

In certain embodiments, the pharmaceutical composition comprises polysorbate 20 at a concentration of 0.0005% (weight/volume, 0.05 g/L) to 0.05% (weight/volume, 0.5 g/L).

In certain embodiments, the pharmaceutical composition comprises polysorbate 80 at a concentration of 0.0005% (weight/volume, 0.05 g/L) to 0.05% (weight/volume, 0.5 g/L).

In certain embodiments, the pH of the pharmaceutical composition is about 7.4.

In certain embodiments, the pH of the pharmaceutical composition is about 6.0 to 9.0.

In certain embodiments, the pH of the pharmaceutical composition is 7.4.

In certain embodiments, the pH of the pharmaceutical composition is 6.0 to 9.0.

As used herein and unless otherwise specified, the term “about” means within plus or minus 10% of a given value or range.

In certain embodiments, the pharmaceutical composition is in a hydrophobically-coated glass vial.

In certain embodiments, the pharmaceutical composition is in a Cyclo Olefin Polymer (COP) vial.

In certain embodiments, the pharmaceutical composition is in a Daikyo Crystal Zenith® (CZ) vial.

In certain embodiments, the pharmaceutical composition is in a TopLyo coated vial.

In certain embodiments, disclosed herein is a pharmaceutical composition consists of: (a) the recombinant AAV, (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water, and wherein the recombinant AAV is AAV9.

In certain embodiments, the vector genome concentration (VGC) of the pharmaceutical composition is about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 1×10¹¹ GC/mL, about 2×10¹¹ GC/mL, about 2.4×10¹¹ GC/mL, about 2.5×10¹¹ GC/mL, about 3×10¹¹ GC/mL, about 6.2×10¹¹ GC/mL, about 1×10¹² GC/mL, about 3×10¹² GC/mL, about 2×10¹³ GC/mL or about 3×10¹³ GC/mL

In certain embodiments, the disclosure provides a pharmaceutical composition or formulation comprising a recombinant adeno-associated virus (AAV), potassium phosphate monobasic, sodium chloride, sodium phosphate dibasic anhydrous, sucrose, and poloxamer 188. In some embodiments, the recombinant AAV comprises components from AAV9. In some embodiments, the AAV comprises an AAV capsid, and a vector genome packaged therein, said vector genome comprising: (a) an AAV 5′ inverted terminal repeat (ITR) sequence; (b) a promoter; (c) a CLN2 coding sequence encoding a human TPP1; and (d) an AAV 3′ ITR.

In a preferred embodiment, the AAV used for delivering the transgene should have a tropism for human retinal cells or photoreceptor cells. Such AAV can include non-replicating recombinant adeno-associated virus vectors, particularly those bearing an AAV9 capsid are preferred. In another specific embodiment, the viral vector or other DNA expression construct described herein is Construct I, wherein the Construct I comprise the following components: an AAV capsid, and a vector genome packaged therein, said vector genome comprising: (a) an AAV 5′ inverted terminal repeat (ITR) sequence; (b) a promoter; (c) a CLN2 coding sequence encoding a human TPP1; and (d) an AAV 3′ ITR.

In some embodiments, the pharmaceutical composition consists of: (a) an AAV capsid packaging vector encoding a transgene of interest, (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water, and wherein the transgene of interest encodes an RNA of interest or a protein of interest, for example human TPP1.

In some embodiments, the pharmaceutical composition is a liquid composition. In some embodiments, the pharmaceutical composition is a frozen composition. In some embodiments, the pharmaceutical composition is a lyophilized composition from a liquid composition disclosed herein. In some embodiments, the pharmaceutical composition is a reconstituted lyophilized formulation.

In some embodiments, the pharmaceutical composition is a lyophilized composition comprising a residual moisture content between about 1% and about 7%. In some embodiments, the pharmaceutical composition is a lyophilized composition comprising a residual moisture content between about 2% and about 6%. In some embodiments, the pharmaceutical composition is a lyophilized composition comprising a residual moisture content between about 3% and about 4%. In some embodiments, the pharmaceutical composition is a lyophilized composition comprising a residual moisture content about 5%.

In certain aspects, disclosed herein is a method of treating or preventing a disease in a subject, comprising administering to the subject the pharmaceutical composition. In some embodiments, a pharmaceutical composition provided herein is suitable for administration by one, two or more routes of administration (e.g., suitable for suprachoroidal and subretinal administration).

The provided methods are suitable for used in the production of pharmaceutical compositions comprising recombinant AAV encoding a transgene. In some embodiments, provided herein are rAAV viral vectors encoding tripeptidyl peptidase 1 (TPP1) protein. In some embodiments, provided herein are rAAV9-based viral vectors encoding TPP1. In some embodiments, provided herein are rAAV9-based viral vectors encoding CLN2.

In certain aspects, disclosed herein is a method of treating or preventing a disease in a subject, comprising administering to the subject the pharmaceutical composition by intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface).

In certain embodiments, the pharmaceutical composition provided herein is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a desired viscosity that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a desired density that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a desired osmolality that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)). In specific embodiments, the desired osmolality for subretinal administration is 160 430 mOsm/kg H2O. In other specific embodiments, the desired osmolality of suprachoroidal administration is less than 600 mOsm/kg H2O.

In certain embodiments, the pharmaceutical composition has a osmolality of about 100 to 500 mOsm/kg H2O. In certain embodiments, the pharmaceutical composition has a osmolality of about 130 to 470 mOsm/kg H2O. In certain embodiments, the pharmaceutical composition has a osmolality of about 160 to 430 mOsm/kg H2O. In certain embodiments, the pharmaceutical composition has a osmolality of about 200 to 400 mOsm/kg H2O. In certain embodiments, the pharmaceutical composition has a osmolality of about 240 to 340 mOsm/kg H2O. In certain embodiments, the pharmaceutical composition has a osmolality of about 280 to 300 mOsm/kg H2O. In certain embodiments, the pharmaceutical composition has a osmolality of about 295 to 395 mOsm/kg H2O. In certain embodiments, the pharmaceutical composition has a osmolality of less than 600 mOsm/kg H2O. In certain embodiments, the pharmaceutical composition has a osmolality range of 200 mOsm/L to 660 mOsm/L. In certain embodiments, the pharmaceutical composition has a osmolality of about 200 mOsm/L. In certain embodiments, the pharmaceutical composition has a osmolality of about 250 mOsm/L. In certain embodiments, the pharmaceutical composition has a osmolality of about 300 mOsm/L. In certain embodiments, the pharmaceutical composition has a osmolality of about 350 mOsm/L. In certain embodiments, the pharmaceutical composition has a osmolality of about 400 mOsm/L. In certain embodiments, the pharmaceutical composition has a osmolality of about 450 mOsm/L. In certain embodiments, the pharmaceutical composition has a osmolality of about 500 mOsm/L. In certain embodiments, the pharmaceutical composition has a osmolality of about 550 mOsm/L. In certain embodiments, the pharmaceutical composition has a osmolality of about 600 mOsm/L. In certain embodiments, the pharmaceutical composition has a osmolality of about 650 mOsm/L. In certain embodiments, the pharmaceutical composition has a osmolality of about 660 mOsm/L.

The recombinant vector used for delivering the transgene should have a tropism for human retinal cells or photoreceptor cells. Such vectors can include non-replicating recombinant adeno-associated virus vectors (“rAAV”), however, other viral vectors may be used, including but not limited to lentiviral vectors, vaccinia viral vectors, or non-viral expression vectors referred to as “naked DNA” constructs.

In certain embodiments, gene therapy constructs are supplied as a frozen sterile, single use solution of the AAV vector active ingredient in a formulation buffer. In a specific embodiment, the pharmaceutical compositions suitable for subretinal administration comprise a suspension of the recombinant vector in a formulation buffer comprising a physiologically compatible aqueous buffer, a surfactant and optional excipients. In a specific embodiment, the construct is formulated in Dulbecco's phosphate buffered saline and 0.001% poloxamer 188, pH=7.4.

4.3 Gene Therapy

In one aspect, provided herein is a method of treating and/or preventing ocular manifestations of CLN2 Batten disease in a subject comprising administering to the eye of a subject in need thereof a recombinant adeno-associated virus (rAAV) comprising an AAV capsid and a vector genome packaged therein, wherein said vector genome comprises: (a) an AAV 5′ inverted terminal repeat (ITR) sequence; (b) a promoter; (c) a CLN2 coding sequence encoding a human TPP1; and (d) an AAV 3′ ITR. In some embodiments, the rAAV is administered subretinally. In some embodiments, the rAAV is administered suprachoroidally.

In one aspect, provided herein is a method of treating and/or preventing ocular manifestations of CLN2 Batten disease in a subject comprising subretinally administering to a subject in need thereof a recombinant adeno-associated virus (rAAV) comprising an AAV capsid and a vector genome packaged therein, wherein said vector genome comprises: (a) an AAV 5′ inverted terminal repeat (ITR) sequence; (b) a promoter; (c) a CLN2 coding sequence encoding a human TPP1; and (d) an AAV 3′ ITR, wherein the method does not comprise performing a vitrectomy on the eye of said human patient. In certain embodiments, the administering step comprises administering to the subretinal space in the eye of said human subject the recombinant viral vector therapeutic product via the suprachoroidal space in the eye of said human subject. In certain embodiments, the administering step is by the use of a subretinal drug delivery device comprising a catheter that can be inserted and tunneled through the suprachoroidal space toward the posterior pole, where a small needle injects into the subretinal space. In certain embodiments, the administering step comprises inserting and tunneling the catheter of the subretinal drug delivery device through the suprachoroidal space.

In another aspect, provided herein is a method of treating and/or preventing ocular manifestations of CLN2 Batten disease in a subject comprising subretinally administering to a subject in need thereof a recombinant adeno-associated virus (rAAV) comprising an AAV capsid and a vector genome packaged therein, wherein said vector genome comprises: (a) an AAV 5′ inverted terminal repeat (ITR) sequence; (b) a promoter; (c) a CLN2 coding sequence encoding a human TPP1; and (d) an AAV 3′ ITR, wherein the method comprises performing a vitrectomy on the eye of said human patient. In certain embodiments, the vitrectomy is a partial vitrectomy.

In another aspect, provided herein is a method of treating and/or preventing ocular manifestations of CLN2 Batten disease in a subject comprising suprachoroidially administering to a subject in need thereof a recombinant adeno-associated virus (rAAV) comprising an AAV capsid and a vector genome packaged therein, wherein said vector genome comprises: (a) an AAV 5′ inverted terminal repeat (ITR) sequence; (b) a promoter; (c) a CLN2 coding sequence encoding a human TPP1; and (d) an AAV 3′ ITR. In certain embodiments, the administering step is by injecting the recombinant viral vector into the suprachoroidal space using a suprachoroidal drug delivery device. In certain embodiments, the suprachoroidal drug delivery device is a microinjector.

In another aspect, provided herein is a method of treating and/or preventing ocular manifestations of CLN2 Batten disease in a subject comprising administering to the outer surface of the sclera of a subject in need thereof a recombinant adeno-associated virus (rAAV) comprising an AAV capsid and a vector genome packaged therein, wherein said vector genome comprises: (a) an AAV 5′ inverted terminal repeat (ITR) sequence; (b) a promoter; (c) a CLN2 coding sequence encoding a human TPP1; and (d) an AAV 3′ ITR. In certain embodiments, the administering step is by the use of a juxtascleral drug delivery device that comprises a cannula whose tip can be inserted and kept in direct apposition to the scleral surface. In certain embodiments, the administering step comprises inserting and keeping the tip of the cannula in direct apposition to the scleral surface.

In another aspect, provided herein is a method of treating and/or preventing ocular manifestations of CLN2 Batten disease in a subject comprising intravitreally administering to a subject in need thereof a recombinant adeno-associated virus (rAAV) comprising an AAV capsid and a vector genome packaged therein, wherein said vector genome comprises: (a) an AAV 5′ inverted terminal repeat (ITR) sequence; (b) a promoter; (c) a CLN2 coding sequence encoding a human TPP1; and (d) an AAV 3′ ITR. In certain embodiments, the administering step is by injecting the recombinant viral vector into the vitreous cavity using an intravitreal drug delivery device. In certain embodiments, the intravitreal drug delivery device is a microinjector.

In another aspect, provided herein is a method of method of treating and/or preventing ocular manifestations of CLN2 Batten disease in a subject comprising administering to the vitreous cavity of the eye of a subject in need thereof a recombinant adeno-associated virus (rAAV) comprising an AAV capsid and a vector genome packaged therein, wherein said vector genome comprising: (a) an AAV 5′ inverted terminal repeat (ITR) sequence; (b) a promoter; (c) a CLN2 coding sequence encoding a human TPP1; and (d) an AAV 3′ ITR. In certain embodiments, the administering step is by injecting the recombinant viral vector into the vitreous cavity using an intravitreal drug delivery device. In certain embodiments, the intravitreal drug delivery device is a microinjector.

In one aspect, provided herein is a method of subretinal administration accompanied by vitrectomy for treating and/or preventing ocular manifestations of CLN2 Batten disease, comprising administering to the subretinal space in the eye of a human subject in need of treatment an rAAV, wherein the method comprises performing a vitrectomy on the eye of said human patient, and wherein the rAAV comprises an AAV capsid and a vector genome packaged therein, and wherein said vector genome comprising: (a) an AAV 5′ inverted terminal repeat (ITR) sequence; (b) a promoter; (c) a CLN2 coding sequence encoding a human TPP1; and (d) an AAV 3′ ITR. In certain embodiments, the vitrectomy is a partial vitrectomy.

In one aspect, provided herein is a method of subretinal administration for treating and/or preventing ocular manifestations of CLN2 Batten disease, comprising administering to the subretinal space peripheral to the optic disc, fovea and macula located in the back of the eye of a human subject in need of treatment an rAAV, wherein the method does not comprise performing a vitrectomy on the eye of said human patient, and wherein the rAAV comprises an AAV capsid and a vector genome packaged therein, and wherein said vector genome comprising: (a) an AAV 5′ inverted terminal repeat (ITR) sequence; (b) a promoter; (c) a CLN2 coding sequence encoding a human TPP1; and (d) an AAV 3′ ITR. In certain embodiments, the injecting step is by transvitreal injection. In certain embodiments, the method of transvitreal administration results in uniform expression of the therapeutic product throughout the eye (e.g. the expression level at the site of injection varies by less than 5%, 10%, 20%, 30%, 40%, or 50% as compared to the expression level at other areas of the eye). In certain embodiments, the transvitreal injection comprises inserting a sharp needle into the sclera via the superior or inferior side of the eye and passing the sharp needle all the way through the vitreous to inject the recombinant viral vector to the subretinal space on the other side. In certain embodiments, a needle is inserted at the 2 or 10 o'clock position. In certain embodiments, the transvitreal injection comprises inserting a trochar into the sclera and inserting a cannula through the trochar and through the vitreous to inject the recombinant viral vector to the subretinal space on the other side.

In certain embodiments of the methods described herein, the administering step delivers a therapeutically effective amount of the rAAV to the retina of said human subject.

In certain embodiments of the methods described herein, the therapeutically effective amount of the protein encoded by the rAAV genome (e.g., TPP1) is produced by human retinal cells of said human subject.

In certain embodiments of the methods described herein, the therapeutically effective amount of the protein encoded by the rAAV genome (e.g., TPP1) is produced by human photoreceptor cells, horizontal cells, bipolar cells, amacrine cells, retina ganglion cells, and/or retinal pigment epithelial cells in the external limiting membrane of said human subject.

In certain embodiments of the methods described herein, the human photoreceptor cells are cone cells and/or rod cells.

In certain embodiments of the methods described herein, the retina ganglion cells are midget cells, parasol cells, bistratified cells, giant retina ganglion cells, photosensitive ganglion cells, and/or Mûller glia.

In certain embodiments of the methods described herein, the recombinant viral vector is an rAAV vector (e.g., an rAAV8, rAAV2, rAAV9, or rAAV5 vector).

In certain embodiments of the methods described herein, wherein the recombinant viral vector is an rAAV9 vector.

In certain embodiments of the methods described herein, delivering to the eye comprises delivering to the retina, choroid, and/or vitreous humor of the eye.

Methods are described for the administration of a therapeutically effective amount of a recombinant vector (i.e., a recombinant viral vector or a DNA expression construct) to human subjects having pathology of the eye. In some embodiments, the recombinant vector is Construct I, wherein Construct I comprise the following components: an AAV capsid, and a vector genome packaged therein, said vector genome comprising: (a) an AAV 5′ inverted terminal repeat (ITR) sequence; (b) a promoter; (c) a CLN2 coding sequence encoding a human TPP1; and (d) an AAV 3′ ITR. In some embodiments, the 5′ and/or the 3′ ITR are from AAV2. In some embodiments, the promoter is a chicken beta actin promoter. In some embodiments, the methods provided herein are for the treatment of ocular manifestations of Batten disease including, without limitation, vision loss, retinal atrophy, and retinal degeneration.

In particular, methods are described for the administration of a therapeutically effective amount of a recombinant vector (i.e., a recombinant viral vector or a DNA expression construct) to human subjects via one of the following approaches: (1) subretinal administration without vitrectomy (for example, administration to subretinal space via the suprachoroidal space or via peripheral injection), (2) suprachoroidal administration, (3) administration to the outer space of the sclera (i.e., juxtascleral administration); (4) subretinal administration accompanied by vitrectomy; (5) intravitreal administration, and (6) subconjunctival administration.

In certain embodiments, delivery to the subretinal or suprachoroidal space can be performed using the methods and/or devices described and disclosed in International Publication Nos. WO 2016/042162, WO 2017/046358, WO 2017/158365, and WO 2017/158366, each of which is incorporated by reference in its entirety.

4.3.1 Target Patient Populations

In certain embodiments of the methods described herein, the methods provided herein are for the administration to patients having ocular manifestations associated with Batten disease. Ocular manifestations associated with Batten disease include but are not limited to progressive vision loss, retinal atrophy and/or retinal degeneration.

In certain embodiments of the methods described herein, the human subject has a BCVA that is ≤20/20 and ≥20/400. In another specific embodiment, the human subject has a BCVA that is ≤20/63 and ≥20/400.

In some embodiments, the human subject has biallelic CLN2 mutations.

In some embodiments, the human subject has decreased leukocyte TPP1 activity.

In some embodiments, the human subject has clinical signs or symptoms consistent with CLN2 disease (e.g., developmental delay, developmental decline, seizure, vision loss, or other signs symptoms) and/or the subject has an older sibling with confirmed CLN2 diagnosis.

In some embodiments, the human subject has received or is receiving intracerebroventricular Brineura enzyme replacement therapy.

In some embodiments, the human subject has a CRT of about or less than about 210 μm in one or both eyes. In some embodiments, the human subject has a CRT of about or more than about 140 μm in one or both eyes.

In some embodiments, the human subject has, if CLN2 disease was diagnosed in an asymptomatic patient due to family history of CLN2 disease in a sibling, the onset of vision loss in the affected sibling occurs before or around age 84 months.

In some embodiments, the human subject has a CRT of about or less than about 140 μm in one or both eyes and a Weill Cornell Ophthalmic Severity Score of more than 5.

In some embodiments, the human subject does not have significant lens or corneal opacities, glaucoma, amblyopia, and/or gross retinal anatomical abnormality.

In some embodiments, the human subject does not have difference in screening CRT measurement between the right and left eye of more than 10 μm.

In some embodiments, the human subject does not have prior Grade 3 or 4 hypersensitivity reaction, e.g., bronchospasm and hypotension requiring intravenous treatment, cardiac dysfunction, anaphylaxis to intracerebroventricular Brineura infusion.

In some embodiments, the human subject does not have refractive error in the following ranges (myopia ≥−2.00, hyperopia ≥+4.00, astigmatism ≥+1.50) or significant anisometropia.

In some embodiments, the human subject has not had prior intraocular injections of any kind, e.g., off-label intravitreal Brineura.

In some embodiments, the human subject has not had ocular surgery, for example within the prior six months to starting Construct I treatment.

In some embodiments, the human subject has not had a prior bone marrow transplant.

In some embodiments, the human subject has not used the following medications within the 30 days prior to starting Construct I treatment: gemfibrozil, mycophenolate, prednisone or other steroids for the intended purpose of treating NCL (not including asthma indications), flupirtine.

In some embodiments, the human subject does not have contraindications to systemic immunosuppression.

In some embodiments, the human subject does not have mutations in another CLN gene.

In some embodiments, the human subject does not have contraindications to intraocular surgery (e.g., severe coagulopathy).

In certain embodiments, the subject treated in accordance with the methods described herein is female. In certain embodiments, the subject treated in accordance with the methods described herein is male. In certain embodiments, the subject treated in accordance with the methods described herein is a child. In certain embodiments, the subject treated in accordance with the methods described herein is 1 month old, 2 months old, 3 months old, 4 months old, 5 months old, 6 months old, 7 months old, 8 months old, 9 months old, 10 months old, 11 months old, 1 year old, 1.5 years old, 2 years old, 2.5 years old, 3 years old, 3.5 years old, 4 years old, 4.5 years old, or 5 years old. In certain embodiments, the subject treated in accordance with the methods described herein is less than 1.5 months old, 2 months old, 3 months old, 4 months old, 5 months old, 6 months old, 7 months old, 8 months old, 9 months old, 10 months old, 11 months old, 1 year old, 1.5 years old, 2 years old, 2.5 years old, 3 years old, 3.5 years old, 4 years old, 4.5 years old, or less than 5 years old. In another specific embodiment, the subject treated in accordance with the methods described herein is 1-2 months old, 2-3 months old, 3-4 months old, 4-5 months old, 5-6 months old, 6-7 months old, 7-8 months old, 8-9 months old, 9-10 months old, 10-11 months old, 11 months to 1 year old, 1-1.5 years old, 1.5-2 years old, 2-2.5 years old, 2.5-3 years old, 3-3.5 years old, 3.5-4 years old, 4-4.5 years old, or 4.5-5 years old. In another specific embodiment, the subject treated in accordance with the methods described herein is 6 months to 5 years old. In another embodiment, the subject treated in accordance with the methods described herein is a human adult over 18 years old. In some embodiments, the subject treated in accordance with the methods described herein is a human child under 18 years. In some embodiments, the subject treated in accordance with the methods described herein is a human child under 84 months of age.

4.3.2 Dosage and Mode of Administration

In certain embodiments of the method described herein, therapeutically effective doses of the recombinant vector are administered (1) to the subretinal space without vitrectomy (e.g., via the suprachoroidal space or via peripheral injection), (2) to the suprachoroidal space, (3) to the outer space of the sclera (i.e., juxtascleral administration), (4) to the subretinal space via vitrectomy, or (5) to the vitreous cavity, in a volume ranging from 50-100 μl or 100-500 μl, preferably 100-300 μl, and most preferably, 250 μl, depending on the administration method. In certain embodiments, therapeutically effective doses of the recombinant vector are administered suprachoroidally in a volume of 100 μl or less, for example, in a volume of 50-100 μl. In certain embodiments, therapeutically effective doses of the recombinant vector are administered to the outer surface of the sclera (e.g., by a posterior juxtascleral depot procedure) in a volume of 500 μl or less, for example, in a volume of 10-20 μl, 20-50 μl, 50-100 μl, 100-200 μl, 200-300 μl, 300-400 μl, or 400-500 μl. In certain embodiments, therapeutically effective doses of the recombinant vector are administered to the subretinal space via peripheral injection in a volume of 50-100 μl or 100-500 μl, preferably 100-300 μl, and most preferably, 200 μl.

In specific embodiments, therapeutically effective doses of the recombinant vector are administered subretinally in a volume of 200 μL.

In certain embodiment, described herein is an micro volume injector delivery system, which is manufactured by Altaviz (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery.

In certain embodiment, the micro volume injector delivery system can be used for micro volume injector is a micro volume injector with dose guidance and can be used with, for example, a suprachoroidal needle (for example, the Clearside® needle), a subretinal needle, an intravitreal needle, a juxtascleral needle, a subconjunctival needle, and/or intraretinal needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip (for example, the MedOne 38 g needle and the Dorc 41 g needle can be used for subretinal delivery, while the Clearside® needle and the Visionisti OY adaptor can be used for subretinal delivery).

In certain embodiments of the methods described herein, the recombinant vector is administered suprachoroidally (e.g., by suprachoroidal injection). In a specific embodiment, suprachoroidal administration (e.g., an injection into the suprachoroidal space) is performed using a suprachoroidal drug delivery device. Suprachoroidal drug delivery devices are often used in suprachoroidal administration procedures, which involve administration of a drug to the suprachoroidal space of the eye (see, e.g., Hariprasad, 2016, Retinal Physician 13: 20-23; Goldstein, 2014, Retina Today 9(5): 82-87; Baldassarre et al., 2017; each of which is incorporated by reference herein in its entirety). The suprachoroidal drug delivery devices that can be used to deposit the recombinant vector in the suprachoroidal space according to the invention described herein include, but are not limited to, suprachoroidal drug delivery devices manufactured by Clearside® Biomedical, Inc. (see, for example, Hariprasad, 2016, Retinal Physician 13: 20-23) and MedOne suprachoroidal catheters. In another embodiment, the suprachoroidal drug delivery device that can be used in accordance with the methods described herein comprises the micro volume injector delivery system, which is manufactured by Altaviz (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. Currently available technologies for suprachoroidal space (SCS) delivery exist. Preclinically, SC injections have been achieved with scleral flap technique, catheters and standard hypodermic needles, as well as with microneedles. A hollow-bore 750 um-long microneedle (Clearside Biomedical, Inc.) can be inserted at the pars, and has shown promise in clinical trials. A microneedle designed with force-sensing technology can be utilized for SC injections, as described by Chitnis, et al. (Chitnis, G. D., et al. A resistance-sensing mechanical injector for the precise delivery of liquids to target tissue. Nat Biomed Eng 3, 621-631 (2019). https://doi.org/10.1038/s41551-019-0350-2). Oxular Limited is developing a delivery system (Oxulumis) that advances an illuminated cannula in the suprachoroidal space. The Orbit device (Gyroscope) is a specially-designed system enabling cannulation of the suprachoroidal space with a flexible cannula. A microneedle inside the cannula is advanced into the subretinal space to enable targeted dose delivery. Ab interno access to the SCS can also be achieved using micro-stents, which serve as minimally-invasive glaucoma surgery (MIGS) devices. Examples include the CyPass® Micro-Stent (Alcon, Fort Worth, Texas, US) and iStent® (Glaukos), which are surgically implanted to provide a conduit from the anterior chamber to the SCS to drain the aqueous humor without forming a filtering bleb. Other devices contemplated for suprachoroidal delivery include those described in UK Patent Publication No. GB 2531910A and U.S. Pat. No. 10,912,883 B2.

The micro volume injector is a micro volume injector with dose guidance and can be used with, for example, a suprachoroidal needle (for example, the Clearside® needle) or a subretinal needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip (for example, the MedOne 38 g needle and the Dorc 41 g needle can be used for subretinal delivery, while the Clearside® needle and the Visionisti OY adaptor can be used for suprachoroidal delivery). In another embodiment, the suprachoroidal drug delivery device that can be used in accordance with the methods described herein is a tool that comprises a normal length hypodermic needle with an adaptor (and preferably also a needle guide) manufactured by Visionisti OY, which adaptor turns the normal length hypodermic needle into a suprachoroidal needle by controlling the length of the needle tip exposing from the adapter (see, for example, U.S. Design Pat. No. D878,575; and International Patent Application. Publication No. WO/2016/083669) In a specific embodiment, the suprachoroidal drug delivery device is a syringe with a 1 millimeter 30 gauge needle. During an injection using this device, the needle pierces to the base of the sclera and fluid containing drug enters the suprachoroidal space, leading to expansion of the suprachoroidal space. As a result, there is tactile and visual feedback during the injection. Following the injection, the fluid flows posteriorly and absorbs dominantly in the choroid and retina. This results in the production of therapeutic product from all retinal cell layers and choroidal cells. Using this type of device and procedure allows for a quick and easy in-office procedure with low risk of complications. A max volume of 100 μl can be injected into the suprachoroidal space.

In certain embodiments of the methods described herein, the recombinant vector is administered subretinally via vitrectomy. Subretinal administration via vitrectomy is a surgical procedure performed by trained retinal surgeons that involves a vitrectomy with the subject under local anesthesia, and subretinal injection of the gene therapy into the retina (see, e.g., Campochiaro et al., 2017, Hum Gen Ther 28(1):99-111, which is incorporated by reference herein in its entirety).

In certain embodiments of the methods described herein, the recombinant vector is administered subretinally without vitrectomy.

In certain embodiments of the methods described herein, the subretinal administration without vitrectomy is performed via the suprachoroidal space by use of a subretinal drug delivery device. In certain embodiments, the subretinal drug delivery device is a catheter which is inserted and tunneled through the suprachoroidal space around to the back of the eye during a surgical procedure to deliver drug to the subretinal space. This procedure allows the vitreous to remain intact and thus, there are fewer complication risks (less risk of gene therapy egress, and complications such as retinal detachments and macular holes), and without a vitrectomy, the resulting bleb may spread more diffusely allowing more of the surface area of the retina to be transduced with a smaller volume. The risk of induced cataract following this procedure is minimized, which is desirable for younger patients. Moreover, this procedure can deliver bleb under the fovea more safely than the standard transvitreal approach, which is desirable for patients with inherited retinal diseases effecting central vision where the target cells for transduction are in the macula. This procedure is also favorable for patients that have neutralizing antibodies (Nabs) to AAVs present in the systemic circulation which may impact other routes of delivery (such as suprachoroidal and intravitreal). Additionally, this method has shown to create blebs with less egress out the retinotomy site than the standard transvitreal approach. The subretinal drug delivery device originally manufactured by Janssen Pharmaceuticals, Inc. now by Orbit Biomedical Inc. (see, for example, Subretinal Delivery of Cells via the Suprachoroidal Space: Janssen Trial. In: Schwartz et al. (eds) Cellular Therapies for Retinal Disease, Springer, Cham; International Patent Application Publication No. WO 2016/040635 A1) can be used for such purpose.

In another specific embodiment, the subretinal administration without vitrectomy is performed via peripheral injection into the retina (i.e., peripheral to the optic disc, fovea and macula located in the back of the eye). This can be accomplished by transvitreal injection.

In one embodiment, a sharp needle is inserted into the sclera via the superior or inferior side of the eye (e.g., at the 2 or 10 o'clock position) so that the needle passes all the way through the vitreous to inject the retina on the other side. In another embodiment, a trochar is inserted into the sclera to allow a subretinal cannula to be inserted into the eye. The cannula is inserted through the trochar and through the vitreous to the area of desired injection. In either embodiment, the recombinant vector is injected in the subretinal space, forming a bleb containing the recombinant vector on the opposite inner surface of the eye. Successful injection is confirmed by the appearance of a dome shaped retinal detachment/retinal bleb.

A self-illuminating lens may be used as a light source for the transvitreal administration (see e.g., Chalam et al., 2004, Ophthalmic Surgery and Lasers 35: 76-77, which is incorporated by reference herein in its entirety). Alternatively, one or more trochar(s) can be placed for light (or infusion) if desired. In yet another embodiment, an optic fiber chandelier can be utilized via a trocar for visualizing the subretinal injection.

In certain embodiments, delivery to the subretinal or suprachoroidal space can be performed using the methods and/or devices described and disclosed in International Publication Nos. WO 2016/042162, WO 2017/046358, WO 2017/158365, and WO 2017/158366, each of which is incorporated by reference in its entirety.

One, two, or more peripheral injections can be performed to administer the recombinant vector. In this way, one, two, or more blebs containing recombinant vector can be made in the subretinal space peripheral to the optic disc, fovea and macula. Surprisingly, while administration of the recombinant vector is confined to the peripherally injected blebs, expression of the therapeutic product throughout the retina can be detected when using this approach.

In a specific embodiment, the intravitreal administration is performed with a intravitreal drug delivery device that comprises the micro volume injector delivery system, which is manufactured by Altaviz (see, e.g. International Patent Application Publication No. WO 2013/177215), United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. The micro volume injector is a micro volume injector with dose guidance and can be used with, for example, a intravitreal needle. The benefits of using micro volume injector include: (a) more controlled delivery (for example, due to having precision injection flow rate control and dose guidance), (b) single surgeon, single hand, one finger operation; (c) pneumatic drive with 10 increment dosage; (d) divorced from the vitrectomy machine; (e) 400 μL syringe dose; (f) digitally guided delivery; (g) digitally recorded delivery; and (h) agnostic tip (for example, the MedOne 38 g needle and the Dorc 41 g needle can be used for subretinal delivery, while the Clearside® needle and the Visionisti OY adaptor can be used for subretinal delivery).

In certain embodiments, the peripheral injection results in uniform expression of the therapeutic product throughout the eye (e.g. the expression level at the site of injection varies by less than 5%, 10%, 20%, 30%, 40%, or 50% as compared to the expression level at other areas of the eye). The expression of the therapeutic product throughout the eye can be measured by any method known in the art for such a purpose, for example, by whole mount immunofluorescent staining of the eye or retina, or by immunofluorescent staining on frozen ocular sections.

In the event that a transvitreal injection results in loss of the recombinant vector in the vitreous instead of the subretinal space, an optional vitrectomy can be performed to remove the recombinant vector that was injected into the vitreous. A subretinal injection with vitrectomy can then be performed to deliver the 250 μl of recombinant vector into the subretinal space. Alternatively, if some of the injected recombinant vector is deposited into the vitreous and a vitrectomy is not performed to remove the recombinant vector from the vitreous, a catheter lined with immobilized (e.g., covalently bound) anti-AAV antibodies (e.g., anti AAV9 antibodies), can be inserted into the vitreous to capture and remove excess recombinant vector from the vitreous.

In a specific embodiment, the subretinal administration is performed with a subretinal drug delivery device that comprises the micro volume injector delivery system, which is manufactured by Altaviz (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. Micro volume injector is a micro volume injector with dose guidance and can be used with, for example, a subretinal needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip (for example, the MedOne 38 g needle and the Dorc 41 g needle can be used for subretinal delivery, while the Clearside® needle and the Visionisti OY adaptor can be used for suprachoroidal delivery).

In certain embodiments, the recombinant vector is administered to the outer surface of the sclera (for example, by the use of a juxtascleral drug delivery device that comprises a cannula, whose tip can be inserted and kept in direct apposition to the scleral surface). In a specific embodiment, administration to the outer surface of the sclera is performed using a posterior juxtascleral depot procedure, which involves drug being drawn into a blunt-tipped curved cannula and then delivered in direct contact with the outer surface of the sclera without puncturing the eyeball. In particular, following the creation of a small incision to bare sclera, the cannula tip is inserted. The curved portion of the cannula shaft is inserted, keeping the cannula tip in direct apposition to the scleral surface. After complete insertion of the cannula, the drug is slowly injected while gentle pressure is maintained along the top and sides of the cannula shaft with sterile cotton swabs. This method of delivery avoids the risk of intraocular infection and retinal detachment, side effects commonly associated with injecting therapeutic agents directly into the eye.

In a specific embodiment, the juxtascleral administration is performed with a juxtascleral drug delivery device that comprises the micro volume injector delivery system, which is manufactured by Altaviz (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. Micro Volume Injector is a micro volume injector with dose guidance and can be used with, for example, a juxtascleral needle. The benefits of using micro volume injector include: (a) more controlled delivery (for example, due to having precision injection flow rate control and dose guidance), (b) single surgeon, single hand, one finger operation; (c) pneumatic drive with 10 increment dosage; (d) divorced from the vitrectomy machine; (e) 400 μL syringe dose; (f) digitally guided delivery; (g) digitally recorded delivery; and (h) agnostic tip.

In certain embodiments, an infrared thermal camera can be used to detect changes in the thermal profile of the ocular surface after the administering of a solution which is cooler than body temperature to detect changes in the thermal profile of the ocular surface that allows for visualization of the spread of the solution, e.g., within the SCS, and can potentially determine whether the administration was successfully completed. This is because in certain embodiments the formulation containing the recombinant vector to be administered is initially frozen, brought to room temperature (68-72° F.), and thawed for a short period of time (e.g., at least 30 minutes) before administration, and thus the formulation is colder than the human eye (about 92° F.) (and sometimes even colder than room temperature) at the time of injection. The drug product is typically used within 4 hours of thaw and the warmest the solution would be is room temperature. In a preferred embodiment, the procedure is videoed with infrared video.

Infrared thermal cameras can detect small changes in temperature. They capture infrared energy through a lens and convert the energy into an electronic signal. The infrared light is focused onto an infrared sensor array which converts the energy into a thermal image. The infrared thermal camera can be used for any method of administration to the eye, including any administration route described herein, for example, suprachoroidal administration, subretinal administration, subconjunctival administration, intravitreal administration, or administration with the use of a slow infusion catheter in to the suprachoroidal space. In a specific embodiment, the infrared thermal camera is an FLIR T530 infrared thermal camera. The FLIR T530 infrared thermal camera can capture slight temperature differences with an accuracy of ±3.6° F. The camera has an infrared resolution of 76,800 pixels. The camera also utilizes a 24° lens capturing a smaller field of view. A smaller field of view in combination with a high infrared resolution contributes to more detailed thermal profiles of what the operator is imaging. However, other infrared camera can be used that have different abilities and accuracy for capturing slight temperature changes, with different infrared resolutions, and/or with different degrees of lens.

In a specific embodiment, the infrared thermal camera is an FLIR T420 infrared thermal camera. In a specific embodiment, the infrared thermal camera is an FLIR T440 infrared thermal camera. In a specific embodiment, the infrared thermal camera is an Fluke Ti400 infrared thermal camera. In a specific embodiment, the infrared thermal camera is an FLIRE60 infrared thermal camera. In a specific embodiment, the infrared resolution of the infrared thermal camera is equal to or greater than 75,000 pixels. In a specific embodiment, the thermal sensitivity of the infrared thermal camera is equal to or smaller than 0.05° C. at 30° C. In a specific embodiment, the field of view (FOV) of the infrared thermal camera is equal to or lower than 25°×25°.

In certain embodiments, an iron filer is used with the infrared thermal camera to detect changes in the thermal profile of the ocular surface. In a preferred embodiment, the use of an iron filter is able to a generate pseudo-color image, wherein the warmest or high temperature parts are colored white, intermediate temperatures are reds and yellows, and the coolest or low temperature parts are black. In certain embodiments, other types of filters can also be used to generate pseudo-color images of the thermal profile.

The thermal profile for each administration method can be different. For example, in one embodiment, a successful suprachoroidal injection can be characterized by: (a) a slow, wide radial spread of the dark color, (b) very dark color at the beginning, and (c) a gradual change of injectate to lighter color, i.e., a temperature gradient noted by a lighter color. In one embodiment, an unsuccessful suprachoroidal injection can be characterized by: (a) no spread of the dark color, and (b) a minor change in color localized to the injection site without any distribution. In certain embodiments, the small localized temperature drop is result from cannula (low temperature) touching the ocular tissues (high temperature). In one embodiment, a successful intravitreal injection can be characterized by: (a) no spread of the dark color, (b) an initial change to very dark color localized to the injection site, and (c) a gradual and uniform change of the entire eye to darker color. In one embodiment, an extraocular efflux can be characterized by: (a) quick flowing streams on outside on the exterior surface of the eye, (b) very dark color at the beginning, and (c) a quick change to lighter color.

Because the therapeutic product is continuously produced (under the control of a constitutive promoter or induced by hypoxic conditions when using an hypoxia-inducible promoter), maintenance of lower concentrations can be effective. Vitreous humour concentrations can be measured directly in patient samples of fluid collected from the vitreous humour or the anterior chamber, or estimated and/or monitored by measuring the patient's serum concentrations of the therapeutic product—the ratio of systemic to vitreal exposure to the therapeutic product is about 1:90,000. (E.g., see, vitreous humor and serum concentrations of ranibizumab reported in Xu L, et al., 2013, Invest. Opthal. Vis. Sci. 54: 1616-1624, at p. 1621 and Table 5 at p. 1623, which is incorporated by reference herein in its entirety).

In certain embodiments, dosages are measured by genome copies per ml or the number of genome copies administered to the eye of the patient (e.g., administered suprachoroidally, subretinally, intravitreally, juxtasclerally, subconjunctivally, and/or intraretinally. In certain embodiments, 1×10⁹ genome copies per ml to 1×10¹⁵ genome copies per ml are administered. In a specific embodiment, 1×10⁹ genome copies per ml to 1×10¹⁰ genome copies per ml are administered. In another specific embodiment, 1×10¹⁰ genome copies per ml to 1×10¹¹ genome copies per ml are administered. In another specific embodiment, 1×10¹⁰ to 5×10¹¹ genome copies are administered. In another specific embodiment, 1×10¹¹ genome copies per ml to 1×10¹² genome copies per ml are administered. In another specific embodiment, 1×10¹² genome copies per ml to 1×10¹³ genome copies per ml are administered. In another specific embodiment, 1×10¹³ genome copies per ml to 1×10¹⁴ genome copies per ml are administered. In another specific embodiment, 1×10¹⁴ genome copies per ml to 1×10¹⁵ genome copies per ml are administered. In another specific embodiment, about 1×10⁹ genome copies per ml are administered. In another specific embodiment, about 1×10¹⁰ genome copies per ml are administered. In another specific embodiment, about 1×10¹¹ genome copies per ml are administered. In another specific embodiment, about 1×10¹² genome copies per ml are administered. In another specific embodiment, about 1×10¹³ genome copies per ml are administered. In another specific embodiment, about 1×10¹⁴ genome copies per ml are administered. In another specific embodiment, about 1×10¹⁵ genome copies per ml are administered. In certain embodiments, 1×10⁹ to 1×10¹⁵ genome copies are administered. In a specific embodiment, 1×10⁹ to 1×10¹⁰ genome copies are administered. In another specific embodiment, 1×10¹⁰ to 1×10¹¹ genome copies are administered. In another specific embodiment, 1×10¹⁰ to 5×10¹¹ genome copies are administered. In another specific embodiment, 1×10¹¹ to 1×10¹² genome copies are administered. In another specific embodiment, 1×10¹² to 1×10¹³ genome copies are administered. In another specific embodiment, 1×10¹³ to 1×10¹⁴ genome copies are administered. In another specific embodiment, 1×10¹³ to 1×10¹⁴ genome copies are administered. In another specific embodiment, 1×10¹⁴ to 1×10¹⁵ genome copies are administered. In another specific embodiment, about 1×10⁹ genome copies are administered. In another specific embodiment, about 1×10¹⁰ genome copies are administered. In another specific embodiment, about 1×10¹¹ genome copies are administered. In another specific embodiment, about 1×10¹² genome copies are administered. In another specific embodiment, about 1×10¹³ genome copies are administered. In another specific embodiment, about 1×10¹⁴ genome copies are administered. In another specific embodiment, about 1×10¹⁵ genome copies are administered. In certain embodiments, about 3.0×10¹³ genome copies per eye are administered. In certain embodiments, up to 3.0×10¹³ genome copies per eye are administered.

In certain embodiments, about 2.0×10¹⁰ genome copies per eye are administered. In certain embodiments, about 6.0×10¹⁰ genome copies per eye are administered.

In certain embodiments, about 1.0×10¹⁰ to 2.0×10¹⁰ genome copies per eye are administered by subretinal injection. In certain embodiments, about 2.0×10¹⁰ to 3.0×10¹⁰ genome copies per eye are administered by subretinal injection. In certain embodiments, about 3.0×10¹⁰ to 4.0×10¹⁰ genome copies per eye are administered by subretinal injection. In certain embodiments, about 4.0×10¹⁰ to 5.0×10¹⁰ genome copies per eye are administered by subretinal injection. In certain embodiments, about 5.0×10¹⁰ to 6.0×10¹⁰ genome copies per eye are administered by subretinal injection. In certain embodiments, about 6.0×10¹⁰ to 7.0×10¹⁰ genome copies per eye are administered by subretinal injection. In certain embodiments, about 7.0×10¹⁰ to 8.0×10¹⁰ genome copies per eye are administered by subretinal injection. In certain embodiments, about 8.0×10¹⁰ to 9.0×10¹⁰ genome copies per eye are administered by subretinal injection. In certain embodiments, about 9.0×10¹⁰ to 1.0×10¹¹ genome copies per eye are administered by subretinal injection. In certain embodiments, about 1.0×10¹¹ to 2.0×10¹¹ genome copies per eye are administered by subretinal injection.

In certain specific embodiments, about 2.0×10¹⁰ genome copies per eye are administered by subretinal injection. In certain specific embodiments, about 6.0×10¹⁰ genome copies per eye are administered by subretinal injection. In some embodiments, the injection volume of a subretinal injection is 200 μL.

In certain embodiments, about 2×10¹⁰ to 3×10¹⁰ genome copies per eye are administered by suprachoroidal injection. In certain embodiments, about 3×10¹⁰ to 4×10¹⁰ genome copies per eye are administered by suprachoroidal injection. In certain embodiments, about 4×10¹⁰ to 5×10¹⁰ genome copies per eye are administered by suprachoroidal injection.

In certain embodiments, about 5×10¹⁰ to 6×10¹⁰ genome copies per eye are administered by suprachoroidal injection. In certain embodiments, about 6×10¹⁰ to 7×10¹⁰ genome copies per eye are administered by suprachoroidal injection. In certain embodiments, about 7×10¹⁰ to 8×10¹⁰ genome copies per eye are administered by suprachoroidal injection. In certain embodiments, about 8×10¹⁰ to 9×10¹⁰ genome copies per eye are administered by suprachoroidal injection. In certain embodiments, about 9×10¹⁰ to 1×10¹¹ genome copies per eye are administered by suprachoroidal injection. In certain embodiments, about 1×10¹¹ to 2×10¹¹ genome copies per eye are administered by suprachoroidal injection. In certain embodiments, about 2×10¹¹ to 3×10¹¹ genome copies per eye are administered by suprachoroidal injection. In certain embodiments, about 3×10¹¹ to 4×10¹¹ genome copies per eye are administered by suprachoroidal injection. In certain embodiments, about 4×10¹¹ to 5×10¹¹ genome copies per eye are administered by suprachoroidal injection. In certain embodiments, about 5×10¹¹ to 6×10¹¹ genome copies per eye are administered by suprachoroidal injection. In certain embodiments, about 6×10¹¹ to 7×10¹¹ genome copies per eye are administered by suprachoroidal injection. In certain embodiments, about 7×10¹¹ to 8×10¹¹ genome copies per eye are administered by suprachoroidal injection. In certain embodiments, about 8×10¹¹ to 9×10¹¹ genome copies per eye are administered by suprachoroidal injection. In certain embodiments, about 9×10¹¹ to 1×10¹² genome copies per eye are administered by suprachoroidal injection.

In certain embodiments, about 2×10¹⁰ genome copies per eye are administered by suprachoroidal injection. In certain embodiments, about 3×10¹⁰ genome copies per eye are administered by suprachoroidal injection. In certain embodiments, about 4×10¹⁰ genome copies per eye are administered by suprachoroidal injection. In certain embodiments, about 5×10¹⁰ genome copies per eye are administered by suprachoroidal injection. In certain embodiments, about 6×10¹⁰ genome copies per eye are administered by suprachoroidal injection. In certain embodiments, about 7×10¹⁰ genome copies per eye are administered by suprachoroidal injection. In certain embodiments, about 8×10¹⁰ genome copies per eye are administered by suprachoroidal injection. In certain embodiments, about 9×10¹⁰ genome copies per eye are administered by suprachoroidal injection. In certain embodiments, about 1×10¹¹ genome copies per eye are administered by suprachoroidal injection. In certain embodiments, about 1.5×10¹¹ genome copies per eye are administered by suprachoroidal injection. In certain embodiments, about 2×10¹¹ genome copies per eye are administered by suprachoroidal injection. In certain embodiments, about 2.5×10¹¹ genome copies per eye are administered by suprachoroidal injection. In certain embodiments, about 3×10¹¹ genome copies per eye are administered by suprachoroidal injection.

In certain embodiments, a single suprachoroidal injection is administered. In certain embodiments, a double suprachoroidal injection is administered. In certain embodiments, the injection volume for a suprachoroidal injection is 100 μl.

As used herein and unless otherwise specified, the term “about” means within plus or minus 10% of a given value or range. In certain embodiments, the term “about” encompasses the exact number recited.

4.3.3 Sampling and Monitoring of Efficacy

In certain embodiments, when the human subject has disease manifestations in both the CNS and the eye (for example, when the human subject has a Batten disease), the method provided herein comprises administering a recombinant vector described herein (i.e., a recombinant viral vector or a DNA expression construct) to the human subject via both a central nervous system (CNS) delivery route and an ocular delivery route (for example, an ocular delivery route described herein). In certain embodiments, the ocular delivery route is selected from one of the following: (1) subretinal administration without vitrectomy (for example, administration to subretinal space via the suprachoroidal space or via peripheral injection), (2) suprachoroidal administration, (3) administration to the outer space of the sclera (i.e., juxtascleral administration); (4) subretinal administration accompanied by vitrectomy; (5) intravitreal administration, and (6) intravitreal administration. In certain embodiments, the CNS delivery route is selected from one of the following: intracerebroventricular (ICV) delivery, intracisternal (IC) delivery, or intrathecal-lumbar (IT-L) delivery.

Effects of the methods provided herein on visual deficits may be measured by BCVA (Best-Corrected Visual Acuity), intraocular pressure, slit lamp biomicroscopy, and/or indirect ophthalmoscopy.

In specific embodiments, effects of the methods provided herein on visual deficits may be measured by whether the human patient's eye that is treated by a method described herein achieves BCVA of greater than 43 letters post-treatment (e.g., 46-50 weeks or 98-102 weeks post-treatment). A BCVA of 43 letters corresponds to 20/160 approximate Snellen equivalent. In a specific embodiment, the human patient's eye that is treated by a method described herein achieves BCVA of greater than 43 letters post-treatment (e.g., 46-50 weeks or 98-102 weeks post-treatment).

In specific embodiments, effects of the methods provided herein on visual deficits may be measured by whether the human patient's eye that is treated by a method described herein achieves BCVA of greater than 84 letters post-treatment (e.g., 46-50 weeks or 98-102 weeks post-treatment). A BCVA of 84 letters corresponds to 20/20 approximate Snellen equivalent. In a specific embodiment, the human patient's eye that is treated by a method described herein achieves BCVA of greater than 84 letters post-treatment (e.g., 46-50 weeks or 98-102 weeks post-treatment).

Effects of the methods provided herein on physical changes to eye/retina may be measured by SD-OCT (SD-Optical Coherence Tomography).

Efficacy may be monitored as measured by electroretinography (ERG).

Effects of the methods provided herein may be monitored by measuring signs of vision loss, infection, inflammation and other safety events, including retinal detachment.

Retinal thickness may be monitored to determine efficacy of the methods provided herein. Without being bound by any particular theory, thickness of the retina may be used as a clinical readout, wherein the greater reduction in retinal thickness or the longer period of time before thickening of the retina, the more efficacious the treatment. Retinal function may be determined, for example, by ERG. ERG is a non-invasive electrophysiologic test of retinal function, approved by the FDA for use in humans, which examines the light sensitive cells of the eye (the rods and cones), and their connecting ganglion cells, in particular, their response to a flash stimulation. Retinal thickness may be determined, for example, by SD-OCT. SD-OCT is a three-dimensional imaging technology which uses low-coherence interferometry to determine the echo time delay and magnitude of backscattered light reflected off an object of interest. OCT can be used to scan the layers of a tissue sample (e.g., the retina) with 3 to 15 μm axial resolution, and SD-OCT improves axial resolution and scan speed over previous forms of the technology (Schuman, 2008, Trans. Am. Opthamol. Soc. 106:426-458).

Effects of the methods provided herein may also be measured by a change from baseline in National Eye Institute Visual Functioning Questionnaire, the Rasch-scored version (NEI-VFQ-28-R) (composite score; activity limitation domain score; and socio-emotional functioning domain score). Effects of the methods provided herein may also be measured by a change from baseline in National Eye Institute Visual Functioning Questionnaire 25-item version (NEI-VFQ-25) (composite score and mental health subscale score). Effects of the methods provided herein may also be measured by a change from baseline in Macular Disease Treatment Satisfaction Questionnaire (MacTSQ) (composite score; safety, efficacy, and discomfort domain score; and information provision and convenience domain score).

In specific embodiments, the efficacy of a method described herein is reflected by an improvement in vision at about 4 weeks, 12 weeks, 6 months, 12 months, 24 months, 36 months, or at other desired timepoints. In a specific embodiment, the improvement in vision is characterized by an increase in BCVA, for example, an increase by 1 letter, 2 letters, 3 letters, 4 letters, 5 letters, 6 letters, 7 letters, 8 letters, 9 letters, 10 letters, 11 letters, or 12 letters, or more. In a specific embodiment, the improvement in vision is characterized by a 5%, 10%, 15%, 20%, 30%, 40%, 50% or more increase in visual acuity from baseline.

In specific embodiments, the efficacy of a method described herein is reflected by an reduction in central retinal thickness (CRT) or outer nuclear layer thickness (OLN) at about 4 weeks, 12 weeks, 6 months, 12 months, 24 months, 36 months, or at other desired timepoint, for example, a 5%, 10%, 15%, 20%, 30%, 40%, 50% or more decrease in central retinal thickness from baseline.

In s specific embodiments, there is no inflammation in the eye after treatment or little inflammation in the eye after treatment (for example, an increase in the level of inflammation by 10%, 5%, 2%, 1% or less from baseline).

Effects of the methods provided herein on visual deficits may be measured by OptoKinetic Nystagmus (OKN).

Without being bound by theory, this visual acuity screening uses the principles of the OKN involuntary reflex to objectively assess whether a patient's eyes can follow a moving target. By using OKN, no verbal communication is needed between the tester and the patient. As such, OKN can be used to measure visual acuity in pre-verbal and/or non-verbal patients. In certain embodiments, OKN is used to measure visual acuity in patients that are 1 month old, 2 months old, 3 months old, 4 months old, 5 months old, 6 months old, 7 months old, 8 months old, 9 months old, 10 months old, 11 months old, 1 year old, 1.5 years old, 2 years old, 2.5 years old, 3 years old, 3.5 years old, 4 years old, 4.5 years old, or 5 years old. In certain embodiments, an iPad is used to measure visual acuity through detection of the OKN reflex when a patient is looking at movement on the iPad.

Without being bound by theory, this visual acuity screening uses the principles of the OKN involuntary reflex to objectively assess whether a patient's eyes can follow a moving target. By using OKN, no verbal communication is needed between the tester and the patient. As such, OKN can be used to measure visual acuity in pre-verbal and/or non-verbal patients. In certain embodiments, OKN is used to measure visual acuity in patients that are less than 1.5 months old, 2 months old, 3 months old, 4 months old, 5 months old, 6 months old, 7 months old, 8 months old, 9 months old, 10 months old, 11 months old, 1 year old, 1.5 years old, 2 years old, 2.5 years old, 3 years old, 3.5 years old, 4 years old, 4.5 years old, or 5 years old. In another specific embodiment, OKN is used to measure visual acuity in patients that are 1-2 months old, 2-3 months old, 3-4 months old, 4-5 months old, 5-6 months old, 6-7 months old, 7-8 months old, 8-9 months old, 9-10 months old, 10-11 months old, 11 months to 1 year old, 1-1.5 years old, 1.5-2 years old, 2-2.5 years old, 2.5-3 years old, 3-3.5 years old, 3.5-4 years old, 4-4.5 years old, or 4.5-5 years old. In another specific embodiment, OKN is used to measure visual acuity in patients that are 6 months to 5 years old. In certain embodiments, an iPad is used to measure visual acuity through detection of the OKN reflex when a patient is looking at movement on the iPad.

In certain embodiments, visual acuity is assessed in a patient presenting with Batten-CLN2-associated vision loss by measuring OKN before the patient has been treated with an AAV, preferably AAV9, encoding Tripeptidyl-Peptidase 1 (TPP1). Specifically, the patient presenting with Batten-CLN2-associated vision loss is at the age, and/or within the age range described above. In certain embodiments, visual acuity assessed in a patient up to 5 years old presenting with Batten-CLN2-associated vision loss by measuring OKN after the patient has been treated with an AAV, preferably AAV9, encoding Tripeptidyl-Peptidase 1. In certain embodiments, a visual acuity assessment based on OKN determines that visual acuity does not decrease after treatment with AAV gene therapy. In certain embodiments, a visual acuity assessment based on OKN determines that visual acuity improves in a patient after treatment with AAV gene therapy by 5%, 10%, 15%, 20%, 25%, 30%, 35%, 40% 45%, 50%, 55%, 60%, 65%, 70%, 75%, 80%, 85%, 90%, 95% or 100%. In certain embodiments, a visual acuity assessment based on OKN determines that visual acuity does not further deteriorate in a patient after treatment with AAV gene therapy.

In certain embodiments, visual acuity is assessed in a patient presenting with Batten-CLN2-associated vision loss by measuring OKN before the patient has been treated with an AAV, preferably AAV9, encoding TPP1. Specifically, the patient presenting with Batten-CLN2-associated vision loss is at the age, and/or within the age range described above. In certain embodiments, visual acuity is assessed in a patient up to 5 years old presenting with Batten-CLN2-associated vision loss by measuring OKN after the patient has been treated with an AAV, preferably AAV9, encoding Tripeptidyl-Peptidase 1. In certain embodiments, a visual acuity assessment based on OKN determines that visual acuity does not decrease after treatment with AAV gene therapy. In certain embodiments, a visual acuity assessment based on OKN determines that visual acuity improves in a patient after treatment with AAV gene therapy by at least 5%, 10%, 15%, 20%, 25%, 30%, 35%, 40% 45%, 50%, 55%, 60%, 65%, 70%, 75%, 80%, 85%, 90%, 95% or at least 100%. In certain embodiments, a visual acuity assessment based on OKN determines that visual acuity does not further deteriorate in a patient after treatment with AAV gene therapy.

If the human patient is a child, visual function can be assessed using an optokinetic nystagmus (OKN)-based approach or a modified OKN-based approach.

Effects of the methods provided herein may also be measured by a change in pupillary light reflex as measured by pupillometry over time, for example, a change in pupillary light reflex a 5%, 10%, 15%, 20%, 30%, 40%, 50%, 60%, 70%, 80%, 90% or more change in pupillary light reflex from baseline at about 1-2, 2-4, 4-6, 8-10, 10-12, 12-14, 12-16, 16-18, 18-20, 20-22, 22-24 months or at about 1, 3, 6, 9, 12, 15, 18, 21, or 24 months from baseline.

Effects of the methods provided herein may also be measured by a change in macular thickness and/or volume as measured by SD-OCT over time, for example, a 5%, 10%, 15%, 20%, 30%, 40%, 50%, 60%, 70%, 80%, 90% or more change in macular thickness and/or volume from baseline at about 1-2, 2-4, 4-6, 8-10, 10-12, 12-14, 12-16, 16-18, 18-20, 20-22, 22-24 months or at about 1, 3, 6, 9, 12, 15, 18, 21, or 24 months from baseline.

Effects of the methods provided herein may also be measured by a change in time to accelerated decline phase of retinal degradation (e.g., reaching 210 μm CRT), for example, a 5%, 10%, 15%, 20%, 30%, 40%, 50%, 60%, 70%, 80%, 90% or more change in time to accelerated decline phase of retinal degradation, or in the time to 30 μm loss in CRT or to CRT of less than about 110 μm, e.g., about 1-2, 2-4, 4-6, 8-10, 10-12, 12-14, 12-16, 16-18, 18-20, 20-22, 22-24 months or about 1, 3, 6, 9, 12, 15, 18, 21, or 24 months to 30 μm loss in CRT or to CRT of less than about 110 μm.

Effects of the methods provided herein may also be measured by a change in SD-OCT anatomical markers, e.g., parafoveal ellipsoid zone, ELM, RNFL, full retinal thickness, the inner nuclear layer, the outer nuclear layer (ONL), the photoreceptor (PR) plus the retinal pigment epithelium (RPE), the outer segment plus the RPE (OS+RPE), and/or the ellipsoid zone (EZ) over time, for example, a 5%, 10%, 15%, 20%, 30%, 40%, 50%, 60%, 70%, 80%, 90% or more change in SD-OCT anatomical markers, e.g., parafoveal ellipsoid zone, ELM, RNFL full retinal thickness, the inner nuclear layer, the outer nuclear layer (ONL), the photoreceptor (PR) plus the retinal pigment epithelium (RPE), the outer segment plus the RPE (OS+RPE), and/or the ellipsoid zone (EZ) from baseline at about 1-2, 2-4, 4-6, 8-10, 10-12, 12-14, 12-16, 16-18, 18-20, 20-22, 22-24 months or at about 1, 3, 6, 9, 12, 15, 18, 21, or 24 months from baseline.

Effects of the methods provided herein may also be measured by a change in fundus appearance on fundus photography over time as measured by the modified WCBS over time, for example, a 5%, 10%, 15%, 20%, 30%, 40%, 50%, 60%, 70%, 80%, 90% or more change in fundus appearance on fundus photography over time as measured by the modified WCBS from baseline at about 1-2, 2-4, 4-6, 8-10, 10-12, 12-14, 12-16, 16-18, 18-20, 20-22, 22-24 months or at about 1, 3, 6, 9, 12, 15, 18, 21, or 24 months from baseline.

Effects of the methods provided herein may also be measured by a change in caregiver-reported visual outcome over time as assessed using PedEyeQ over time, for example, a 5%, 10%, 15%, 20%, 30%, 40%, 50%, 60%, 70%, 80%, 90% or more change in caregiver-reported visual outcome over time as assessed using PedEyeQ over time as measured by the modified WCBS from baseline at about 1-2, 2-4, 4-6, 8-10, 10-12, 12-14, 12-16, 16-18, 18-20, 20-22, 22-24 months or at about 1, 3, 6, 9, 12, 15, 18, 21, or 24 months from baseline.

Effects of the methods provided herein may also be measured by a change in adaptive behaviors based on cognitive ability over time as assessed using the VABs III and MSEL-Visual Reception over time, for example, a 5%, 10%, 15%, 20%, 30%, 40%, 50%, 60%, 70%, 80%, 90% or more change in adaptive behaviors based on cognitive ability over time as assessed using the VABs III and MSEL-Visual Reception over time from baseline at about 1-2, 2-4, 4-6, 8-10, 10-12, 12-14, 12-16, 16-18, 18-20, 20-22, 22-24 months or at about 1, 3, 6, 9, 12, 15, 18, 21, or 24 months from baseline.

Effects of the methods provided herein may also be measured by a change in the Hamburg Scale Motor Function score using the treated eye and control eye over time, for example, a 5%, 10%, 15%, 20%, 30%, 40%, 50%, 60%, 70%, 80%, 90% or more change in the Hamburg Scale Motor Function score using the treated eye and control eye over time as measured by the modified WCBS from baseline at about 1-2, 2-4, 4-6, 8-10, 10-12, 12-14, 12-16, 16-18, 18-20, 20-22, 22-24 months or at about 1, 3, 6, 9, 12, 15, 18, 21, or 24 months from baseline.

In another aspect, vector shedding may be determined for example by measuring vector DNA in biological fluids such as tears, serum or urine using quantitative polymerase chain reaction. In some embodiments, no vector gene copies are detectable in a biological fluid (e.g., tears, serum or urine) at any time point after administration of the vector. In some embodiments, less than 1000, less than 500, less than 100, less than 50 or less than 10 vector gene copies/5 μL are detectable by quantitative polymerase chain reaction in a biological fluid (e.g., tears, serum or urine) at any point after administration. In specific embodiments, 210 vector gene copies/5 μL or less are detectable in serum. In some embodiments, less than 1000, less than 500, less than 100, less than 50 or less than 10 vector gene copies/5 μL are detectable by quantitative polymerase chain reaction in a biological fluid (e.g., tears, serum or urine) by 1, 2, 3, 4, 5, 6, 7, 8, 9, 10, 11, 12, 13 or 14 weeks after administration. In specific embodiments, no vector gene copies are detectable in serum by week 14 after administration of the vector.

In another aspect, expression levels of the transgene (e.g., the transgene encoded by Construct I, or TPP1) may be monitored by measuring the transgene product in the serum and/or the CSF of a subject to whom Construct I has been administered. Expression levels of the transgene product (e.g., TPP1) may also be monitored in the eye (e.g., the aqueous humour and/or the vitreous humour) of a subject to whom Construct I has been administered. Transgene expression may be measured by any suitable assay known in the art, including, without limitation, Western Blotting, electrochemiluminescent (ECL) immunoassays implemented using the Meso Scale Discovery (MSD) platform, and ELISA.

In some embodiments, expression levels of TPP1 in the eye may vary between different areas of the eye, e.g., high levels of vector DNA may be detected in the retina and choroid/RPE at the area of the bleb (temporal, due to the injection) as well as the superior, inferior and nasal quadrants, while low levels may be detected in the optic nerve, optic chiasm and occipital lobe. In some embodiments, an rAAV provided herein (e.g., Construct I) demonstrates limited anterograde tropism along the visual pathway.

In some embodiments, administration of an rAAV provided herein (e.g., Construct I) to a subject results in detectable (e.g., detectable using a method described herein, or a method known in the art) TPP1 expression levels in the vitreous humour and/or the aqueous humour of the eye of the subject within about 3 months, about 6 months, about 9 months, about 12 months, about 15 months, about 18 months, about 21 months, or about 24 months of administration of the rAAV to the subject, wherein the levels of TPP1 expression in vitreous humour and/or the aqueous humour were undetectable prior to administration of the rAAV. In some embodiments, administration of an rAAV provided herein (e.g., Construct I) to a subject results in detectable (e.g., detectable using a method described herein, or a method known in the art) TPP1 expression levels in the vitreous humour and/or the aqueous humour of the eye of the subject within about 3 months, about 6 months, about 9 months, about 12 months, about 15 months, about 18 months, about 21 months, or about 24 months of administration of the rAAV to the subject, wherein the levels of TPP1 expression in the vitreous humour and/or the aqueous humour were undetectable prior to administration of the rAAV, and wherein TPP1 expression levels in serum of the subject remain undetectable (e.g., undetectable using a method described herein, or a method known in the art). In some embodiments, TPP1 is detectable or undetectable using an immunofluorescence assay. In some embodiments, TPP1 is detectable or undetectable using an immunostaining assay.

In some embodiments, administration of an rAAV provided herein (e.g., Construct I) to a subject results in an increase in TPP1 expression levels (e.g., an increase of about 100-fold to about 500-fold, about 500-fold to about 1000-fold, about 1000-fold to about 1500-fold, about 1500-fold to about 2000-fold, about 2000-fold to about 2500-fold, about 2500-fold to about 3000-fold, about 3000-fold to about 4000-fold, about 4000-fold to about 5000-fold, about 5000-fold to about 10,000-fold, about 10,000-fold to about 15,000-fold, about 15,000-fold to about 20,000-fold, or more than about 20,000-fold) in the vitreous humour and/or the aqueous humour of the eye of the subject within about 3 months, about 6 months, about 9 months, about 12 months, about 15 months, about 18 months, about 21 months, or about 24 months of administration of the rAAV to the subject compared to TPP1 expression levels prior to administration of the rAAV. In some embodiments, administration of an rAAV provided herein (e.g., Construct I) to a subject results in an increase in TPP1 expression levels (e.g., an increase of about 100-fold to about 500-fold, about 500-fold to about 1000-fold, about 1000-fold to about 1500-fold, about 1500-fold to about 2000-fold, about 2000-fold to about 2500-fold, about 2500-fold to about 3000-fold, about 3000-fold to about 4000-fold, about 4000-fold to about 5000-fold, about 5000-fold to about 10,000-fold, about 10,000-fold to about 15,000-fold, about 15,000-fold to about 20,000-fold, or more than about 20,000-fold) in the vitreous humour and/or the aqueous humour of the eye of the subject within about 3 months, about 6 months, about 9 months, about 12 months, about 15 months, about 18 months, about 21 months, or about 24 months of administration of the rAAV to the subject compared to TPP1 expression levels prior to administration of the rAAV, wherein TPP1 expression levels in serum of the subject remain about constant (e.g., remain within about 10%) compared to the TPP1 expression levels prior to administration. In some embodiments, TPP1 expression is measured using an immunofluorescence assay. In some embodiments, TPP1 expression is detectable or undetectable using an immunostaining assay.

In some embodiments, administration of an rAAV provided herein (e.g., Construct I) to a subject results in TPP1 expression in the retina of the subject, e.g., expression in the retinal pigment epithelium and/or the photoreceptor outer segments, or a proportion thereof, or expression across the entire depths of the retina. In some embodiments, TPP1 expression may be confined to neurons.

Transgene product levels can be measured in patient samples of the vitreous humour and/or aqueous from the anterior chamber of the treated eye. Alternatively, vitreous humour concentrations can be estimated and/or monitored by measuring the patient's serum concentrations of the transgene product—the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54: 1616-1624, at p. 1621 and Table 5 at p. 1623, which is incorporated by reference herein in its entirety).

Immunogenicity to Construct I may be evaluated, assessed as anti-transgene product antibodies (aqueous humor, serum, and CSF), anti-AAV9 antibodies (serum, CSF), and T-cell reactivity to AAV9 and Construct I transgene product via enzyme-linked immunospot (ELISPOT; whole blood).

In some embodiments, disease progression may be assessed by administration of CLN2 CRS-MX to pediatric patients. In some embodiments, disease progression may be assessed by administration of CLN2 CRS-MX to adult patients. In some embodiments, provided herein is a method of treating ocular manifestations associated with CLN2 Batten disease in a subject in need thereof, said method comprising administering to the eye of said subject a recombinant adeno-associated virus (rAAV) comprising a capsid and a vector genome, wherein the rAAV is AAV9, and wherein the vector genome comprises:
an AAV 5′ inverted terminal repeat (ITR);
a promoter;
a CLN2 coding sequence encoding a human tripeptidyl peptidase 1 (TPP1) protein; and
an AAV 3′ ITR,
   wherein the method further comprises monitoring changes, or lack thereof, in said patient's CLN2 CRS-MX rating during and/or following administration of the vector. In some embodiments, the subject has a change from baseline in their CLN2 CRS-MX rating of +1 point, +2 points, +3 points, +4 points, +5 points, or +6 points. In some embodiments, the method slows or arrests progression of ocular manifestations associated with CLN2 Batten disease in a subject, determined by a slowed decrease in and/or maintenance of the subject's CLN2 CRS-MX rating over a period of 1 month or more, 2 months or more, 3 months or more, 6 months or more, 1 year or more, or 2 years or more.

In some embodiments, disease progression may be assessed by administration of CLN2 CRS-LX to pediatric patients. In some embodiments, provided herein is a method of treating ocular manifestations associated with CLN2 Batten disease in a subject in need thereof, said method comprising administering to the eye of said subject a recombinant adeno-associated virus (rAAV) comprising a capsid and a vector genome, wherein the rAAV is AAV9, and wherein the vector genome comprises:
an AAV 5′ inverted terminal repeat (ITR);
a promoter;
a CLN2 coding sequence encoding a human tripeptidyl peptidase 1 (TPP1) protein; and
an AAV 3′ ITR,
   wherein the method further comprises monitoring changes, or lack thereof, in said patient's CLN2 CRS-LX rating during and/or following administration of the vector. In some embodiments, the subject has a change from baseline in their CLN2 CRS-LX rating of +1 point, +2 points, +3 points, +4 points, +5 points, or +6 points. In some embodiments, the method slows or arrests progression of ocular manifestations associated with CLN2 Batten disease in a subject, determined by a slowed decrease in and/or maintenance of the subject's CLN2 CRS-LX rating over a period of 1 month or more, 2 months or more, 3 months or more, 6 months or more, 1 year or more, or 2 years or more.

4.4 Treatment System, Device, or Apparatus to be Used for a Treatment Method Described Herein

Also provided herein are treatment system, devices, and apparatuses to be used for a treatment method described herein, which may comprise one or more of the following: bottles, tubes, light source, microinjector, and foot pedal. In certain embodiments, the light source is a self-illuminating contact lens, which can be used to deposit vector in the back of the eye and in particular and to avoid damaging the optic disc, fovea and/or macula (see, e.g., Chalam et al., 2004, Ophthalmic surgery and lasers. 35. 76-77, which is incorporated by reference herein in its entirety). In certain embodiments, a self-illuminating contact lens is utilized during peripheral injection for visualizing the subretinal injection (see, e.g., Chalam et al., 2004, Ophthalmic surgery and lasers. 35. 76-77, which is incorporated by reference herein in its entirety). In certain embodiments, an optic fiber chandelier is utilized via a second trocar for visualizing the subretinal injection.

4.5 Combination Therapies

The methods provided herein may be combined with one or more additional therapies.

The additional therapies may be administered before, concurrently or subsequent to the gene therapy treatment. In some embodiments, the additional therapy is an enzyme replacement therapy (ERT) with recombinant TPP1 (e.g., Brineura® cerliponase alfa).

5. SEQUENCES

| SEQ | | |
|---|---|---|
| ID NO | Name | Sequence |
| 1 | human | MGLQACLLGLFALILSGKCSYSPEPDQRRTLPPGWVSLGRADPEEELSLTFALR |
| | Tripeptidyl- | QQNVERLSELVQAVSDPSSPQYGKYLTLENVADLVRPSPLTLHTVQKWLLAAGA |
| | peptidase 1 | QKCHSVITQDFLTCWLSIRQAELLLPGAEFHHYVGGPTETHVVRSPHPYQLPQA |
| | (UniProtKB/ | LAPHVDFVGGLHRFPPTSSLRQRPEPQVTGTVGLHLGVTPSVIRKRYNLTSQDV |
| | Swiss-Prot | GSGTSNNSQACAQFLEQYFHDSDLAQFMRLFGGNFAHQASVARVVGQQGRGRAG |
| | Accession | IEASLDVQYLMSAGANISTWVYSSPGRHEGQEPFLQWLMLLSNESALPHVHTVS |
| | O14773-1) | YGDDEDSLSSAYIQRVNTELMKAAARGLTLLFASGDSGAGCWSVSGRHQFRPTF |
| | | PASSPYVTTVGGTSFQEPFLITNEIVDYISGGGFSNVFPRPSYQEEAVTKFLSS |
| | | SPHLPPSSYFNASGRAYPDVAALSDGYWVVSNRVPIPWVSGTSASTPVFGGILS |
| | | LINEHRILSGRPPLGFLNPRLYQQHGAGLFDVTRGCHESCLDEEVEGQGFCSGP |
| | | GWDPVTGWGTPNFPALLKTLLNP |
| 2 | Homo sapiens | GGTGGTGGAATATAGAGCTCATGTGATCCGTCACATGACAGCAGATCCGCGGAA |
| | tripeptidyl | GGGCAGAATGGGACTCCAAGCCTGCCTCCTAGGGCTCTTTGCCCTCATCCTCTC |
| | peptidase 1 | TGGCAAATGCAGTTACAGCCCGGAGCCCGACCAGCGGAGGACGCTGCCCCCAGG |
| | (TPP1), mRNA | CTGGGTGTCCCTGGGCCGTGCGGACCCTGAGGAAGAGCTGAGTCTCACCTTTGC |
| | (NCBI | CCTGAGACAGCAGAATGTGGAAAGACTCTCGGAGCTGGTGCAGGCTGTGTCGGA |
| | Reference | TCCCAGCTCTCCTCAATACGGAAAATACCTGACCCTAGAGAATGTGGCTGATCT |
| | Sequence: | GGTGAGGCCATCCCCACTGACCCTCCACACGGTGCAAAAATGGCTCTTGGCAGC |
| | NM_000391.3) | CGGAGCCCAGAAGTGCCATTCTGTGATCACACAGGACTTTCTGACTTGCTGGCT |
| | | GAGCATCCGACAAGCAGAGCTGCTGCTCCCTGGGGCTGAGTTTCATCACTATGT |
| | | GGGAGGACCTACGGAAACCCATGTTGTAAGGTCCCCACATCCCTACCAGCTTCC |
| | | ACAGGCCTTGGCCCCCCATGTGGACTTTGTGGGGGGACTGCACCGTTTTCCCCC |
| | | AACATCATCCCTGAGGCAACGTCCTGAGCCGCAGGTGACAGGGACTGTAGGCCT |
| | | GCATCTGGGGGTAACCCCCTCTGTGATCCGTAAGCGATACAACTTGACCTCACA |
| | | AGACGTGGGCTCTGGCACCAGCAATAACAGCCAAGCCTGTGCCCAGTTCCTGGA |
| | | GCAGTATTTCCATGACTCAGACCTGGCTCAGTTCATGCGCCTCTTCGGTGGCAA |
| | | CTTTGCACATCAGGCATCAGTAGCCCGTGTGGTTGGACAACAGGGCCGGGGCCG |
| | | GGCCGGGATTGAGGCCAGTCTAGATGTGCAGTACCTGATGAGTGCTGGTGCCAA |
| | | CATCTCCACCTGGGTCTACAGTAGCCCTGGCCGGCATGAGGGACAGGAGCCCTT |
| | | CCTGCAGTGGCTCATGCTGCTCAGTAATGAGTCAGCCCTGCCACATGTGCATAC |
| | | TGTGAGCTATGGAGATGATGAGGACTCCCTCAGCAGCGCCTACATCCAGCGGGT |
| | | CAACACTGAGCTCATGAAGGCTGCCGCTCGGGGTCTCACCCTGCTCTTCGCCTC |
| | | AGGTGACAGTGGGGCCGGGTGTTGGTCTGTCTCTGGAAGACACCAGTTCCGCCC |
| | | TACCTTCCCTGCCTCCAGCCCCTATGTCACCACAGTGGGAGGCACATCCTTCCA |
| | | GGAACCTTTCCTCATCACAAATGAAATTGTTGACTATATCAGTGGTGGTGGCTT |
| | | CAGCAATGTGTTCCCACGGCCTTCATACCAGGAGGAAGCTGTAACGAAGTTCCT |
| | | GAGCTCTAGCCCCCACCTGCCACCATCCAGTTACTTCAATGCCAGTGGCCGTGC |
| | | CTACCCAGATGTGGCTGCACTTTCTGATGGCTACTGGGTGGTCAGCAACAGAGT |
| | | GCCCATTCCATGGGTGTCCGGAACCTCGGCCTCTACTCCAGTGTTTGGGGGGAT |
| | | CCTATCCTTGATCAATGAGCACAGGATCCTTAGTGGCCGCCCCCCTCTTGGCTT |
| | | TCTCAACCCAAGGCTCTACCAGCAGCATGGGGCAGGACTCTTTGATGTAACCCG |
| | | TGGCTGCCATGAGTCCTGTCTGGATGAAGAGGTAGAGGGCCAGGGTTTCTGCTC |
| | | TGGTCCTGGCTGGGATCCTGTAACAGGCTGGGGAACACCCAACTTCCCAGCTTT |
| | | GCTGAAGACTCTACTCAACCCCTGACCCTTTCCTATCAGGAGAGATGGCTTGTC |
| | | CCCTGCCCTGAAGCTGGCAGTTCAGTCCCTTATTCTGCCCTGTTGGAAGCCCTG |
| | | CTGAACCCTCAACTATTGACTGCTGCAGACAGCTTATCTCCCTAACCCTGAAAT |
| | | GCTGTGAGCTTGACTTGACTCCCAACCCTACCATGCTCCATCATACTCAGGTCT |
| | | CCCTACTCCTGCCTTAGATTCCTCAATAAGATGCTGTAACTAGCATTTTTTGAA |
| | | TGCCTCTCCCTCCGCATCTCATCTTTCTCTTTTCAATCAGGCTTTTCCAAAGGG |
| | | TTGTATACAGACTCTGTGCACTATTTCACTTGATATTCATTCCCCAATTCACTG |
| | | CAAGGAGACCTCTACTGTCACCGTTTACTCTTTCCTACCCTGACATCCAGAAAC |
| | | AATGGCCTCCAGTGCATACTTCTCAATCTTTGCTTTATGGCCTTTCCATCATAG |
| | | TTGCCCACTCCCTCTCCTTACTTAGCTTCCAGGTCTTAACTTCTCTGACTACTC |
| | | TTGTCTTCCTCTCTCATCAATTTCTGCTTCTTCATGGAATGCTGACCTTCATTG |
| | | CTCCATTTGTAGATTTTTGCTCTTCTCAGTTTACTCATTGTCCCCTGGAACAAA |
| | | TCACTGACATCTACAACCATTACCATCTCACTAAATAAGACTTTCTATCCAATA |
| | | ATGATTGATACCTCAAATGTAAGATGCGTGATACTCAACATTTCATCGTCCACC |
| | | TTCCCAACCCCAAACAATTCCATCTCGTTTCTTCTTGGTAAATGATGCTATGCT |
| | | TTTTCCAACCAAGCCAGAAACCTGTGTCATCTTTTCACCCCACCTTCAATCAAC |
| | | AAGTCCTCAATCAACAAGTCCTACTGACTGCACATCTTAAATATATCTTTATCA |
| | | GTCCACAAGTCCTTCCAATTATATTTCCCAAGTATATCTAGAACTTATCCACTT |
| | | ATATCCCCACTGCTACTACCTTAGTTTAGGGCTATATTCTCTTGAAAAAAAGTG |
| | | TCCTTACTTCCTGCCAATCCCCAAGTCATCTTCCAGAGTAAAATGCAAATCCCA |
| | | TCAGGCCACTTGGATGAAAACCCTTCAAGGATTACTGGATAGAATTCAGGCTTT |
| | | CCCCTCCAGCCCCCAATCATAGCTCACAAACCTTCCTTGCTATTTGTTCTTAAG |
| | | TAAAAAATCATTTTTCCTCCTCCCTCCCCAAACCCCAAGGAACTCTCACTCTTG |
| | | CTCAAGCTGTTCCGTCCCCTTACCACCCCTGATACAACTGCCAGGTTAATTTCC |
| | | AGAATTCTTGCAAGACTCAGTTCAGAAGTCACCTTCTTTCGTGAATGTTTTGAT |
| | | TCCCTGAGGCTACTTTATTTTGGTATGGCTGAAAAATCCTAGATTTTCTAAACA |
| | | AAACCTGTTTGAATCTTGGTTCTGATATGGACTAGGAGAGAGACTGGGTCAAGT |
| | | AAGCTTATCTCCCTGAGGCTGTTTCCTCGTCTGTTAAGTGTGAATATCAATACC |
| | | TGCCTTTCATAATCACCAGGGAATAAAGTGGAATAATGTTGATAACAGTGCTTG |
| | | GCACCTGGAAGTAGGTGGCAGATGTTAACGCCCTTCCTCCCTTGCACTGCGCCC |
| | | CCTGTGCCTACCTCTAGCATTGTAACGACCACGTAGTATTGAAATGGCCAGTTT |
| | | ACTTGTCTGCCTTCCTTTCCAAGACCGTTGGTGCCTAGAGGACTAGAATCGTGT |
| | | CCTATTTAACTTTGTGTTCCCAGGTCCTAGCTCAGGAGTTGGCAAATAAGAATT |
| | | AAATGTCTGCTACACCGAAAACCAAAAAAA |
| 3 | Human CLN2, | atgggactgc aggcctgtct gctgggactg ttcgccctga tcctgagcgg |
| | codon | caagtgcagc tacagccccg agcccgacca gagaagaaca ctgcctccag |
| | optimized | gctgggtgtc cctgggcaga gctgaccctg aagaggaact gagcctgacc |
| | | ttcgccctgc ggcagcagaa cgtggaaaga ctgagcgagc tggtgcaggc |
| | | cgtgtccgat cctagcagcc ctcagtacgg caagtacctg accctggaaa |
| | | acgtggccga cctcgtgcgg cctagccctc tgacactgca caccgtgcag |
| | | aagtggctgc tggctgccgg cgctcagaaa tgccactccg tgatcaccca |
| | | ggactttctg acctgttggc tgagcatccg gcaggccgaa ctgctgctgc |
| | | ctggggccga gtttcaccac tatgtgggcg gacccaccga gacacatgtc |
| | | gtgcgcagcc cacaccctta ccagctgcca caggctctgg cccctcacgt |
| | | ggactttgtg ggaggcctgc acagattccc cccaaccagc agcctgagac |
| | | agaggcctga gccacaagtg accggcacag tgggcctgca tctgggcgtg |
| | | acacctagcg tgatccggaa gcggtacaac ctgaccagcc aggatgtggg |
| | | cagcggcacc agcaacaata gccaggcctg cgcccagttc ctggaacagt |
| | | acttccacga cagcgatctg gcccagttca tgcggctgtt cggcggcaac |
| | | ttcgcacatc aggctagcgt ggccagagtc gtgggccagc agggaagagg |
| | | cagagccgga attgaggcct ccctggacgt gcagtacctg atgagcgctg |
| | | gcgccaacat cagcacctgg gtgtacagca gccccggcag acacgagggc |
| | | caggaacctt ttctgcagtg gctgatgctg ctgagcaacg agagcgccct |
| | | gcctcatgtg cacacagtgt cctacggcga cgacgaggac agcctgagca |
| | | gcgcctacat ccagagagtg aacaccgagc tgatgaaggc cgctgccagg |
| | | ggactgaccc tgctgtttgc ctctggcgat agcggagccg gctgttggag |
| | | tgtgtcaggc cggcaccagt tcagacccac ctttcctgcc agctccccct |
| | | acgtgacaac cgtgggcggc acctcctttc aggaaccctt cctgatcacc |
| | | aacgagatcg tggactacat cagcggcgga ggcttcagca acgtgttccc |
| | | cagacccagc taccaggaag aggccgtgac caagttcctg tcctccagcc |
| | | ctcatctgcc ccccagctcc tacttcaacg ccagcggcag agcctaccca |
| | | gatgtggccg ctctgtccga cggctactgg gtggtgtcca acagagtgcc |
| | | catcccttgg gtgtccggca caagcgccag cacccctgtg tttggcggca |
| | | tcctgtccct gatcaacgag cacagaatcc tgtccggcag accccccctg |
| | | ggcttcctga accctagact gtaccagcag cacggcgctg gcctgttcga |
| | | tgtgaccaga ggctgccacg agagctgcct ggacgaggaa gtggaaggcc |
| | | agggcttctg ttctggccct ggctgggatc ctgtgaccgg atggggcacc |
| | | cctaacttcc ccgccctgct gaaaacactg ctgaacccct gat |
| 4 | human | MRLFGGNFAHQASVARVVGQQGRGRAGIEASLDVQYLMSAGANISTWVYSSPGR |
| | Tripeptidyl- | HEGQEPFLQWLMLLSNESALPHVHTVSYGDDEDSLSSAYIQRVNTELMKAAARG |
| | peptidase 1 | LTLLFASGDSGAGCWSVSGRHQFRPTFPASSPYVTTVGGTSFQEPFLITNEIVD |
| | (UniProtKB/ | YISGGGFSNVFPRPSYQEEAVTKFLSSSPHLPPSSYFNASGRAYPDVAALSDGY |
| | Swiss-Prot | WVVSNRVPIPWVSGTSASTPVFGGILSLINEHRILSGRPPLGFLNPRLYQQHGA |
| | Accession | GLFDVTRGCHESCLDEEVEGQGFCSGPGWDPVTGWGTPNFPALLKTLLNP |
| | O14773-2) | |
| 5 | TPP1 | tcgaggacgg ggtgaactac gcctgaggat ccgatctttt tccctctgcc |
| | production | aaaaattatg gggacatcat gaagcccctt gagcatctga cttctggcta |
| | plasmid | ataaaggaaa tttattttca ttgcaatagt gtgttggaat tttttgtgtc |
| | | tctcactcgg aagcaattcg ttgatctgaa tttcgaccac ccataatacc |
| | | cattaccctg gtagataagt agcatggcgg gttaatcatt aactacaagg |
| | | aacccctagt gatggagttg gccactccct ctctgcgcgc tcgctcgctc |
| | | actgaggccg ggcgaccaaa ggtcgcccga cgcccgggct ttgcccgggc |
| | | ggcctcagtg agcgagcgag cgcgcagcct taattaacct aattcactgg |
| | | ccgtcgtttt acaacgtcgt gactgggaaa accctggcgt tacccaactt |
| | | aatcgccttg cagcacatcc ccctttcgcc agctggcgta atagcgaaga |
| | | ggcccgcacc gatcgccctt cccaacagtt gcgcagcctg aatggcgaat |
| | | gggacgcgcc ctgtagcggc gcattaagcg cggcgggtgt ggtggttacg |
| | | cgcagcgtga ccgctacact tgccagcgcc ctagcgcccg ctcctttcgc |
| | | tttcttccct tcctttctcg ccacgttcgc cggctttccc cgtcaagctc |
| | | taaatcgggg gctcccttta gggttccgat ttagtgcttt acggcacctc |
| | | gaccccaaaa aacttgatta gggtgatggt tcacgtagtg ggccatcgcc |
| | | ctgatagacg gtttttcgcc ctttgacgtt ggagtccacg ttctttaata |
| | | gtggactctt gttccaaact ggaacaacac tcaaccctat ctcggtctat |
| | | tottttgatt tataagggat tttgccgatt tcggcctatt ggttaaaaaa |
| | | tgagctgatt taacaaaaat ttaacgcgaa ttttaacaaa atattaacgc |
| | | ttacaattta ggtggcactt ttcggggaaa tgtgcgcgga acccctattt |
| | | gtttattttt ctaaatacat tcaaatatgt atccgctcat gagacaataa |
| | | ccctgataaa tgcttcaata atattgaaaa aggaagagta tgagtattca |
| | | acatttccgt gtcgccctta ttcccttttt tgcggcattt tgccttcctg |
| | | tttttgctca cccagaaacg ctggtgaaag taaaagatgc tgaagatcag |
| | | ttgggtgcac gagtgggtta catcgaactg gatctcaaca gcggtaagat |
| | | ccttgagagt tttcgccccg aagaacgttt tccaatgatg agcactttta |
| | | aagttctgct atgtggcgcg gtattatccc gtattgacgc cgggcaagag |
| | | caactcggtc gccgcataca ctattctcag aatgacttgg ttgagtactc |
| | | accagtcaca gaaaagcatc ttacggatgg catgacagta agagaattat |
| | | gcagtgctgc cataaccatg agtgataaca ctgcggccaa cttacttctg |
| | | acaacgatcg gaggaccgaa ggagctaacc gcttttttgc acaacatggg |
| | | ggatcatgta actcgccttg atcgttggga accggagctg aatgaagcca |
| | | taccaaacga cgagcgtgac accacgatgc ctgtagcaat ggcaacaacg |
| | | ttgcgcaaac tattaactgg cgaactactt actctagctt cccggcaaca |
| | | attaatagac tggatggagg cggataaagt tgcaggacca cttctgcgct |
| | | cggcccttcc ggctggctgg tttattgctg ataaatctgg agccggtgag |
| | | cgtgggtctc gcggtatcat tgcagcactg gggccagatg gtaagccctc |
| | | ccgtatcgta gttatctaca cgacggggag tcaggcaact atggatgaac |
| | | gaaatagaca gatcgctgag ataggtgcct cactgattaa gcattggtaa |
| | | ctgtcagacc aagtttactc atatatactt tagattgatt taaaacttca |
| | | tttttaattt aaaaggatct aggtgaagat cctttttgat aatctcatga |
| | | ccaaaatccc ttaacgtgag ttttcgttcc actgagcgtc agaccccgta |
| | | gaaaagatca aaggatcttc ttgagatcct ttttttctgc gcgtaatctg |
| | | ctgcttgcaa acaaaaaaac caccgctacc agcggtggtt ttttgccgg |
| | | atcaagagct accaactctt tttccgaagg taactggctt cagcagagcg |
| | | cagataccaa atactgttct tctagtgtag cogtagttag gccaccactt |
| | | caagaactct gtagcaccgc ctacatacct cgctctgcta atcctgttac |
| | | cagtggctgc tgccagtggc gataagtcgt gtcttaccgg gttggactca |
| | | agacgatagt taccggataa ggcgcagcgg tcgggctgaa cggggggttc |
| | | gtgcacacag cccagcttgg agcgaacgac ctacaccgaa ctgagatacc |
| | | tacagcgtga gctatgagaa agcgccacgc ttcccgaagg gagaaaggcg |
| | | gacaggtatc cggtaagcgg cagggtcgga acaggagagc gcacgaggga |
| | | gcttccaggg ggaaacgcct ggtatcttta tagtcctgtc gggtttcgcc |
| | | acctctgact tgagcgtcga tttttgtgat gctcgtcagg ggggcggagc |
| | | ctatggaaaa acgccagcaa cgcggccttt ttacggttcc tggccttttg |
| | | ctggcctttt gctcacatgt tctttcctgc gttatcccct gattctgtgg |
| | | ataaccgtat taccgccttt gagtgagctg ataccgctcg ccgcagccga |
| | | acgaccgagc gcagcgagtc agtgagcgag gaagcggaag agcgcccaat |
| | | acgcaaaccg cctctccccg cgcgttggcc gattcattaa tgcagctggc |
| | | acgacaggtt tcccgactgg aaagcgggca gtgagcgcaa cgcaattaat |
| | | gtgagttagc tcactcatta ggcaccccag gctttacact ttatgcttcc |
| | | ggctcgtatg ttgtgtggaa ttgtgagcgg ataacaattt cacacaggaa |
| | | acagctatga ccatgattac gccagattta attaaggcct taattaggct |
| | | gcgcgctcgc tcgctcactg aggccgcccg ggcaaagccc gggcgtcggg |
| | | cgacctttgg tcgcccggcc tcagtgagcg agcgagcgcg cagagaggga |
| | | gtggccaact ccatcactag gggttccttg tagttaatga ttaacccgcc |
| | | atgctactta tctaccaggg taatggggat cctctagaac tatagctagt |
| | | cgacattgat tattgactag ttattaatag taatcaatta cggggtcatt |
| | | agttcatagc ccatatatgg agttccgcgt tacataactt acggtaaatg |
| | | gcccgcctgg ctgaccgccc aacgaccccc gcccattgac gtcaataatg |
| | | acgtatgttc ccatagtaac gccaataggg actttccatt gacgtcaatg |
| | | ggtggactat ttacggtaaa ctgcccactt ggcagtacat caagtgtatc |
| | | atatgccaag tacgccccct attgacgtca atgacggtaa atggcccgcc |
| | | tggcattatg cccagtacat gaccttatgg gactttccta cttggcagta |
| | | catctacgta ttagtcatcg ctattaccat ggtcgaggtg agccccacgt |
| | | tctgcttcac tctccccatc tcccccccct ccccaccccc aattttgtat |
| | | ttatttattt tttaattatt ttgtgcagcg atgggggcgg gggggggggg |
| | | ggggcgcgcg ccaggcgggg cggggcgggg cgaggggcgg ggcggggcga |
| | | ggcggagagg tgcggcggca gccaatcaga goggcgcgct ccgaaagttt |
| | | ccttttatgg cgaggcggcg gcggcggcgg ccctataaaa agcgaagcgc |
| | | gcggcgggcg gggagtcgct gcgacgctgc cttcgccccg tgccccgctc |
| | | cgccgccgcc tcgcgccgcc cgccccggct ctgactgacc gcgttactcc |
| | | cacaggtgag cgggcgggac ggcccttctc ctccgggctg taattagcgc |
| | | ttggtttaat gacggcttgt ttcttttctg tggctgcgtg aaagccttga |
| | | ggggctccgg gagggccctt tgtgcggggg gagcggctcg gggggtgcgt |
| | | gcgtgtgtgt gtgcgtgggg agcgccgcgt gcggctccgc gctgcccggc |
| | | ggctgtgagc gctgcgggcg cggcgcgggg ctttgtgcgc tccgcagtgt |
| | | gcgcgagggg agcgcggccg ggggcggtgc cccgcggtgc ggggggggct |
| | | gcgaggggaa caaaggctgc gtgcggggtg tgtgcgtggg ggggtgagca |
| | | gggggtgtgg gcgcgtcggt cgggctgcaa ccccccctgc acccccctcc |
| | | ccgagttgct gagcacggcc cggcttcggg tgcggggctc cgtacggggc |
| | | gtggcgcggg gctcgccgtg ccgggcgggg ggtggcggca ggtgggggtg |
| | | ccgggcgggg cggggccgcc tcgggccggg gagggctcgg gggaggggcg |
| | | cggcggcccc cggagcgccg gcggctgtcg aggcgcggcg agccgcagcc |
| | | attgcctttt atggtaatcg tgcgagaggg cgcagggact tcctttgtcc |
| | | caaatctgtg cggagccgaa atctgggagg cgccgccgca ccccctctag |
| | | cgggcgcggg gcgaagcggt gcggcgccgg caggaaggaa atgggcgggg |
| | | agggccttcg tgcgtcgccg cgccgccgtc cccttctccc tctccagcct |
| | | cggggctgtc cgcgggggga cggctgcctt cgggggggac ggggcagggc |
| | | ggggttcggc ttctggcgtg tgaccggcgg ctctagagcc tctgctaacc |
| | | atgttcatgc cttcttcttt ttcctacagc tcctgggcaa cgtgctggtt |
| | | attgtgctgt ctcatcattt tggcaaagaa ttcacgcgtg ccaccatggg |
| | | actgcaggcc tgtctgctgg gactgttcgc cctgatcctg agcggcaagt |
| | | gcagctacag ccccgagccc gaccagagaa gaacactgcc tccaggctgg |
| | | gtgtccctgg gcagagctga ccctgaagag gaactgagcc tgaccttcgc |
| | | cctgcggcag cagaacgtgg aaagactgag cgagctggtg caggccgtgt |
| | | ccgatcctag cagccctcag tacggcaagt acctgaccct ggaaaacgtg |
| | | gccgacctcg tgcggcctag ccctctgaca ctgcacaccg tgcagaagtg |
| | | gctgctggct gccggcgctc agaaatgcca ctccgtgatc acccaggact |
| | | ttctgacctg ttggctgagc atccggcagg ccgaactgct gctgcctggg |
| | | gccgagtttc accactatgt gggcggaccc accgagacac atgtcgtgcg |
| | | cagcccacac ccttaccagc tgccacaggc tctggcccct |
| | | cacgtggact ttgtgggagg cctgcacaga ttccccccaa ccagcagcct |
| | | gagacagagg cctgagccac aagtgaccgg cacagtgggc ctgcatctgg |
| | | gcgtgacacc tagcgtgatc cggaagcggt acaacctgac cagccaggat |
| | | gtgggcagcg gcaccagcaa caatagccag gcctgcgccc agttcctgga |
| | | acagtacttc cacgacagcg atctggccca gttcatgcgg ctgttcggcg |
| | | gcaacttcgc acatcaggct agcgtggcca gagtcgtggg ccagcaggga |
| | | agaggcagag ccggaattga ggcctccctg gacgtgcagt acctgatgag |
| | | cgctggcgcc aacatcagca cctgggtgta cagcagcccc ggcagacacg |
| | | agggccagga accttttctg cagtggctga tgctgctgag caacgagagc |
| | | gccctgcctc atgtgcacac agtgtcctac ggcgacgacg aggacagcct |
| | | gagcagcgcc tacatccaga gagtgaacac cgagctgatg aaggccgctg |
| | | ccaggggact gaccctgctg tttgcctctg gcgatagcgg agccggctgt |
| | | tggagtgtgt caggccggca ccagttcaga cccacctttc ctgccagctc |
| | | cccctacgtg acaaccgtgg gcggcacctc ctttcaggaa cccttcctga |
| | | tcaccaacga gatcgtggac tacatcagcg goggaggctt cagcaacgtg |
| | | ttccccagac ccagctacca ggaagaggcc gtgaccaagt tcctgtcctc |
| | | cagccctcat ctgcccccca gctcctactt caacgccagc ggcagagcct |
| | | acccagatgt ggccgctctg tccgacggct actgggtggt gtccaacaga |
| | | gtgcccatcc cttgggtgtc cggcacaagc gccagcaccc ctgtgtttgg |
| | | cggcatcctg tccctgatca acgagcacag aatcctgtcc ggcagacccc |
| | | ccctgggctt cctgaaccct agactgtacc agcagcacgg cgctggcctg |
| | | ttcgatgtga ccagaggctg ccacgagagc tgcctggacg aggaagtgga |
| | | aggccagggc ttctgttctg gccctggctg ggatcctgtg accggatggg |
| | | gcacccctaa cttccccgcc ctgctgaaaa cactgctgaa cccctgatga |
| | | c |
| 6 | AAV9 capsid | MAADGYLPDWLEDNLSEGIREWWALKPGAPQPKANQQHQDNARGLVLPGYKYLG |
| | | PGNGLDKGEPVNAADAAALEHDKAYDQQLKAGDNPYLKYNHADAEFQERLKEDT |
| | | SFGGNLGRAVFQAKKRLLEPLGLVEEAAKTAPGKKRPVEQSPQEPDSSAGIGKS |
| | | GAQPAKKRLNFGQTGDTESVPDPQPIGEPPAAPSGVGSLTMASGGGAPVADNNE |
| | | GADGVGSSSGNWHCDSQWLGDRVITTSTRTWALPTYNNHLYKQISNSTSGGSSN |
| | | DNAYFGYSTPWGYFDFNRFHCHFSPRDWQRLINNNWGFRPKRLNFKLFNIQVKE |
| | | VTDNNGVKTIANNLTSTVQVFTDSDYQLPYVLGSAHEGCLPPFPADVFMIPQYG |
| | | YLTLNDGSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSAQAQAQTG |
| | | WVQNQGILPGMVWQDRDVYLQGPIWAKIPHTDGNFHPSPLMGGFGMKHPPPQIL |
| | | IKNTPVPADPPTAFNKDKLNSFITQYSTGQVSVEIEWELQKENSKRWNPEIQYT |
| | | SNYYKSNNVEFAVNTEGVYSEPRPIGTRYLTRNL |
| 7 | AAV9 capsid | atggctgccg atggttatct tccagattgg ctcgaggaca accttagtga |
| | | aggaattcgc gagtggtggg ctttgaaacc tggagcccct caacccaagg |
| | | caaatcaaca acatcaagac aacgctcgag gtcttgtgct tccgggttac |
| | | aaataccttg gacccggcaa cggactcgac aagggggagc cggtcaacgc |
| | | agcagacgcg goggccctcg agcacgacaa ggcctacgac cagcagctca |
| | | aggccggaga caacccgtac ctcaagtaca accacgccga cgccgagttc |
| | | caggagcggc tcaaagaaga tacgtctttt gggggcaacc tcgggcgagc |
| | | agtcttccag gccaaaaaga ggcttcttga acctcttggt ctggttgagg |
| | | aagcggctaa gacggctcct ggaaagaaga ggcctgtaga gcagtctcct |
| | | caggaaccgg actcctccgc gggtattggc aaatcgggtg cacagcccgc |
| | | taaaaagaga ctcaatttcg gtcagactgg cgacacagag tcagtcccag |
| | | accctcaacc aatcggagaa cctcccgcag ccccctcagg tgtgggatct |
| | | cttacaatgg cttcaggtgg tggcgcacca gtggcagaca ataacgaagg |
| | | tgccgatgga gtgggtagtt cctcgggaaa ttggcattgc gattcccaat |
| | | ggctggggga cagagtcatc accaccagca cccgaacctg ggccctgccc |
| | | acctacaaca atcacctcta caagcaaatc tocaacagca catctggagg |
| | | atcttcaaat gacaacgcct acttcggcta cagcaccccc tgggggtatt |
| | | ttgacttcaa cagattccac toccacttct caccacgtga ctggcagcga |
| | | ctcatcaaca acaactgggg attccggcct aagcgactca acttcaagct |
| | | cttcaacatt caggtcaaag aggttacgga caacaatgga gtcaagacca |
| | | togccaataa ccttaccagc acggtccagg tcttcacgga ctcagactat |
| | | cagctcccgt acgtgctcgg gtcggctcac gagggctgcc tcccgccgtt |
| | | cccagcggac gttttcatga ttcctcagta cgggtatctg acgcttaatg |
| | | atggaagcca ggccgtgggt cgttcgtcct tttactgcct ggaatatttc |
| | | ccgtcgcaaa toctaagaac gggtaacaac ttccagttca gctacgagtt |
| | | tgagaacgta cctttccata gcagctacgc tcacagccaa agcctggacc |
| | | gactaatgaa tccactcatc gaccaatact tgtactatct ctcaaagact |
| | | attaacggtt ctggacagaa tcaacaaacg ctaaaattca gtgtggccgg |
| | | acccagcaac atggctgtcc agggaagaaa ctacatacct ggacccagct |
| | | accgacaaca acgtgtctca accactgtga ctcaaaacaa caacagcgaa |
| | | tttgcttggc ctggagcttc ttcttgggct ctcaatggac gtaatagctt |
| | | gatgaatcct ggacctgcta tggccagcca caaagaagga gaggaccgtt |
| | | totttccttt gtctggatct ttaatttttg gcaaacaagg aactggaaga |
| | | gacaacgtgg atgcggacaa agtcatgata accaacgaag aagaaattaa |
| | | aactactaac coggtagcaa cggagtccta tggacaagtg gccacaaacc |
| | | accagagtgc ccaagcacag gogcagaccg gctgggttca aaaccaagga |
| | | atacttccgg gtatggtttg gcaggacaga gatgtgtacc tgcaaggacc |
| | | catttgggcc aaaattcctc acacggacgg caactttcac ccttctccgc |
| | | tgatgggagg gtttggaatg aagcacccgc ctcctcagat cctcatcaaa |
| | | aacacacctg tacctgcgga tcctccaacg gccttcaaca aggacaagct |
| | | gaactctttc atcacccagt attctactgg ccaagtcagc gtggagatcg |
| | | agtgggagct gcagaaggaa aacagcaagc gotggaaccc ggagatccag |
| | | tacacttcca actattacaa gtctaataat gttgaatttg ctgttaatac |
| | | tgaaggtgta tatagtgaac cccgccccat tggcaccaga tacctgactc |
| | | gtaatctgta a |
| 8 | Constructed | ctgcgcgctc gctcgctcac tgaggccgcc cgggcaaagc ccgggcgtcg |
| | Sequence | ggcgaccttt ggtcgcccgg cctcagtgag cgagcgagcg cgcagagagg |
| | | gagtggccaa ctccatcact aggggttcct tgtagttaat gattaacccg |
| | | ccatgctact tatctaccag ggtaatgggg atcctctaga actatagcta |
| | | gtcgacattg attattgact agttattaat agtaatcaat tacggggtca |
| | | ttagttcata gcccatatat ggagttccgc gttacataac ttacggtaaa |
| | | tggcccgcct ggctgaccgc ccaacgaccc ccgcccattg acgtcaataa |
| | | tgacgtatgt tcccatagta acgccaatag ggactttcca ttgacgtcaa |
| | | tgggtggact atttacggta aactgcccac ttggcagtac atcaagtgta |
| | | tcatatgcca agtacgcccc ctattgacgt caatgacggt aaatggcccg |
| | | cctggcatta tgcccagtac atgaccttat gggactttcc tacttggcag |
| | | tacatctacg tattagtcat cgctattacc atggtcgagg tgagccccac |
| | | gttctgcttc actctcccca tctccccccc ctccccaccc ccaattttgt |
| | | atttatttat tttttaatta ttttgtgcag cgatgggggc gggggggggg |
| | | ggggggcgcg cgccaggcgg ggcggggcgg ggcgaggggc ggggcggggc |
| | | gaggcggaga ggtgcggcgg cagccaatca gagcggcgcg ctccgaaagt |
| | | ttccttttat ggcgaggcgg cggcggcggc ggccctataa aaagcgaagc |
| | | gcgcggcggg cggggagtcg ctgcgacgct gccttcgccc cgtgccccgc |
| | | tccgccgccg cctcgcgccg cccgccccgg ctctgactga ccgcgttact |
| | | cccacaggtg agcgggcggg acggcccttc tcctccgggc tgtaattagc |
| | | gcttggttta atgacggctt gtttcttttc tgtggctgcg tgaaagcctt |
| | | gaggggctcc gggagggccc tttgtgcggg gggagcggct cggggggtgc |
| | | gtgcgtgtgt gtgtgcgtgg ggagcgccgc gtgcggctcc gcgctgcccg |
| | | gcggctgtga gcgctgcggg cgcggcgcgg ggctttgtgc gctccgcagt |
| | | gtgcgcgagg ggagcgcggc cgggggcggt gccccgcggt gcgggggggg |
| | | ctgcgagggg aacaaaggct gcgtgcgggg tgtgtgcgtg ggggggtgag |
| | | cagggggtgt gggcgcgtcg gtcgggctgc aaccccccct gcacccccct |
| | | ccccgagttg ctgagcacgg cccggcttcg ggtgcggggc tccgtacggg |
| | | gcgtggcgcg gggctcgccg tgccgggcgg ggggtggcgg caggtggggg |
| | | tgccgggcgg ggcggggccg cctcgggccg gggagggctc gggggagggg |
| | | cgcggcggcc cccggagcgc cggcggctgt cgaggcgcgg cgagccgcag |
| | | ccattgcctt ttatggtaat cgtgcgagag ggcgcaggga cttcctttgt |
| | | cccaaatctg tgcggagccg aaatctggga ggcgccgccg caccccctct |
| | | agcgggcgcg gggcgaagcg gtgcggcgcc ggcaggaagg aaatgggcgg |
| | | ggagggcctt cgtgcgtcgc cgcgccgccg tccccttctc cctctccagc |
| | | ctcggggctg tccgcggggg gacggctgcc ttcggggggg acggggcagg |
| | | gcggggttcg gcttctggcg tgtgaccggc ggctctagag cctctgctaa |
| | | ccatgttcat gccttcttct ttttcctaca gctcctgggc aacgtgctgg |
| | | ttattgtgct gtctcatcat tttggcaaag aattcacgcg tgccaccatg |
| | | ggactgcagg cctgtctgct gggactgttc gccctgatcc tgagcggcaa |
| | | gtgcagctac agccccgagc ccgaccagag aagaacactg cctccaggct |
| | | gggtgtccct gggcagagct gaccctgaag aggaactgag cctgaccttc |
| | | gccctgcggc agcagaacgt ggaaagactg agcgagctgg tgcaggccgt |
| | | gtccgatcct agcagccctc agtacggcaa gtacctgacc ctggaaaacg |
| | | tggccgacct cgtgcggcct agccctctga cactgcacac cgtgcagaag |
| | | tggctgctgg ctgccggcgc tcagaaatgc cactccgtga tcacccagga |
| | | ctttctgacc tgttggctga gcatccggca ggccgaactg ctgctgcctg |
| | | gggccgagtt tcaccactat gtgggcggac ccaccgagac acatgtcgtg |
| | | cgcagcccac acccttacca gctgccacag gctctggccc ctcacgtgga |
| | | ctttgtggga ggcctgcaca gattcccccc aaccagcagc ctgagacaga |
| | | ggcctgagcc acaagtgacc ggcacagtgg gcctgcatct gggcgtgaca |
| | | cctagcgtga tccggaagcg gtacaacctg accagccagg atgtgggcag |
| | | cggcaccagc aacaatagcc aggcctgcgc ccagttcctg gaacagtact |
| | | tccacgacag cgatctggcc cagttcatgc ggctgttcgg cggcaacttc |
| | | gcacatcagg ctagcgtggc cagagtcgtg ggccagcagg gaagaggcag |
| | | agccggaatt gaggcctccc tggacgtgca gtacctgatg agcgctggcg |
| | | ccaacatcag cacctgggtg tacagcagcc ccggcagaca cgagggccag |
| | | gaaccttttc tgcagtggct gatgctgctg agcaacgaga gcgccctgcc |
| | | tcatgtgcac acagtgtcct acggcgacga cgaggacagc ctgagcagcg |
| | | cctacatcca gagagtgaac accgagctga tgaaggccgc tgccagggga |
| | | ctgaccctgc tgtttgcctc tggcgatagc ggagccggct gttggagtgt |
| | | gtcaggccgg caccagttca gacccacctt tcctgccagc tccccctacg |
| | | tgacaaccgt gggcggcacc tcctttcagg aacccttcct gatcaccaac |
| | | gagatcgtgg actacatcag cggcggaggc ttcagcaacg tgttccccag |
| | | acccagctac caggaagagg ccgtgaccaa gttcctgtcc tccagccctc |
| | | atctgccccc cagctcctac ttcaacgcca goggcagagc ctacccagat |
| | | gtggccgctc tgtccgacgg ctactgggtg gtgtccaaca gagtgcccat |
| | | cccttgggtg tccggcacaa gcgccagcac ccctgtgttt ggcggcatcc |
| | | tgtccctgat caacgagcac agaatcctgt ccggcagacc ccccctgggc |
| | | ttcctgaacc ctagactgta ccagcagcac ggcgctggcc tgttcgatgt |
| | | gaccagaggc tgccacgaga gctgcctgga cgaggaagtg gaaggccagg |
| | | gcttctgttc tggccctggc tgggatcctg tgaccggatg gggcacccct |
| | | aacttccccg ccctgctgaa aacactgctg aacccctgat gactcgagga |
| | | cggggtgaac tacgcctgag gatccgatct ttttccctct gccaaaaatt |
| | | atggggacat catgaagccc cttgagcatc tgacttctgg ctaataaagg |
| | | aaatttattt tcattgcaat agtgtgttgg aattttttgt gtctctcact |
| | | cggaagcaat tcgttgatct gaatttcgac cacccataat acccattacc |
| | | ctggtagata agtagcatgg cgggttaatc attaactaca aggaacccct |
| | | agtgatggag ttggccactc cctctctgcg cgctcgctcg ctcactgagg |
| | | ccgggcgacc aaaggtcgcc cgacgcccgg gctttgcccg ggcggcctca |
| | | gtgagcgagc gagcgcgcag |
| 9 | AAV9 | MAADGYLPDWLEDNLSEGIREWWALKPGAPQPKANQQHQDNARGLVLPGYKYLG |
| | | PGNGLDKGEPVNAADAAALEHDKAYDQQLKAGDNPYLKYNHADAEFQERLKEDT |
| | | SFGGNLGRAVFQAKKRLLEPLGLVEEAAKTAPGKKRPVEQSPQEPDSSAGIGKS |
| | | GAQPAKKRLNFGQTGDTESVPDPQPIGEPPAAPSGVGSLTMASGGGAPVADNNE |
| | | GADGVGSSSGNWHCDSQWLGDRVITTSTRTWALPTYNNHLYKQISNSTSGGSSN |
| | | DNAYFGYSTPWGYFDFNRFHCHFSPRDWQRLINNNWGFRPKRLNFKLFNIQVKE |
| | | VTDNNGVKTIANNLTSTVQVFTDSDYQLPYVLGSAHEGCLPPFPADVFMIPQYG |
| | | YLTLNDGSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSAQAQAQTG |
| | | WVQNQGILPGMVWQDRDVYLQGPIWAKIPHTDGNFHPSPLMGGFGMKHPPPQIL |
| | | IKNTPVPADPPTAFNKDKLNSFITQYSTGQVSVEIEWELQKENSKRWNPEIQYT |
| | | SNYYKSNNVEFAVNTEGVYSEPRPIGTRYLTRNL |

### 6. EXAMPLES

The examples in this section (i.e., section 6) are offered by way of illustration, and not by way of limitation.

### 6.1 Example 1: Protocol for Treating Human Subjects and Assessing Efficacy of Treatment with Construct I

This Example provides an exemplary protocol for the treatment of human subjects to allow an assessment of the efficacy of Construct I treatment. Construct I may be administered at a dose of about or at least about 2×10¹⁰ GC/eye or about or at least about 6×10¹⁰ GC/eye. Construct I may be administered subretinally in a volume of 200 μL at a concentration of about 1×10¹¹ GC/mL to about 3.0×10¹¹ GC/mL.

6.1.1 Patient Population

Patients treated in accordance with the methods described in this example carry biallelic CLN2 mutations and have decreased leukocyte TPP1 activity. Patients also exhibit clinical signs or symptoms consistent with CLN2 disease (e.g., developmental delay, developmental decline, seizure, vision loss, or other signs symptoms) or have an older sibling with confirmed CLN2 diagnosis.

Patients are receiving intracerebroventricular Brineura enzyme replacement therapy. Patients further have CRT at baseline ≤210 μm and CRT at baseline ≥140 μm in both eyes and are at most 84 months old. Alternatively, Patients may be presymptomatic and have CRT at baseline >210 μm in both eyes and are between 12 and 48 months old. If CLN2 disease was diagnosed in an asymptomatic patient due to family history of CLN2 disease in a sibling, then the onset of vision loss in the affected sibling must have occurred at age 84 months or less. Patients may also have a CRT at baseline <140 μm in both eyes and Weill Cornell Ophthalmic Severity Score <5 (with no age limit).

Patients have visual acuity (VA) measurable with OKN-based VA test.

Patients may receive prednisolone (1 mg/kg/day for 7 days (i.e., Day −3 through Day 4) with a maximum dose of 40 mg/day) concurrently with Construct I treatment. After 7 days (3 days preoperative, day of surgery, 3 days postoperative), patients may receive a decreased dose of 0.5 mg/kg/day with a maximum dose of 20 mg/day for an additional 10 days (i.e., Day 5 through Day 14).

Patients may be receiving intracerebroventricular Brineura enzyme replacement therapy every 2 weeks for treatment of CLN2 Batten disease.

6.1.2 Efficacy Endpoints

Primary end-points for efficacy is a change from baseline in CRT as measured by SD-OCT at Day 360. Other efficacy endpoints include:
AAV9 detected by PCR in serum and urine,
Change from baseline in VA as measured by OKN at Day 360 (or longer),
Change from baseline in ONL thickness in the central 1 mm as measured by SD-OCT at Day 360 (or longer),
Change from baseline in measures and appearance of retinal layers over time as measured by SD-OCT at Day 360,
Change from baseline in pupillary light reflex as measured by pupillometry over time at Day 360,
Change from baseline in macular thickness/volume as measured by SD-OCT at Day 360,
Time to accelerated decline phase of retinal degeneration, defined as reaching 210 μm CRT, Time from CRT≤210 μm to 30 μm loss,
Change from baseline in CRT as measured by SD-OCT at Day 360,
Change from baseline in SD-OCT anatomical markers, e.g., parafoveal ellipsoid zone, ELM, RNFL,
Change from baseline in fundus appearance on fundus photography over time as measured by the modified WCBS,
Change from baseline in FAF at Day 360, Time to late stage disease, defined as CRT ≤110 μm,
Change from baseline in caregiver-reported visual outcome over time as assessed using PedEyeQ,
Change from baseline in adaptive behaviors based on cognitive ability over time as assessed using the VABs III and MSEL-Visual Reception,
Change from baseline in the Hamburg Scale Motor Function score using the treated eye and control eye,
Change from baseline in the modified Hamburg Scale Motor Function score using the treated eye and control eye.

6.1.3 Pharmacodynamics Endpoints

Levels of TPP1 in aqueous humor, serum and CSF are also evaluated. AAV9 concentrations are measured in the CSF. Antibodies to AAV9 may be measured in serum and CSF, and antibodies to the transgene product of Construct I (TPP1) may be measured in serum, CSF and aqueous humor. T cell reactivity to AAV9 and Construct I TP is tested in PBMCs using ELISPOT with whole blood.

6.1.4 Efficacy Assessments

Efficacy of treatment with Construct I may be evaluated using various assays. For example, retinal imaging may be conducted using Spectral Domain-Optical Coherence Tomography (SD-OCT) (bilateral macula and optic disc) using the Heidelberg Spectralis module imaging system.

Axial length of the treated and control eye may be performed using A-scan ultrasonography. Visual Acuity with Optokinetic Nystagmus Testing (OKN testing) may be conducted.

Fundus autofluorescence of the study eye may be performed using the Heidelberg Spectralis OCT instrument with Flex Module.

Multicolor fundus imaging of the study eye may be performed using a Heidelberg Spectralis OCT instrument with Flex Module

Pupillary light reflex with pupillometry may be conducted.

PedEyeQ may be used to evaluate the effect of childhood eye conditions on the child's eye-related quality of life and functional vision for children up to 17 years of age (Hatt et al, 2019, Am J Ophthalmol. 200:201-17). The PedEyeQ has multiple versions for children, proxy, and parent and can be tailored to the age groups of 0 to 4 years and 5 to 11 years. Each proxy questionnaire by age group consists of 3 common domains (Functional Vision, Bothered by Eyes/Vision, Social) and 2 domains (Frustration/Worry and Eyecare) intended only for patients aged 5 years and above. Each domain consists of 5 to 10 items. Caregivers can select a response for each item of “never,” “sometimes,” or “all the time” that corresponds to a score of 2, 1, or 0, respectively. Each domain can be scored independently by the average Rasch-calibrated value of all completed items for the domain. Domain scores can be converted to a 0 to 100 scale, where 0 is considered the worst possible and 100 is considered the best possible measure of quality of life or functional vision.

The Vineland Adaptive Behavior Scales (VABS)-III (Sparrow et al, 2016, Vineland Adaptive Behavior Scales. 3rd ed. Bloomington, MN: Pearson) may be used to assess adaptive behavior based on neurocognition in individuals from infancy to age 90 years. The 4 domains of Communication, Daily Living Skills, Socialization, and Motor Skills can be assessed, as these are appropriate for children aged <7 years. Items in each of the domains are scored from 2 to 0, based on the frequency that the individual demonstrates each adaptive skill/behavior, with 2 corresponding to almost always and 0 to never. The individual items are tallied and then calculated as a composite score to be compared against a standard, age matched bell curve with a mean of 100 and a standard deviation of 15. Depending on what percentile the child's score reaches, their overall adaptive behavior ability can be recorded as low, moderately low, adequate, moderately high, or high.

The Mullen Scales of Early Learning (MSEL) is a standardized assessment that is commonly used as a measure of cognitive development (Mullen, 1995, Scales of Early Learning (AGS ed.). Circle Pines, MN: American Guidance Service Inc.). The MSEL is organized into 5 subscales measuring (a) gross motor, (b) fine motor, (c) visual reception (or non-verbal problem solving), (d) receptive language, and (e) expressive language skills. Each subscale can be standardized to calculate a standard score, percentile, and age-equivalent score.

MSEL has been adapted for use in this study as a clinical outcome assessment to measure a potential effect of treatment on visual processing and problem solving (i.e., the MSEL-Visual Reception). In addition to the standard visual reception subscale, a modified version (first 7 items) of the visual reception domain may be assessed, i.e., with emphasis on visual fixation and tracking while minimizing reliance on cognition and fine motor dexterity. Examples include fixating on and tracking a triangle, tracking a moving bull's eye 180 degrees, and looking for a dropped spoon.

A CLN2 Clinical Rating Scale (CRS) has been developed specific to CLN2 disease to assess individuals' change in Motor Function, Language, Seizure, and Vision over time. The original 12-point Hamburg scale (Steinfeld et al, 2002, Am J Med Genet. 112(4):347-54) was developed to assess patients both retrospectively, as well as prospectively. It has been used in the largest longitudinal natural history data collection (Nickel et al, 2018, Lancet Child Adolesc Health. 2(8):582-590. doi: 10.1016/S2352-4642(18) 30179-2. Epub 2018 Jul. 2). In 2018, an update to the motor and language domain of the scale was published (Wyrwich et al, 2018, J Inborn Errors Metab Screen. 6:1-7) whereby the scale wording for motor and language scores gives a more detailed definition in order to be used in prospective data collection in clinical trials. The motor and language domains form a combined 0- to 6-point score. The highest score for each domain is 3 points, corresponding to normal or maximum ability, whereas a score of 0 indicates no ability left in that domain. These scales have been previously used in clinical studies, such as the 6-point Motor-Language scale used to assess Brineura's clinical efficacy (Schulz et al, 2018, N Engl J Med. 378(20):1898-1907). The Motor Function scale can be assessed using both the original Hamburg scale and 2018 modified version. The patient can perform each test twice—once with each eye occluded so that an OD (right eye) score and OS (left eye) score are recorded. In total, 4 scores can be recorded for the 2 scales.

6.1.5 Safety Assessments

The safety of administration of Construct I may be assessed by, for example, ophthalmic exams, physical exams vital sign assessment or clinical safety laboratory assessments (including, e.g., serum chemistry, hematologic measurements and urinalysis).

6.1.6 Vector Shedding

Vector transgenes have the potential to spread to unintended recipients from shedding (release of vectors that did not infect the target cells and were cleared from the body via feces or bodily fluids), mobilization (transgene replication and transfer out of the target cell), or germ line transmission (genetic transmission to offspring through semen). Vector shedding may be determined for example by measuring vector DNA in biological fluids such as tears, serum or urine using quantitative polymerase chain reaction.

Sampling of blood (serum), urine, and tears may be performed for Construct I patients for measurement of AAV9 vector concentrations.

Shedding data collected in these biological fluids can provide a shedding profile of Construct I in the target patient population and may be used to estimate the potential of transmission to untreated individuals. Shedding may be measured using quantitative polymerase chain reaction (PCR).

### 6.2 Example 2: Subretinal Injection of Construct I to Cynomolgus Monkeys Leads to Supraphysiological Levels of TPP1 in the Eye

In late infantile neuronal ceroid lipofuscinosis (CLN2) disease, mutations in the CLN2 gene lead to deficient TPP1 enzymatic activity, resulting in profound neurodegeneration within the central nervous system and retina. There is no currently approved therapy to treat the ocular manifestations of CLN2 disease and visual loss presents a critical unmet need. The aim of this study was to administer Construct I by subretinal injection and produce sufficient levels of TPP1 enzyme to prevent the ocular manifestations of CLN2 disease. Because murine models of CLN2 disease do not demonstrate overt retinal pathology the pharmacodynamics, biodistribution, immunogenicity and toxicity of Construct I was assessed in cynomolgus monkeys. Groups of cynomolgus monkeys (4-5/group) were administered Construct I by subretinal injection (100 μL) at doses ≥1×10¹⁰ GC/eye and transgene expression (TPP1 concentration) in the serum, aqueous and vitreous humor, and biodistribution were assessed for up to 13 weeks. Increases in TPP1 concentration were seen in the aqueous and vitreous humor, but not serum. Transgene expression was sustained throughout 3 months, however, TPP1 concentrations partly decreased in some eyes with development of serum anti-TPP1 antibodies. In the eye, high levels of vector DNA were observed in retina and choroid/RPE, both at the area of the bleb (temporal) and non-bleb (superior, inferior and nasal quadrants). Vector DNA was also present at low levels in the optic nerve, optic chiasm and occipital lobe, but not other brain regions, suggesting anterograde tropism. Immunostaining, showed that TPP1 protein was present in widespread areas of the retina, predominantly within photoreceptors, but also in other cell layers to a lesser degree. At 1×10¹⁰ GC/eye, TPP1 concentrations were 150-(aqueous) and 200-fold (vitreous) greater than untreated animals. This demonstrates that subretinal injection of Construct I may provide sufficient levels of TPP1 to attenuate the retinal degeneration seen in CLN2 patients.

### 6.3 Example 3: Subretinal Injection of Construct I to Cynomolgus Monkeys Leads to Supraphysiological Levels of TPP1 in the Eye

The purpose of this study was to evaluate levels of human TPP1 in the retinas of cynomolgus monkeys after subretinal injection of Construct I, a recombinant adeno-associated virus of serotype 9 (AAV9) containing a human CLN2 (hCLN2) expression cassette.

6.3.1 Methods

Groups of young cynomolgus monkeys (n=5/group; −2-3 years old) were administered a single subretinal injection (100 μL) of Construct I into both eyes at doses of 0 (vehicle) or >1×10¹⁰ GC/eye. Animals were euthanized either 4 (2/group) or 13 (3/group) weeks after dosing. In this study an assessment of TPP1 concentration in aqueous humor, vitreous humor (terminal sample) and serum, immunogenicity (anti-AAV9 neutralizing antibodies [NAbs] and anti-TPP1 antibodies) in the serum, biodistribution (vector DNA) and TPP1 immunostaining were included.

TPP1 concentration was determined by an electrochemiluminescent (ECL) immunoassay implemented using the Meso Scale Discovery (MSD) platform.

6.3.2 Results

Some Construct I-treated animals were positive for NAbs prior to dosing which did not have any notable effect on pharmacodynamics or biodistribution after administration of Construct I.

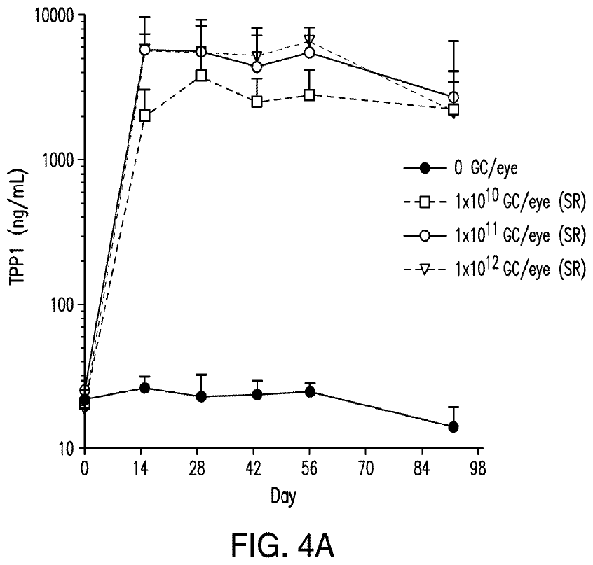

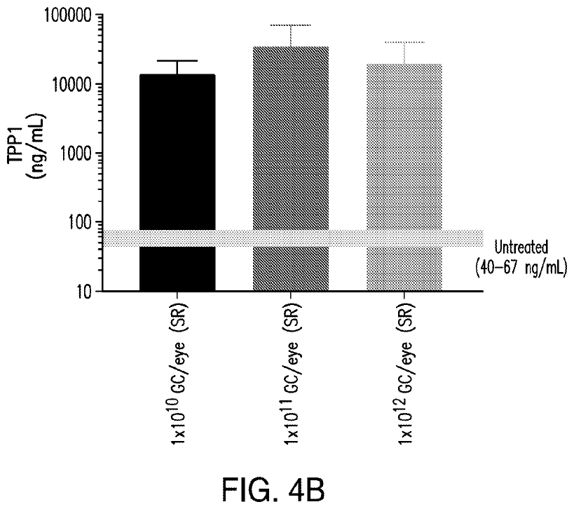

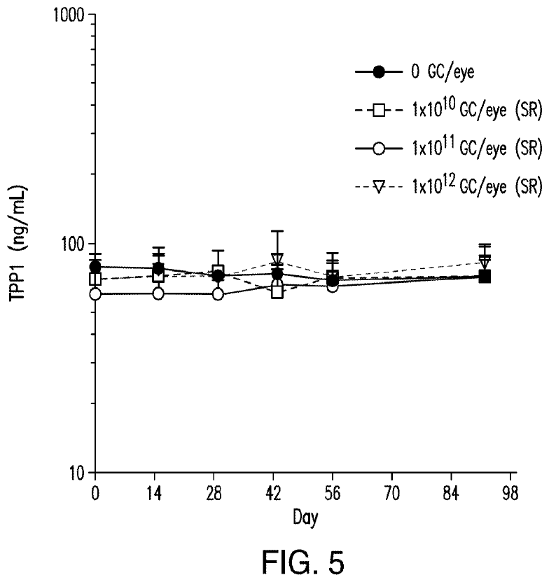

Subretinal injection of Construct I into the eyes of healthy cynomolgus monkeys led to increases in TPP1 concentrations in the aqueous humor over the course of the study as well as in the vitreous humor (terminal sample only) after 3 months (FIGS. 4A and 4B). This was not reflected in the serum, which remained comparable to control animals throughout the study (FIG. 5). Terminal samples from Construct I-treated animals showed an increased incidence of anti-TPP1 antibodies that in some animals may have led to decreases in aqueous TPP1 concentration.

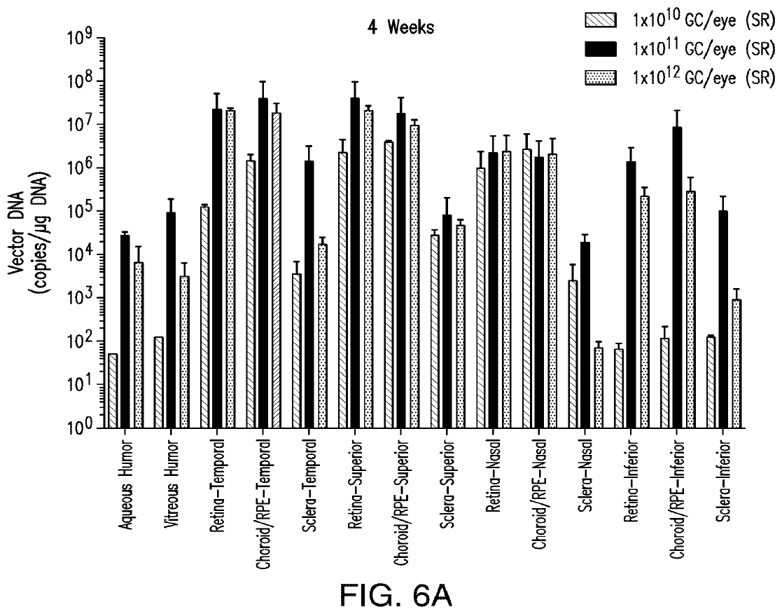

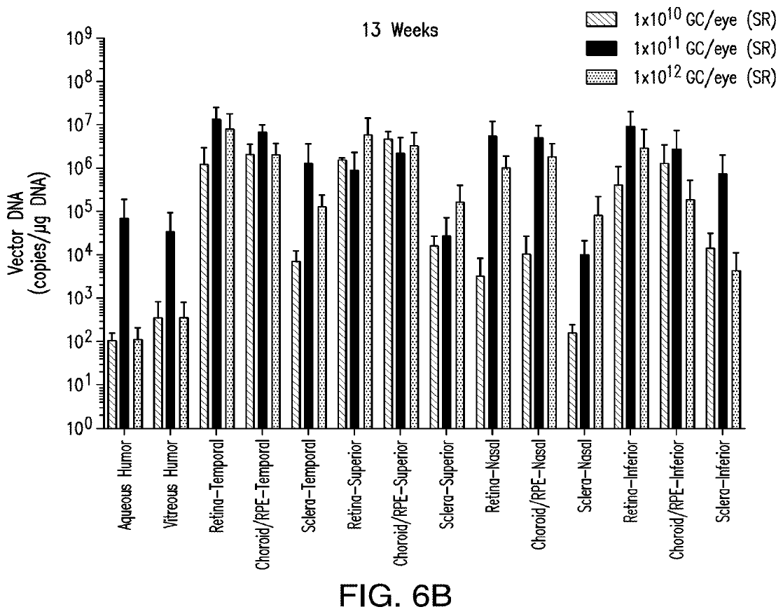

High levels of vector DNA were detected in retina and choroid/RPE at the area of the bleb (temporal) as well as the superior, inferior and nasal quadrants (FIGS. 6A and 6B).

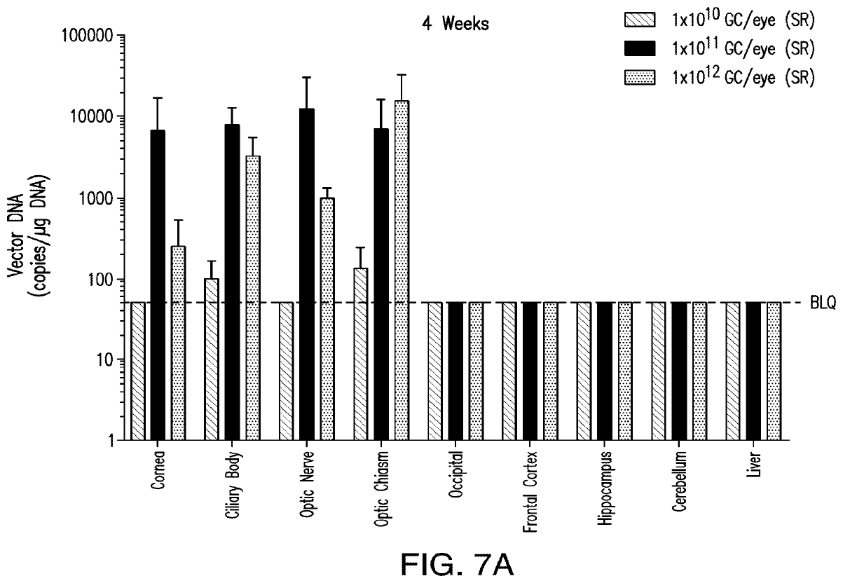

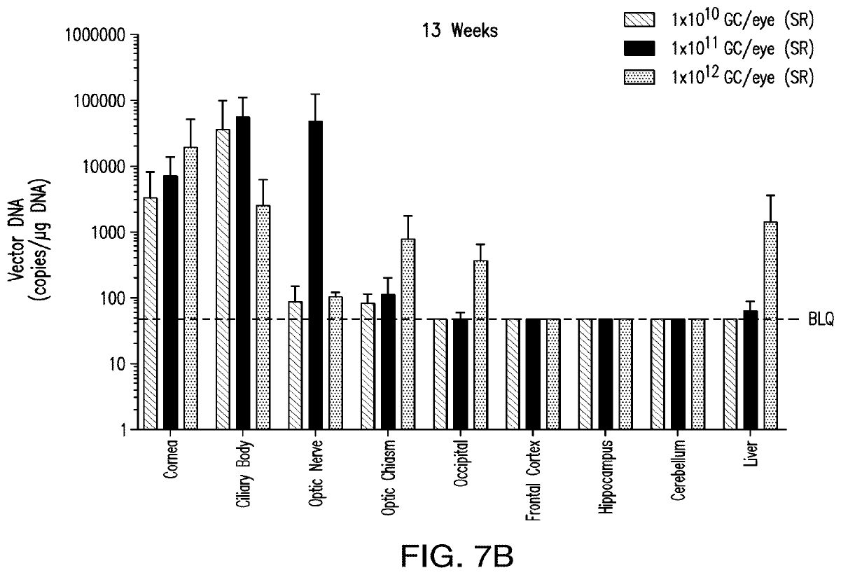

Vector DNA was detected at low levels in the optic nerve, optic chiasm and occipital lobe at ≥1×10¹¹ GC/eye, suggesting limited anterograde tropism along the visual pathway (FIGS. 7A and 7B).

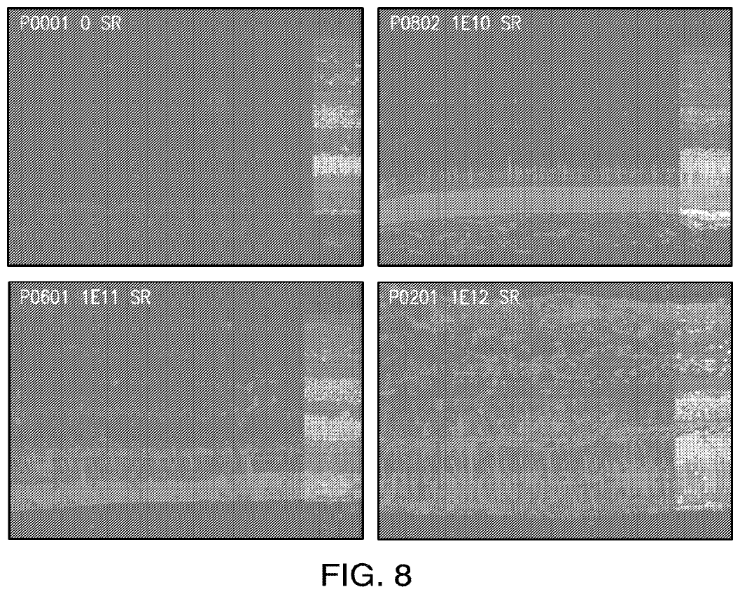

TPP1 immunoreactivity was observed within widespread areas of the retina. At >1×10¹⁰ GC/eye, intense TPP1 immunoreactivity was present in the retinal pigment epithelium, a proportion of the photoreceptor outer segments and only occasional labelling of horizontal or amacrine cells (more evident at 1×10¹¹ GC/eye). In marked contrast at 1×10¹² GC/eye, intense TPP1 immunoreactivity was observed across the entire depth of the retina (FIG. 8). At 1×10¹² GC/eye, TPP1 immunostaining appeared to label all types of neurons present, including retinal ganglion cells and a much higher proportion of photoreceptor cells than at either of the other two doses. Regardless of dose, TPP1 immunoreactivity within the retina appeared to be confined to neurons with no obvious staining of muller glial cells, even adjacent to where the subretinal vector deposits had been placed.

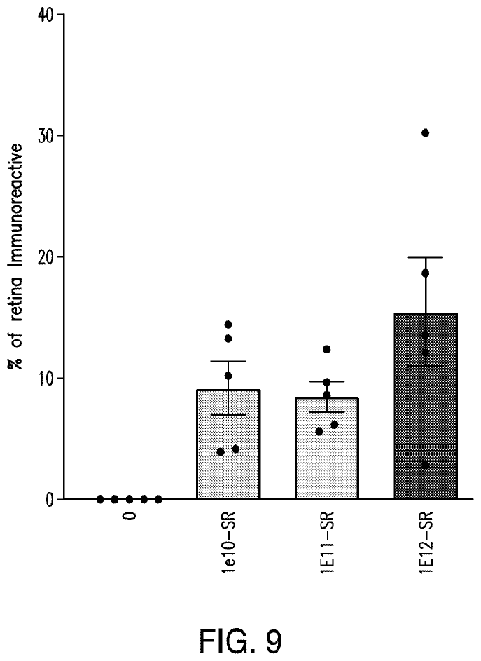

The % area of TPP1 stained retina was greater in Construct I-treated animals than that present in vehicle treated animals (FIG. 9).

6.3.3 Conclusions:

A dose of 1×10¹⁰ GC/eye led to TPP1 concentrations that were approximately 170-fold (aqueous) and 200-fold (vitreous) greater than concentrations of TPP1 in untreated cynomolgus monkeys.

Immunostaining showed intense TPP1 immunoreactivity within the retinal pigment epithelium and in a proportion of the outer segment of photoreceptors, with only occasional labelling of presumed to be horizontal or amacrine cells at 1×10¹⁰ GC/eye.

Data generated in cynomolgus monkeys demonstrates that subretinal injection of Construct I may provide sufficient levels of TPP1 to attenuate the retinal degeneration seen in CLN2 patients.

### 6.4 Example 4: Subretinal Injection of Construct I to Cynomolgus Monkeys Leads to a Favorable Safety Level

The aim of this study was to assess the safety of Construct I, a recombinant adeno-associated virus of serotype 9 (AAV9) containing a human CLN2 (hCLN2) expression cassette, by a 3-month toxicity study in cynomolgus monkeys. In addition to standard endpoints for toxicity studies, this study included assessment of physiological changes in eyes. The juvenile toxicity, reproductive toxicity, and genotoxicity were also discussed in view of high unmet clinical needs in children with CLN2 disease.

6.4.1 Methods

Groups of cynomolgus monkeys were administered Construct I by subretinal injection at doses ranging from 1×10¹⁰ to 1×10¹³ GC/eye. The tripeptidyl peptidase 1 (TPP1) concentration in the serum, aqueous and vitreous humor, and biodistribution were assessed to confirm the transgene expression. The eyes in study were examined by direct and indirect ophthalmoscopy, OCT, electroretinography, fundus photography, and histopathology. The effects were assessed for up to 3 months. Young cynomolgus monkeys aged 2 to 3 years were included for study.

6.4.2 Results

Subretinal injection of Construct I to cynomolgus monkeys demonstrated an elevated and sustained levels of TPP1 biodistribution, indicating a normal transgene expression in this study.

The results suggested that a dose of 1×10¹⁰ GC/eye was the no-observed-adverse-effect level (NOAEL) for this study. The adverse effect or findings included ocular inflammation, OCT and fundus photography findings, and reduced visual function.

Subretinal administration of 1×10¹⁰, 1×10¹¹, 1×10¹², or 1×10¹³ GC/eye of Construct I to male and/or female cynomolgus monkeys was associated with adverse findings at doses ≥1×10¹¹ GC/eye. At doses of 1×10¹¹ or 1×10¹² GC/eye, the ocular inflammation observed in aqueous cell, vitreous haze, or vitreous cell was of a lower severity than that observed at the highest dose. Also, the ocular inflammation observed at lower doses (e.g. 1×10¹¹ or 1×10¹² GC/eye) responded well to systemic and/or topical anti-inflammatory therapy.

An occasional vitreous cell inflammation observed at 1×10¹⁰ GC/eye was not considered as an adverse effect. At 1×10¹⁰ GC/eye, an occasional vitreous cell inflammation decreased in severity by Day 22 but did persist in some eyes through 3 months. One animal showed a more severe inflammation with severe aqueous cell, moderately severe flare, and moderately severe vitreous haze. However, that was observed in one eye only on Day 15, and a topical anti-inflammatory therapy was applied. No vector-related effects on Ocular Color Fundus Photography, ERG, or visual evoked potential were observed at the 1×10¹⁰ GC/eye dose, thereby demonstrating that the vitreous cell inflammation did not correlate with effects on visual function, even though minimal mononuclear cell infiltrates were observed in one eye at the end of the study on microscopic evaluation. This suggested that the occasional vitreous cell inflammation at 1×10¹⁰ GC/eye may not affect visual function, and may be controlled within a mild level.

No neurobehavioral, respiratory, or microscopic (cardiovascular) changes were observed in cynomolgus monkeys throughout the 3-month toxicity study.

No findings suggested an effect on the reproductive system at 1×10¹⁰ GC/eye dose. Vector DNA was not detected in the testes or ovaries of all animals up to the dose of 1×10¹⁰ GC/eye. In contrast, vector DNA was primarily detected in ocular tissues on Days 29 and 92 under the same dose. This distinction supports that 1×10¹⁰ GC/eye may be safe for reproductive system due to a non-detectable vector DNA level therein.

The presence of vector DNA at very low copy numbers at doses ≥1×10¹¹ GC/eye did not suggest an effect on the reproductive system. In one male administered 1×10¹¹ GC/eye subretinally and one male administered 1.0×10¹² GC/eye subretinally, vector DNA was detected in testes at very low levels, which was 340 and 109 copies/μg DNA, respectively, on Day 92. In one female administered into the suprachoroidal space (SCS) at 1×10¹² GC/eye, 344 copies/μg DNA was detected in ovaries on Day 29. No microscopic changes in the testes and ovaries were observed. The absence of such changes in the reproductive system suggests that the very low DNA copies may not be considered an adverse effect, even at doses ≥1×10¹¹ GC/eye up to the highest dose in the study.

Construct I may have a low risk for genotoxicity. The available scientific literature and clinical data have reported that the integration of AAV vectors has not been observed (Wang et al., Adeno-associated virus vector as a platform for gene therapy delivery, Nature Reviews Drug Discovery, 2019, 18: 358-378). Without the integration machinery of wild type AAV, Construct I is unable to replicate in transduced cells. The recently approved AAV9, onasemnogene abeparvovec-xioi (ZOLGENSMA USPI, 2019), supports that recombinant adeno associated viruses (rAAVs) have a low risk for insertional mutagenicity.

The immunotoxicity studies with Construct I may be assessed by immune response. In the study, humoral responses were monitored by detection of antibodies against the AAV9 capsid, and antibodies directed against the transgene product, i.e. anti-TPP1 antibodies in cynomolgus monkeys.

The juvenile toxicity of Construct I may be assessed in the cynomolgus monkeys aged 2 to 3 years. According to the European Medicines Agency guideline, this age range likely covers prepubertal monkeys and therefore includes age of gestation in the evaluation (EMEA/CHMP/SWP/169215/2005).

6.4.3 Conclusions

A dose level of 1×10¹⁰ GC/eye was a NOAEL for subretinal administration of Construct I to cynomolgus monkeys in this example. In clinical study, a starting dose level of 2×10¹⁰ GC/eye may be conducted as the NOAEL Ocular inflammation observed at doses of 1×10¹¹ or 1×10¹² GC/eye responded well to systemic and/or topical anti-inflammatory therapy.

Data generated from the 3-month toxicity study in cynomolgus monkeys demonstrates that subretinal administration of Construct I may provide a favorable safety profile. No neurobehavioral, respiratory, or microscopic changes were described following either SR or SCS administration of Construct I. No specific juvenile toxicity, reproductive toxicity, or genotoxicity were observed.

### 6.5 Example 5: Evaluation of Construct I in a Retina-On-a-Chip Model of CLN2 Disease

The aim of this study was to develop a physiologically relevant three-dimensional in vitro model of the human retina in CLN2 disease by combining human induced pluripotent stem cells lines (hiPSC) retinal organoids (ROs) with hiPSC RPE in a Retina-on-a-Chip (RoC) model. This novel microphysiological platform enables enhanced inner and outer segment formation and preservation, a direct interplay between RPE and photoreceptors, as well as a precisely controllable vasculature-like perfusion. As well as characterising the disease phenotype, which is not possible in animal models or understood in CLN2 children, this model provides support for the mechanism of action and potential benefit for Construct I.

6.5.1 Method

Skin fibroblasts collected from children prior to death from CLN2 disease were reprogrammed into human induced pluripotent stem cells lines and differentiated into RPEs and retinal organoids (ROs) as described in Achberger et al. eLife 2019, 8, e46188, which is incorporated herein by reference.

6.5.2 Results

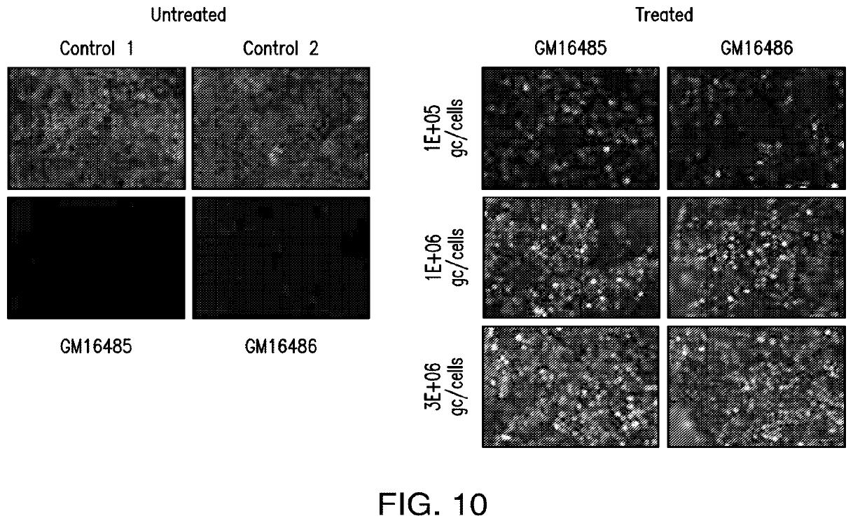

Unlike the control RPEs, CLN2 RPEs do not express TPP1. Restoration of TPP1 expression was shown in CLN2 RPEs 21 days post-transduction with Construct I (FIG. 10).

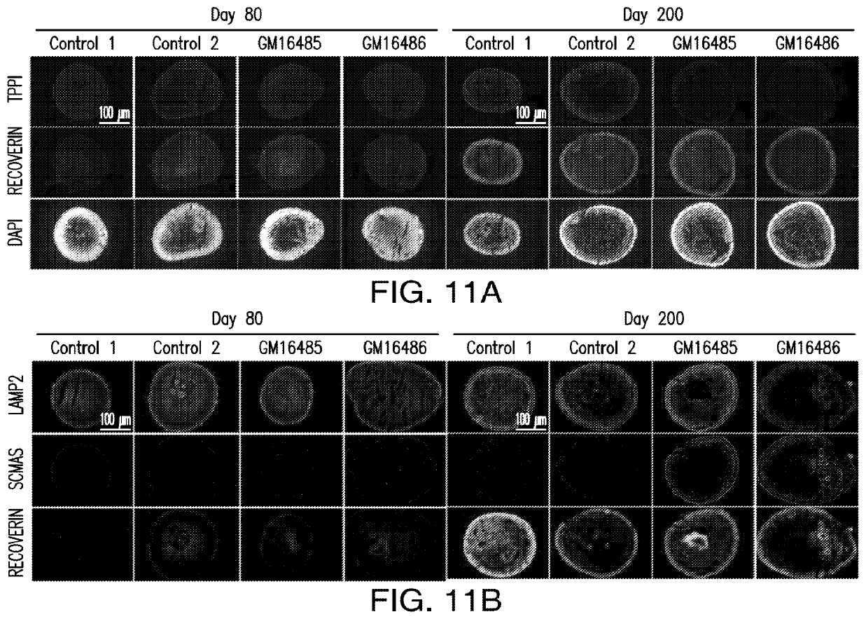

After isolation of RPEs, ROs were further maturated. Recoverin, a photoreceptor marker, was positive at Day 200 of differentiation, and absent at Day 80. In control lines, TPP1 expression was only detected in ROs at Day 200, mostly in the region of photoreceptors, suggesting spatial and temporal specificity of TPP1 expression. Interestingly, ATP Synthase subunit C (SCMAS), one of the accumulated substrates identified in CLN2, was present in CLN2 ROs only at 200 days in the region of photoreceptors where TPP1 expression is detected in control ROs (FIG. 11).

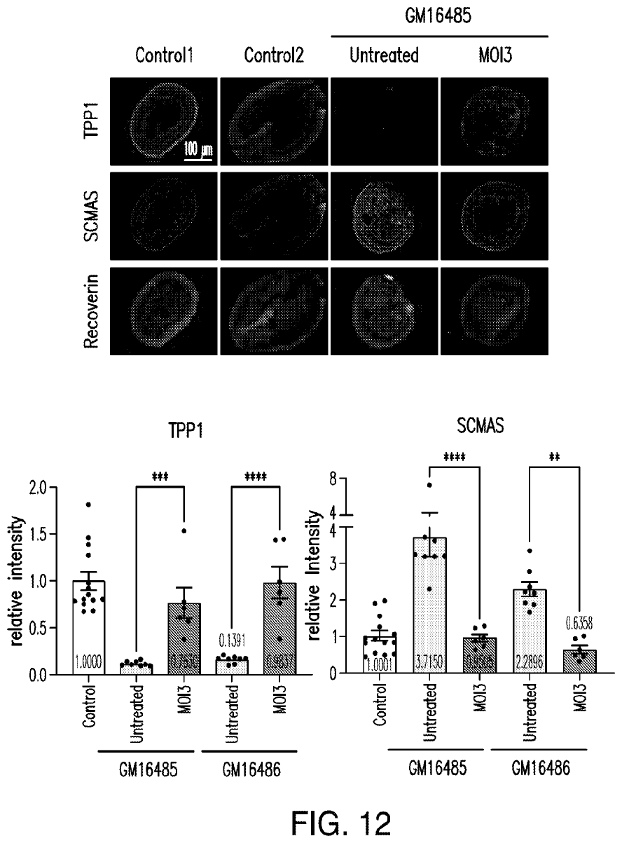

Treatment of CLN2 ROs with Construct I at Day 88 prior to detection of SCMAS accumulation showed TPP1 expression alongside prevention SCMAS accumulation, in contrast to untreated CLN2 ROs, suggesting that early intervention can slow down or prevent storage material accumulations in ROs (FIG. 12).

6.5.3 Conclusions

The data generated in human CLN2 RPEs with Construct I show that human CLN2 RPEs and ROs lack TPP1 and treatment with Construct I can restore expression of TPP1 after treatment. Furthermore, if CLN2 ROs are left untreated, there is accumulation of SCMAS, a hallmark of CLN2 disease, that was prevented by pre-treatment with Construct I. This data shows that transduction of ROs (which includes photoreceptors) with Construct I prevents the accumulation of lysosomal storage material associated with CLN2 degeneration.

### 6.6 Example 6: Protocol for Evaluation of Construct I in a Retina-On-a-Chip Model of CLN2 Disease

The aim of this study is to assess the efficacy of Construct I, a recombinant adeno-associated virus of serotype 9 (AAV9) containing a human CLN2 (hCLN2) expression cassette, in treating ocular manifestations of CLN2 in a human subject using a Retina-on-a-Chip model.

6.6.1 Differentiation of Patient and Control iPSC to Retinal Organoids (RO)

Four human induced pluripotent stem cells lines (iPSCs, 2 CLN2 patients) and 2 control lines will be thawed and differentiated to retinal organoids (RO) following the retinal differentiation protocol described in Zhong et al. Nat. Commun. 2013, 5, 4047 and Achberger et al. eLife 2019, 8, e46188, each of which is incorporated herein by reference. RO will be cultured for a minimum of 3-6 months. From differentiated RO, pigmented RPE cell clumps will be harvested by manual dissection. These RPE cells will be dissociated and cultured adherently. RPE cells from all available lines will be cultured or frozen until time of use.

6.6.2 Treatment of Retinal Organoids with Construct I

RO at day 80 of the available lines produced in Section 6.6.1 will be selected according to internal quality criteria (e.g. formation of segments, epithelial striation, absence of large necrotic areas).

Selected organoids will be put into e.g. a 96 well format and subjected to Construct I. For this, 3 different concentrations will be applied to the available patient lines. Moreover, one concentration of an empty AAV will be applied to one patient line to evaluate the specificity of the TPP1 gene expression effect. Effects of the therapy on the cells will be monitored 5 weeks after initial addition of the Construct I. Endpoint methods will include RNA analysis (4 biological replicates per condition) via qPCR and protein analysis via immunocytochemistry (10 Samples per condition). RO will be stained for different retina cell type markers, lysosomal markers (e.g. LAMP1 or LAMP2), TPP1 expression and mitochondrial ATP synthase subunit C (SCMAS).

The foregoing procedure will be repeated with RO at day 120.

6.6.3 Treatment of the Retina-On-a-Chip with Construct I

For Retina-on-a-Chip (RoC) experiments, RO at day 80 and day 120 from all available lines will be chosen by the same criteria applied to Section 6.6.2. RPE of the available lines produced in Section 6.6.1 will be selected according to internal quality criteria (passage, culture time, pigmentation, hexagonal cell shape and general appearance). Three different concentrations of Construct I as well as vehicle control will be applied to the available patient lines. Effects of the therapy on RoCs will be monitored 5 weeks after initial addition of Construct I. Endpoint methods will include RNA analysis via qPCR and protein analysis via cryosectioning followed by immunocytochemistry (8 samples per condition). RoCs will be stained for different retina cell type markers, lysosomal markers (e.g. LAMP1 or LAMP2), TPP1 expression and SCMAS.

### 6.7 Example 7: Protocol for Treatment of Human RPE Organoids with Construct I and Investigation of SCMAS Accumulation

The aim of this study is to treat human RPE organoids with Construct I and investigate SCMAS accumulation through flow cytometry, single cell RNA sequencing and high resolutions confocal microscopy in RPE organoids and retinal organoids

6.7.1 Differentiation of Patient and Control iPSC to Retinal Organoids (RO)

Four human iPSC lines, 2 patients and 2 control lines will be thawed and differentiated to retinal organoids (RO) as in Section 6.6.1. From differentiated RO, pigmented RPE cell clumps will be harvested by manual dissection. These RPE cells will be cultured in suspension to form RPE organoids.

6.7.2 Treatment of Retinal Pigmented Epithelial Cell Organoids (RPEorg) with TPP1 Gene Therapy

According to our data, adherent RPE cell, that are cultured on a monolayer for a limited time period, do not represent an accurate model to recapitulate disease phenotypes such as ATP-C deposits. On the other hand, RPEorg, due to their 3D structure and the long-term cultivation, showed to be a solid model to study TPP1 mutation effects and ATP-C accumulation, thus representing an interesting platform to evaluate TPP1 Gene Therapy effect in RPE cells. For this purpose, RPEorg at day >150 (onset of the phenotype) of the available lines produced in Section 6.7.1 will be selected according to internal quality criteria (e.g. uniform pigmentation, absence of non-RPE areas). Selected RPEorg will be put into e.g. a 96 well format and subjected to treatment with Construct I. Effects of the therapy on the cells will be monitored 5 weeks after initial addition of the Construct I. Endpoint methods will include RNA analysis (4 biological replicates per condition) via qPCR and protein analysis via immunocytochemistry (10 Samples per condition). RO will be stained for TPP1 expression, SCMAS and lysosomal markers (e.g. LAMP1 or LAMP2).

6.7.3 Analysis of TPP1 Mutation and TPP1 Gene Therapy Effect on RPEorg

RPEorg from 2 control lines, 2 patient lines and 2 patient lines treated with one concentration of TPP1 Gene Therapy (e.g. Construct I) at day >150 will be selected 5 weeks after AAV treatment, dissociated and will undergo single cell RNA sequencing (sc-RNAseq). The sc-RNAseq evaluation will determine the effect of TPP1 mutation on gene expression, including but not limited to SCMAS accumulation. Finally, the analysis will identify consequences of TPP1 Gene Therapy (e.g. Construct I) on RPE cell signaling, survival and apoptosis.

6.7.4 Cell Type Specific Analysis of TPP1 Mutation and TPP1 Gene Therapy effect on ROs

ROs from 2 control lines, 2 patient lines and 2 patient lines treated with one concentration of TPP1 Gene Therapy (e.g. Construct I) at day 120 will be selected 5 weeks after AAV treatment, dissociated and will undergo single cell RNA sequencing (sc-RNAseq). The analysis will evaluate the retinal cell types expressing TPP1 (in the control lines) and the cell types more efficiently infected by the TPP1 Gene Therapy (in the patient lines). Moreover, the sc-RNAseq evaluation will determine the effect of TPP1 mutation on gene expression, including but not limited to SCMAS accumulation. Finally, the analysis will identify consequences of TPP1 Gene Therapy on cell signaling, survival and apoptosis.

6.7.5 Investigation of SCMAS Accumulation

In order to identify the retinal cell types affected by SCMAS deposits and highlight a correlation between TPP1 expression/absence and SCMAS, ROs will be analyzed by flow cytometry (FC) after staining with SCMAS, TPP1 and one specific antibody for each retina cell type (Rods, Cones, Müller Glia, Horizontal Cells, Amacrine Cells, Bipolar Cells).

For this purpose, protocol for FC detection of these antibodies will first be established. Subsequently, 3-5 ROs from 2 control lines, 2 patient lines and 2 patient lines treated with one concentration of TPP1 Gene Therapy (e.g. Construct I) at day 120 will be selected 5 weeks after AAV treatment, dissociated and will undergo flow cytometry analysis after staining with ATP-C, TPP1 and one specific antibody for each retina cell type.

Finally, the retinal cell types that show SCMAS accumulation will be further analyzed by high resolution confocal microscopy. For this purpose, 3-5 ROs from 2 control and 2 patient lines at >155 days with be stained for SCMAS and individual markers or retinal subtypes for evaluation of SCMAS deposit morphology and cellular localization.

### 6.8 Example 8: An Expanded CLN2 Clinical Rating Scale Motor (CLN2 CRS-MX) Improves the Evaluation on Ambulatory Function

In late infantile neuronal ceroid lipofuscinosis 2 (CLN2) disease, the rapid progression of the disease was originally quantified using the Hamburg CLN2 scale. The Hamburg motor domain was subsequently adapted into the CLN2 Clinical Rating Scale Motor (CLN2 CRS-M) and used to quantify loss of motor function in the natural disease course compared to the treatment response on cerliponase alfa intraventricular enzyme replacement therapy (ERT). The CLN2 disease impairments includes ataxia, hypotonicity, hypertonicity, and weakness. In children, however, the evolving phenotype on ERT is less perceptible because the disease progression for children is slower.

The aim of this study was to provide a broader and more granular measurement, termed CLN2-CRS-MX, to assess impacts of CLN2 disease impairments on ambulatory capacity.

6.8.1 Methods

The derivation of items was based on an identification of key CLN2 disease concepts of interest.

The key concepts were identified from a targeted literature review, clinician expert interviews, two virtual caregiver focus groups and ongoing biweekly meetings for one year with 6 CLN2 clinician experts.

The clinician interviews and caregiver focus groups discussed the key symptoms and impacts of CLN2 specifically related to motor function, differences in disease progression in ERT-treated and ERT-naïve patients, and how to improve the granularity of motor function assessment in the CLN2 CRS to make it more useful for assessing treatment benefit in a clinical trial.

The iterative developmental process included pilot application and numerous item revisions. Tracking matrices were used to document all scale iterations and the rationale for changes.

The inter-rater reliability, i.e. the level of agreement, between the clinicians was calculated as percent agreement across 4 raters.

Assessments were administered and scored by a primary clinician and also independently scored by an observer clinician.

Each assessment was videotaped and scored independently by two additional clinicians.

Thirty patients aged 20 months to 16 years (mean 7.8 years) were each scored by 4 clinicians on the CLN2 CRS-MX scale. They were all currently receiving ERT.

6.8.2 Results

The study developed a detailed administrative, scoring, and training manual of the CLN2 CRS-MX scale.

The study developed a typical ambulation task for determining symptom progression. Generally, clinicians and caregivers characterized CLN2 disease by a marked progression in symptoms that deleteriously impact ambulation. Truncal hypotonia, extremity hypertonicity and ataxia were commonly reported symptoms that impacted motor function. As symptoms worsen, an increase in the need for assistance, for example, holding a hand, hands or trunk for balance, was reported, until patients were no longer able to walk. In this study, clinicians supported expansion of the 10-step ambulatory task on the original CLN2 CRS-M, to an ambulatory task that extended the distance walked, considered ability to safely stop, graded a change in direction, and measured the level of assistance that was necessary to ambulate.

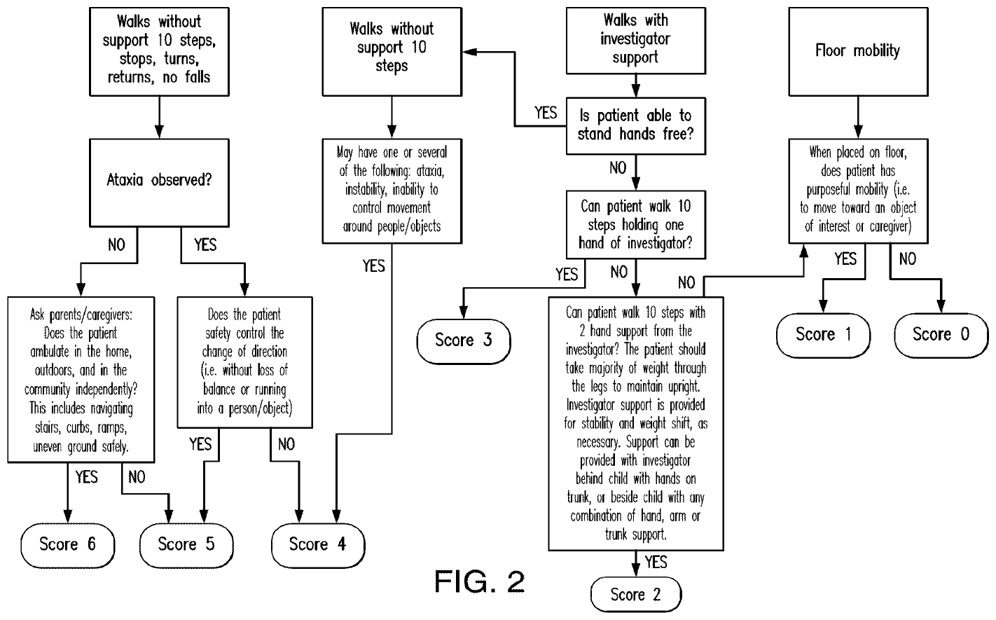

Table 2 provides an outline of the rating criteria and comparisons to the original CLN2 CRS-M. FIG. 2 outlines the CLN2 CRS-MX scoring flow chart to determine the mobility rating.

TABLE 2
CLN2 CRS-M and CLN2 CRS-MX Rating Criteria.

| CLN2 | | CLN2 | |
|---|---|---|---|
| CRS-M | CLN2 CRS-M | CRS-MX | |
| Level | Rating Criteria | Level | CLN2 CRS-MX Rating Criteria |
| A | Grossly normal | 6 | Normal gait, no ataxia, no pathologic falls. Ambulates in |
| | gait. No | | the home, outdoors and in community independently. |
| | prominent | | Child is able to navigate uneven ground safely, without |
| | ataxia, no | | falling (stairs, curbs, ramps etc.) |
| | pathologic falls | | |
| 2 | Independent | 5 | Independent gait as defined by ability to walk |
| | gait, as defined | | forward without support 10 steps, stops and turn |
| | by ability to | | 180° and return. Ataxia may be present but can |
| | walk without | | safely change direction without loss of balance or |
| | support for 10 | | running into a person or object |
| | steps. Will have | 4 | Independent gait defined as ability to walk |
| | obvious | | forward without support for 10 steps. May have |
| | instability and | | obvious instability or ataxia and decreased ability |
| | may have | | to control movement around people or objects |
| | intermittent falls | | |
| 1 | No unaided gait. | 3 | Walks forward 10 steps with one-hand support |
| | Requires | 2 | Walks forward 10 steps with two-hand support. The |
| | external | | patient should take the majority of weight through the legs |
| | assistance to | | to maintain upright. Support is provided for stability and |
| | walk or can | | weight shift as necessary. Support can be provided behind |
| | crawl only | | the child with hands on the trunk or beside the child with |
| | | | any combination of hand, arm or trunk support |
| | | 1 | Floor Mobility: when placed on floor, patient has |
| | | | purposeful mobility to move to an object of interest. |
| | | | Mobility can include crawling, scooting or rolling |
| 0 | Immobile, can | 0 | Can no longer walk, scoot, roll or crawl |
| | no longer walk | | |
| | or crawl | | |

In the inter-rater agreement, the level of agreement between the clinicians for the CLN2 CRS-MX was 1.0, indicating complete/perfect agreement (see Landis, JR, Koch, GG. An application of hierarchical kappa-type statistics in the assessment of majority agreement among multiple observers, Biometrics, 1977. Level of agreement range 0.81-0.99).

6.8.3 Conclusions

The CLN2 CRS-MX provided more granularity, improved item relevance, and increased the number of response options in the measurement of CLN2 disease's impact on ambulatory capacity.

The scale was designed for children with CLN2, but may have relevance for other neuromuscular or neurodegenerative disorders that present with similar disease impairments.

### 6.9 Example 9: An Expanded CLN2 Clinical Rating Scale Language (CLN2 CRS-LX) Improves the Evaluation on Language Function

The loss of language function in the natural CLN2 disease course compared to the treatment response on ERT was quantified using the CLN2 Clinical Rating Scale Language (CLN2 CRS-L). However, children's disease progression and phenotype evolution in ERT treatment is slower and less perceptible. The aim of this study was to provide a broader and more granular measurement, termed CLN2 CRS-LX, to capture changes in expressive language and non-verbal communication competencies.

6.9.1 Methods

The derivation of items was based on an identification of key CLN2 disease concepts of interest.

The key CLN2 disease concepts were identified from a targeted literature review [not specified in the materials], clinician expert interviews, two virtual caregiver focus groups and ongoing biweekly meetings for one year with 6 CLN2 clinician experts.

The clinician interviews and caregiver focus groups discussed the key symptoms and impacts of CLN2 specifically related to language function, differences in disease progression in ERT-treated and ERT-naïve patients and how to improve the granularity of language function assessment in the CLN2 CRS to make it more useful for assessing treatment benefit in a clinical trial.

The iterative developmental process included pilot application and numerous item revisions. Tracking matrices were used to document all scale iterations and the rationale for changes.

The inter-rater reliability, i.e. the level of agreement, between the clinicians was calculated as percent agreement across 4 raters.

Assessments were administered and scored by a primary clinician and also independently scored by an observer clinician.

Each assessment was videotaped and scored independently by two additional clinicians.

Thirty patients aged 20 months to 16 years (mean 7.8 years) were each scored by 4 clinicians on the CLN2 CRS-MX. They were all currently receiving ERT.

6.9.2 Results

The study developed a detailed administrative, scoring and training manual of the CLN2 CRS-LX. The manual contains specific guidance on use of prompts to determine expressive language and non-verbal communication competencies.

The CLN2 CRS-LX measured a child's expressive language ability to communicate wants and needs, including babbling, vocabulary and phrase development, and non-intelligible vocalizations and gestures. Generally, clinicians and caregivers reported that children with CLN2 experience progressive language loss that impacted daily activities. They reported a peak word count of 20-100 words at an age range from 2 to 3.5 years and that children with CLN2 used a range of communication strategies including single and double-word phrases, non-intelligible vocalizations and gestures. In this study, clinicians supported the development of an expanded CLN2 CRS for language that differentiated functional expectations by age, included more response options, and considered vocabulary and phrase development, vocalizations and gestures.

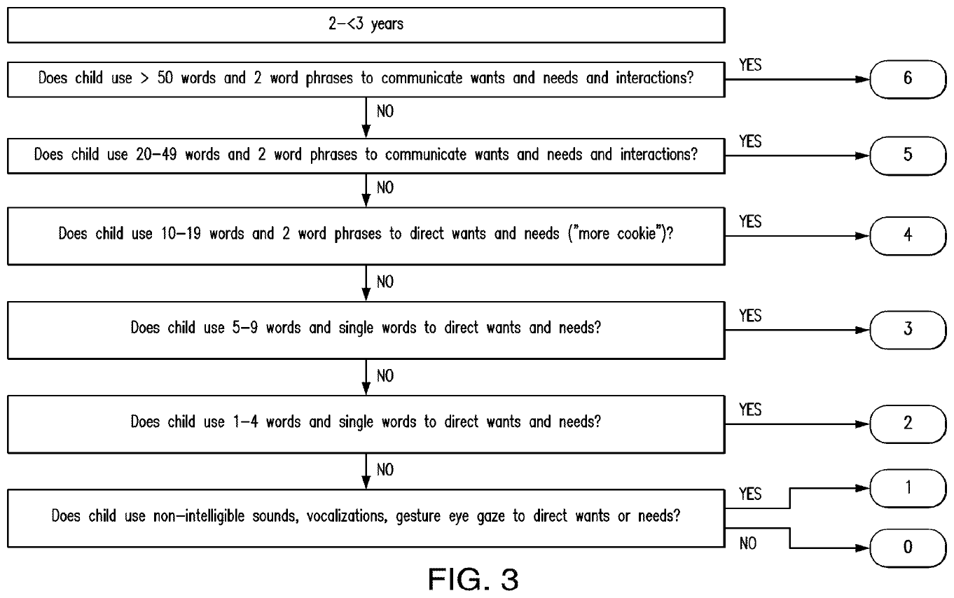

An outline of the rating criteria and comparisons to the original CLN2 CRS-L are provided in Tables 3 and 4. Scoring flowcharts were developed for each CLN2 CRS-LX age category (1 to <2 years, 2 to ≤3 years and >3 years). FIG. 3 shows the 2 to <3 Year flowchart as an example.

TABLE 3
CLN2 CRS-L Rating Criteria.

| CLN2 | |
|---|---|
| CRS-L | |
| Level | CLN2 CRS-L Rating Criteria |
| 3 | Apparently normal language. Intelligible and grossly age- |
| | appropriate. No decline noted yet |
| 2 | Language has become recognizably abnormal: some intelligible |
| | words may form short sentences to convey concepts, requests or |
| | needs. This score signifies a decline from a previous level of |
| | ability (from the individual maximum reached by the child) |
| 1 | Hardly understandable. Few intelligible words |
| 0 | No intelligible words or vocalizations |

TABLE 4
CLN2 CRS-LX Rating Criteria

| CLN2 | | | |
|---|---|---|---|
| CRS- | | | |
| MX | | CLN2 CRS-LX Rating | CLN2 CRS-LX |
| Level | CLN2 CRS-LX Rating Criteria | Criteria | Rating Criteria |
| | 1 to <2 years | 2 to <3 years | ≥3 years |
| 6 | Language is normal based on age criteria for | Child uses ≥50 words | Child uses 100+ words |
| | words, consonant vowel combinations and | and 2 word phrases to | and 3-4 word phrases to |
| | gestures | communicate wants, | communicate wants, |
| | 1-2 words at 12 m | needs and interactions | needs and interactions |
| | 8 words by 15 m | | |
| | 15 words by 18 m | | |
| | Uses consonant vowel | | |
| | combinations (ex. baba, mimi) | | |
| | Points or gestures (waves bye bye, | | |
| | shakes head for no, pushes away | | |
| | object, reaches to be picked up | | |
| 5 | Child says less words than criteria | Child uses 20-49 words | Child uses 50-99 words |
| | for age. | and at least 2 word | and at least 3 word |
| | Child uses at least 2 consonant | phrases to communicate | phrases to communicate |
| | vowel combinations and gestures | wants, needs and | wants, needs and |
| | | interactions | interactions |
| 4 | Child uses a consonant vowel combination | Child uses 10-19 words | Child uses 20-49 words |
| | and point, gesture or back and forth eye gaze | and at least 2 word | and at least 2 word |
| | | phrases to direct wants | phrases to direct wants, |
| | | and needs | needs and interactions |
| 3 | Child babbles (vowel sounds) and uses | Child uses 5-9 words | Child uses 5-19 words |
| | gesture or back and forth eye gaze | and single words to | and single words to |
| | | direct wants and needs | direct wants and needs |
| 2 | Child vocalizes mood such as pleasure, | Child uses 1-4 words | Child uses 1-4 words |
| | displeasure, eagerness or satisfaction in | and at least single words | and at least single words |
| | response to any of the following; social | to direct wants and needs | to direct wants and needs |
| | interaction, initiation or interruption of play | | |
| 1 | Child uses cry or non-intelligible sounds to | Child uses non- | Child uses non- |
| | direct wants and needs | intelligible sounds, | intelligible sounds, |
| | | vocalizations, gestures | vocalizations, gestures |
| | | or eye gaze to direct | or eye gaze to direct |
| | | wants and needs | wants and needs |
| 0 | Child does not use vocalization or gestures | Child does not use | Child does not use |
| | to communicate wants or needs | vocalizations or gestures | vocalizations or gestures |
| | | to communicate wants | to communicate wants |
| | | and needs | and needs |

In the inter-rater agreement, the level of agreement between the clinicians for the CLN2 CRS-LX was 0.933, indicating almost perfect agreement (see Landis, JR, Koch, GG. An application of hierarchical kappa-type statistics in the assessment of majority agreement among multiple observers, Biometrics, 1977. Level of agreement range 0.81-0.99).

6.9.3 Conclusions

The CLN2 CRS-LX provided more granularity, improved item relevance, and increased the number of response options in the measurement of CLN2 disease's impact on expressive language and non-verbal communication competencies.

The scales was designed specifically for children with CLN2, but may have relevance for other neuromuscular or neurodegenerative disorders that present with similar disease impairments.

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed is:

1. A method of treating ocular manifestations associated with CLN2 Batten disease in a subject in need thereof, said method comprising administering to the eye of said subject a recombinant adeno-associated virus (rAAV) comprising a capsid and a vector genome, wherein the rAAV is AAV9, and wherein the vector genome comprises:
a. an AAV 5′ inverted terminal repeat (ITR);
b. a promoter;
c. a CLN2 coding sequence encoding a human tripeptidyl peptidase 1 (TPP1) protein; and
d. an AAV 3′ ITR.

2. The method of claim 1, wherein the AAV 5′ ITR and/or AAV3′ ITR is from AAV2.

3. The method of claim 1 or 2, wherein the coding sequence of (c) is a codon optimized human CLN2, which is at least 70% identical to the native human coding sequence of SEQ ID NO: 2.

4. The method of claim 1 or 2, wherein the coding sequence of (c) is SEQ ID NO: 3.

5. The method of any one of claims 1-4, wherein the promoter is a chicken beta actin promoter.

6. The method of any one of claims 1-4, wherein the promoter is a hybrid promoter comprising a CBA promoter sequence and cytomegalovirus enhancer elements.

7. The method of any one of claims 1-6, wherein the vector genome further comprises a polyA.

8. The method of claim 7, wherein the polyA is a synthetic polyA or from bovine growth hormone (bGH), human growth hormone (hGH), SV40, rabbit β-globin (RGB), or modified RGB (mRGB).

9. The method of any one of claims 1-8, further comprising an intron.

10. The method of claim 9, wherein the intron is from CBA, human beta globin, IVS2, SV40, bGH, alpha-globulin, beta-globulin, collagen, ovalbumin, or p53.

11. The method of any one of claims 1-10, further comprising an enhancer.

12. The method of claim 11, wherein the enhancer is a CMV enhancer, an RSV enhancer, an APB enhancer, ABPS enhancer, an alpha mic/bik enhancer, TTR enhancer, en34, ApoE.

13. The method of any one of claims 1-12, wherein the vector genome is about 3 kilobases to about 5.5 kilobases in size.

14. The method of any one of claims 1-13, wherein the vector genome is about 4 kilobases in size.

15. The method of any one of claims 1-14, wherein 2×10¹⁰ genome copies per eye of the rAAV are administered.

16. The method of any one of claims 1-14, wherein 6×10¹⁰ genome copies per eye of the rAAV are administered.

17. The method of any one of claims 1-16, wherein the subject has a change from baseline in central retinal thickness (CRT) as measured by SD-OCT.

18. The method of any one of claims 1-16, wherein the subject has a change from baseline in outer nuclear layer (ONL) thickness as measured by SD-OCT.

19. The method of any one of claims 1-16, wherein the subject has a change from baseline in pupillary light reflex as measured by pupillometry.

20. The method of any one of claims 1-16, wherein the subject has a change from baseline in macular thickness/volume as measured by SD-OCT.

21. The method of any one of claims 1-16, wherein the subject has a change from baseline in full retinal thickness as measured by SD-OCT.

22. The method of any one of claims 1-16, wherein the subject has a change from baseline in the retinal nerve fiber layer as measured by SD-OCT.

23. The method of any one of claims 1-16, wherein the subject has a change from baseline in the inner nuclear layer as measured by SD-OCT.

24. The method of any one of claims 1-16, wherein the subject has a change from baseline in the photoreceptor (PR) plus in the retinal pigment epithelium (RPE) as measured by SD-OCT.

25. The method of any one of claims 1-16, wherein the subject has a change from baseline in the outer segment plus the RPE (OS+RPE) as measured by SD-OCT.

26. The method of any one of claims 1-16, wherein the subject has a change from baseline in the ellipsoid zone (EZ) as measured by SD-OCT.

27. The method of any one of claims 1-16, wherein the subject has a delay in the onset of retinal degradation compared to a comparable clinical progression with standard of care.

28. The method of any one of claims 1-16, wherein the subject has a delay in the onset of visual loss compared to a comparable clinical progression with standard of care.

29. The method of any one of claims 1-28, wherein the method results in detectable TPP1 expression levels in the vitreous humour and/or the aqueous humour of the eye of the subject within 3 months of administration of the rAAV to the subject.

30. The method of claim 29 wherein the levels of TPP1 expression in vitreous humour and/or the aqueous humour were undetectable prior to administration of the rAAV.

31. The method of claim 29 or 30, wherein the levels of TPP1 expression in the serum of the subject remain undetectable.

32. The method of any one of claims 1-31, wherein the subject is concurrently receiving intracerebroventricular Brineura® enzyme replacement therapy.

33. The method of any one of claims 1-32, wherein the subject is human.
